SEQUENCE LISTING (62 sequences)

SEQ ID NO: 1 - protein, 554 aa, synthetic construct
MAKSEGKVNGATLMARALQQQGVQYMFGIVGFPVIPIAIAAQREGITYIGMRNEQSASYAAQAASYLTGRPQACLVVSGPGVVHALAGLANAQVNCWPMLLIGGASAIEQNGMGAFQEERQVLLASPLCKYAHQVERPERIPYYVEQAVRSALFGRPGAAYLDMPDDVILGEVEEAAVRPAATVGEPPRSLAPQENIEAALDALQSAKRPLVIVGKGMAWSRAENEVRQFIERTRLPFLATPMGKGVMPDDHPLSVGGARSHALQEADLVFLLGARFNWILHFGLPPRYSKDVRVIQLDLSAEEIGNNRQAEVALVGDGKAIVGQLNQALSSRQWFYPAETPWREAIAAKIAGNQAAVAPMIADNTSPMNYYRVYRDIAARLPRNAIIVGEGANTMDIGRTQMPNFEPRSRLDAGSYGTMGIGLGFAVAAAAVHPGRPVIAVQGDSAFGFSGMEFETAARYGMPIKVIILNNGGIGMGSPAPRDGQPGMPHALSHDARYERIAEAFGGAGFYVTDSAELGPALDAAMAFKGPAIVNIKIAATADRKPQQFNWHG

SEQ ID NO: 2 - protein, 552 aa, synthetic construct
MAEINGAALIAKCLKQQGVKELFGVVGIPVTGIANAAQKEGIRYIGTRHEQAAGFAAQAVSYLRGHVGVALTVSGPGMTNAITALGNAWANCWPMLLLGGSTDLAFAHRGGFQVAPQMEAARPFCKWVAQPARVEDIPHLIEMGVRTAWYGRPGPVYIDLPADIIEAMVDEASLTYPGPVSPPIRMAAPPELVAEAVQTLRSARKPLLIVGKGAAWSDAATEVRRIVDSTNIPVLPTPMGKGVVPDDHPSIVSAARSYALKNADLIVLAGARLNWILHFGMPPRFNPETRVIQIDLAQEEIGNNLPATVGLTGDLKAILAQMVAQLEETPWKCDDRGAWKAGLAAEVAKKKTELKPALVSDEVPMGYFRPLQEIQKVLPRDAIIVSEGASTMDISRSVLENYQPRNRLDAGSWGTMGGATGFALASAVVHPERRVIALMGDASFGFSGMEVEVAARHRLPITWIVFTNGGIVSGVANLPKDGPLPVNVFQPGARYEKIMEAFGGKGFYCETPDQLARALRTAFDSGETALINVAIAPTAKKAPQTYSHWSSR

SEQ ID NO: 3 - protein, 551 aa, synthetic construct
MGQITGAQIVARALKQQGVEYMFGIVGIPVIPIAMFAQREGIKFYGFRNEQSASYAAAAVGYLTGRPGVCLGVSGPGMIHGVAGMANAWSNCWPMILIGGANDSYQNGQGAFQEAPQIEAARPFAKYCARPDSLARLPFYVEQAVRTSIYGRPGAVYLDLPGDIITGAMEEEDVHFPPRCPDAPRMMAPQESIDAAMAALKSAERPLVIVGKGAAYSRAENEVREFLETTQLPYLASPMGKGVMPDDHPLSIAPARSAALLGADVILLMGARLNWMMHFGHPPRFDPKVRVIQMDISAEEIGTNVPTEVALVGDAKAITTQLNASLKQQPWQYPSETTWWTGLRKKIDENGATVAEMMADESVPMSYYRVYREIRDLIPNDAIIQNEGASTMDIGRTLMPNFLPRHRLDAGSFGTMGVGLGQAIAAAAVHPDKHVFCIEGDSAFGFSGMEVETAARYGMKNITFIIINNNGIGGGPDTLDPTRVPPSAYTPNAHYEKMAEIYGGKGYFVTEPSQLRPALEEAIKADKPAIVNIMISATSQRKPQQFAWLTR

SEQ ID NO: 4 - protein, 574 aa, synthetic construct
MTTIDGSEVIAESLARLGVKTVFGIVGIPVVEVADALINKGIKFIGFRNEQAASYAASVYGYLTQQPGVLLVVGGPGLVHALAGIYNSQSNKWPLLVLAGSSSSSEIYRGGFQELDQVSLMTSTFAKFSAKPPSISRVPELITKAFRLSISGKPGPTYIDLPADIIQSKIDSTDGVKYLQSVIPYTIEDIPKSVAPVNKLRQAVELIKSAQYPLLVVGKGASNCPRAVRNFVAEHMIPFLPTPMGKGVVPDSSEFNVSSARSDALRHADVIILAGARLNWMLHHGDFPKFKKNVKFIQIDLDSDEFGDNSNDSLKYGLYGDIGLTIESLNIALGKEHLVNSMLPVIETAKLKNIKKLELKGSVTPEQSESLMNHNQALTIITDSLGLKYDDTVFVSEGANTMDISRVVIPINYPKQRLDAGTNATMGVGLGYAIAAKAASPEKLVIAIEGDSAFGFSAMEIETAIRSDLPLFIIVLNNSGIYRGVSDVEKYAPFTNKPLPSTALSYKTRYDELGNSLGAVGMLVNNANELKLKMKECLDLYFNENKTIVLNVLIQSGAGTKLEFGWQNKPKSKL

SEQ ID NO: 5 - protein, 563 aa, synthetic construct
MSQEQLTGSSILAKSLKSLGVDVIFGIVGVPVVEVAEACIAEGIRFIGCRNEQSASFAAGAWGYLNKRPGVCLTVSGPGVVNAISGLYNAQANCWPMILIGGSCETNQIGMGAFQELDQVDACRNYTKFSGKCADLETIPFIVNKAYQVSKAGRPGPTYVDLPADLIQATTSKLPKLPEPFETPYCLPHTKDLSAAIEILKNSKRPLLVVGKGATYSRCENELKALVEEFNVPFLPTPMAKGILPDNHSLNAGSARSLALRKADVIVLLGARLNWMMQFGNRLNPQTKIIHVDISPEEFNINKKIDIGLFGNIPETIELIHQGLKKSGKSYSWIHFKNELQPNIEKNQEKLQKFLTAPLSPLMNHQQALNTVEEVLSKQFNGDYFLVSEGARTMDVTRMLVSSHLPRRRLDAGTLGVMGIGLGYALAGQLTHPDKKVVAIMGDSAFGFSAMEIETAARCKLPLIIIIINNNGIYHGLDDIKSVPSDKLPSFTLMPETRYDLLANSVYGQGFLVKDSTQLQSALQKCFNFDGVSIVNVMIDHRPASEGLYWLTREFSPAGQSKL

SEQ ID NO: 6 - protein, 559 aa, synthetic construct
MPEGPVAEIDGQTIIARALKQQGVEAMFGVVGIPVTGIAAAAQREGIKYVGMRHEMPATYAAQAVSYLGGRLGTALAVSGPGVLNAVAAFANAWSNRWPMILIGGSYEQTGHLMGFFQEADQLSALKPYAKYAERVERLERIPIYVAEAVKKALHGVPGPAYLELPGDIITAKIDESKVEWAPRVPDPKRTLSDPADVEAAIAALKTAQQPLIIVGKGVAASRAEVEIRAFVEKTGIPYLAMPMAKGLIPDDHDQSAAAARSFVLQNADLIFLVGARLNWMLHFGLPPRFRPDVRVVQLDFNPEEIGINVPTEVGMIGDAKATLSQLLDVLDRDGWRFPDDSEWVTAVSAEARQNAEAVQAMMQEDTQPLGYYRALRSIDERLPKDAIFVAEGASTMDISRTVINQYLPRTRLDAGSFGSMGLGHGFAIGAATQFPGKRVICLQGDGAFGFAGTECEVAVRYNLPITWIVFNNGGIGGHRAELFERDQKPVGGMSLGARYDILMQGLGGAAFNATNSDELDAAIEAALKIDGPSLINVPLDPDAKRKPQKFGWLTRTNE

SEQ ID NO: 7 - protein, 551 aa, synthetic construct
MAELTGAQIVAKALKQQGVEYMFGVVGIPVVPIAVHAQREGIKFFGFRNEQAASYAAAAIGYLTGRPGVCLAVSGPGMVHGIAGMANAWANCWPMILIGGANDSYQNGQGAFQEAPQIETARPYAKYAARPDSTRRIPFFVEQAVRATIYGRPGAAYLDLPGDLITGSVDESEVHFPPRCPDPPRTLAPWENIERALEALKSAERPLVIVGKGAAYARAEEEVRKFIDATQLPFLPTPMGKGVVPDDHPLAISPARSFALQNADVVLLLGARLNWILHFGLPPRFDPKVRVIQVDIAAEEIGNNVPAEVALVGDAKAIVGQMNEALTRAPWQYPAETTWWTGLRKKIEENAATVAEMMADESVPMGYYRVYRDIREYIPRDAIIVNEGANTMDIGRTLMPNFYPRHRLDAGSFGTMGVGVGQAIAAAAVHPDKRVFCIEGDSAFGFSGMEVETAARYGLNNIVFIIINNNGIGGGPDELDPTRVPPSAYTPNAHYEKMAEIYGGKGFFVTQPSELRPALEAALACDKPAIVNIMISARSQRKPQQFAWLTR

SEQ ID NO: 8 - protein, 551 aa, synthetic construct
MAELTGAQIVAKALKQQGVEYMFGVVGIPVVPIAVHAQREGIKFFGFRNEQAASYAAAAIGYLTGRPGVCLAVSGPGMVHGIAGMANAWANCWPMILIGGANDSYQNGQGAFQEAPQIETARPYAKYAARPDSTRRIPFFVEQAVRATIYGRPGAAYLDLPGDLITGTVDESEVHFPPRCPDPPRTLAPWENIERALDALKSAERPLVIVGKGAAYARAEEEVRTFIDMTQLPFLPTPMGKGVVPDDHPLAISPARSFALQNADVVLLLGARLNWILHFGLPPRFDPKVRVIQVDIAAEEIGNNVPAEVALVGDAKAIVEQMNEALSRAPWQYPAETTWWTGLRKKIEENAATVAEMMADESVPMGYYRVYRDIREYIPRDAIIVNEGASTMDIGRTLMPNFFPRHRLDAGSFGTMGVGLGQAIAAAAVHPDKRVFCIEGDSAFGFSGMEVETAARYGLNNIVFIIINNNGIGGGPDELDPTRVPPSAYTPNAHYEKMAEIYGGKGFFVTQPSELRPALEAALACDKPAIVNIMISARSQRKPQQFAWLTR

SEQ ID NO: 9 - protein, 550 aa, synthetic construct
MTTLDGATLIARSLRQQGVDYMFGIVGIPVVPVAIAFQREGGKFFGMRNEQAASYAAGAVGYLTGRPGACLAVSGPGMVHAIAGLANAWANGWPMILLGGANDSYQNGQGAFQEAPQIEAARPFAKYCARPDSTRRIPFFIEQAVRYSIYGRPGPVYVDLPGDIITGTAEESEVRFPPRCPDPPRALAPEENVRAALELLKQAERPLVIVGKGMAYARAEDEVREFIDRTRLPYLPTPMGKGVIPDDHPFSVAPARSFALQNADVVFLMGARLNWILHFGLPPRFAPTVKTIQLDIEPEEIGNNVPCTVPLVGDGKAIVGQLNAVLRGEPWEYPSETTWWTALRQKAAENEEMVRQMEQDDSVPMGYYRVLREVRELLPKDAIVASEGANTMDISRTVIPNYFPRHRLDAGTFGTMGVGLAQAIAAQVVHPDKKVVAIEGDSAFGFSGMEVEVMARYRLPITVIIVNNNGISGGPTQLDPNRVPPNAYLPNAHYEKIAEAFGGKGWFVTTPQELRPALEAALNSDTFSIVNIMIDTRAGRKPQQFAWLTR

SEQ ID NO: 10 - protein, 551 aa, synthetic construct
MATINGATLLARSLKQQGVEYMFGIVGFPVQPIAGAAQREGITFIGMRNEQAASYAAHAAGYLTGRPQACLVVSGPGVVHALAGLANAQSNCWPMILIGGASPTYQNGMGAFQEAPQVKLAEPYCKYAHAVEQVDRIPYYVEQAVRSSIYGRPGATYLDMPDDIIRAEIEEEKVEAKNTVPPPPRTQALDEDVESAVAALKSAERPLVIVGKGMAWSRAENEVREFIERSQLPFLATPMGKGVMPDDHPLSVGAARSFVLQNADVVFLLGARLNWILHYGLPPRYSPNVRVVQLDIAPEEIGANVPAEVGLVGDGKAVMRQVNRVLESSPWQYPSETTWRSGIANKIAENRVSTEAMMADDSSPMNYYHVLSTIRDMIPRDTIIASEGANTMDIGRTILNNYEPRTRLDAGTFGTMGVGLGFAIAASVTNPTKRIIDVEGDSAFGFSGMEVETACRHKMPITFIIINNNGIGGGPTEFDTSKPLPPNAYTPSAHYEKMMDAFGGKGYFVTESSELKPALEAALNTDGPSLVNIMISNRATRKPQEFRWLTT

SEQ ID NO: 11 - protein, 570 aa, synthetic construct
MTDTTPATADTTNGAAAGETILGGVLLVRSLKQQGVDYMFGVVGFPVSELAGYAQDEGIKYIGMRNEQAASYAAQAASYILGRPQACIVVSGPGVIHGLAGLANAKSNCWPMILIGGASAVSQNGMGAFQEENQVEIARLVSKYAHSLDRVDRIPYYVEQAVRTSLYGRPGPAYLDAPDDILTAEIPLSQIKTVPTVPDPPRPGVPERDIKAAVAALKSAERPLVIVGKGMAWSRAENEVLEFIEKTQIPFLPTPMGKGVVDDDHPLAISPARTLALREADVVLLLGARLNWILHFGKPPRWAEDVRIIQVDIAAEEIGANVPAEVGLVGDGAAIVAQLNQALDEDGWQYPGETTWRSALKAKVDENVAVSAQLMADDSVPMNYYHPLQAIRDTLPEDTIIVSEGAGTMDIGRTVLPNHGPRTRLDAGTYGTMGIGLGFAIGAAIAKPGTRIVDVEGDAAFGFSGMEYETMVRHNLPITIVVINNNGIGGGVAELPEDRDPPPGVYLPSARYERIADMFGGRSYYVTQPEELEPALREANTGEGPAIVHIRIDPSAGRKPQQFGWHTPTN

SEQ ID NO: 12 - protein, 479 aa, synthetic construct
MDKDLLSVQQVRDLVKACKAAQKKYVEFSQEKMDKIVHEMSMEVRQYDEKLAKLAVEETGFGKWEDKVIKNRFASTYIYDFIKNMKTVGILREENEVMEVGVPVGVIAGLIPSTNPTSTTIYKILISLKAGNGIVISPHPNAKNCIIETANILKRAAIKAGAPEGLIGVIEIPTIQATDALMKHDDVSLILATGGEAMVRAAYSSGTPAIGVGPGNGPAFIERSANVKMAVKRIMQSKTFDNGTICASEQSIIAEACNRTEIMKEVENQGGYFMPREDADKLARFILRPNGTMNPAIVGKSAEVIANLAGIKIPLGTRVLLSEETTVSNSNPYSSEKLAPILAFYVEDNWEKACERSIEILNHEGRGHTMIIHSEDREVIREFALKKPVSRLLVNTPGSLGGIGATTNLAPALTLGCGAVGGSSTSDNITPMNLINIRRVAWGVRELDYFRTENVEQTNVDSKDMEELIKKVLNEILNR

SEQ ID NO: 13 - protein, 483 aa, synthetic construct
MTTLDKDLASIQEVRNLLTEAKAAQESLAKMSQEQIDRICEAIAASAYEAREKLAKMAHQETGFGIWQDKVVKNSFASKFVWDSIKEMKTVGILNEDKEQKVIDVAVPVGVVAGLIPSTNPTSTVIYKALIAIKAGNAIVFSPHPNALQAILATVEIISKAAEKAGCPKGAIGCMLKPTMQGTAELMKHQYTSLILATGGSAMVKAAYSSGTPAIGVGPGNGPAYIEKSADIPLAVKRIMDSKTFDNGTICASEQSIIAETSNKAEVIAELKKQGAYFLSPEESAQLERYIMRPNGSMNPQIVGKSVQAIAELTHLSVPKEARVLIAEETKVGHKVPYSREKLAPILAFYTVGNWEEACELAMDILYHEGAGHTMMIHSQNDEVIRQFGLKKPVSRVLVNTPGALGGIGATTNLAPALTLGCGAVGGSSTSDNISPANLFNVRRIAYGIRELEDLREQPVSSSGFNEEQLVDTLVERILAKLQ

SEQ ID NO: 14 - protein, 487 aa, synthetic construct
MTLLDKDLRSIQEARELIGKAKAAQSQLALLSQEQIDRIVKAIAEAGYDNREELAKIAAVETGFGKWEDKVLKNAFASQAVYESLKDLKTIGILKEDMQQKVMEIGVPLGVIAALIPSTNPTSTTIYKAMISLKAGNAIIFSPHPNAINCILETVRVIKEAAVKAGCPSDAISCMSIPAIEGTETLMKHKDVSLILATGGSAMVKAAYSSGTPAIGVGPGNGPAFIERTANIPLAVKRIFDSKTFDNGVICASEQSIVVEECIREEVIEECSKQGGYFLSERERKQLEKFIMRSNGTMNPAIVGKSVEQIAKLAELNIPDGTRVLIAKESRVGRDVPYSREKLAPILAFYTEKDWQAACERCIQLLLNEGAGHTLIIHSENEEVIKQFALKKPVSRLLVNTPGALGGIGATTNLVPALTLGCGAVGGTSTSDNIGPLNLINIRRVAYGVKELEDLRENTPTCEPSFGVCDQKELIESIVKQVLAQLH

SEQ ID NO: 15 - protein, 489 aa, synthetic construct
MMEMDKDLQSIHEARTLIGQAKEAQRQLAKLGQEDIDHIVKAMAEAAYEHRERLAKLAVEDTGFGIVKDKVLKNLFASYGVYRAIKDMKTVGIINEDEQEKIVEVAVPVGVIAALVPSTNPTSTVMNKALIAIKAGNAVVFSPHPSALNCILETTRILAEAAEAAGCPKGAITSMTKPTMQGTDTLMKHRDVSLILATGGSAMVKAAYSSGTPAIGVGPGNGPAFIERSANVKQAVKRIIDSKTFDNGVICASEQSVIVEADHKEVVVEEFKRQHAYFLSKEEAAKLEKFIMRPNGTMNPQIVGKSALFLADLAGISVPSNTRVLIAEEDKVGKDVPFSREKLSPILAFYIEKDWRAALDRSIEILLNEGAGHTMTVHSEKEEIIRAFTLEVPVFRLLVNTSATLGAIGATTNLLPAYTLGCGALGNGSTSDNVGPMNLLNIKRVAIGIKDLAEIESESNNTKLSSAELNEDMVERVVEQVLRQLYVMS

SEQ ID NO: 16 - protein, 490 aa, synthetic construct
MEMLDKDLRSIQEVRDLIKKAKEAQAKLAVMTQAQIDAIVKAIADAGYAHREKLAKMANEETGFGRWEDKIVKNAFASKHVYESIKDMKTVGIINDDKAHKVMDVAVPVGVVAGLIPSTNPTSTVIYKALISLKAGNSIVFSPHPNALKSILETVKVINDAAVQAGCPEGAIASMTVPTIQGTDQLMKHKDTSVILATGGEAMVKAAYSSGTPAIGVGPGNGPAFIEKSANFELAVKRILDSKTFDNGTICASEQSVIVEACSKEAVMAEFKKQGAYFLTAEEAVQLGKFIMRANGTMNPQIVGRSVDHIAKLANLNVPAGTRVLIAEETSVGRNVPYSREKLAPILAFYTEDNWEAACARSIEILNGEGAGHTMMIHSENEEIIRQFALKQPVSRLLVNTPGALGGIGATTAIAPALTLGCGAVGGSSTSDNVSPMNLLNIRKLTYGLRELEDLVEQPTTQAAPAAATISQDDKEQLISMIVARILEKM

SEQ ID NO: 17 - protein, 497 aa, synthetic construct
MYRDRVRLPSLLDKVMSAAEAADLIQDGMTVGMSGFTRAGEAKAVPQALAMRAKERPLRISLMTGASLGNDLDKQLTEAGVLARRMPFQVDSTLRKAINAGEVMFIDQHLSETVEQLRNHQLKLPDIAVIEAAAITEQGHIVPTTSVGNSASFAIFAKQVIVEINLAHSTNLEGLHDIYIPTYRPTRTPIPLTRVDDRIGSTAIPIPPEKIVAIVINDQPDSPSTVLPPDGETQAIANHLIDFFKREVDAGRMSNSLGPLQAGIGSIANAVMCGLIESPFENLTMYSEVLQDSTFDLIDAGKLRFASGSSITLSPRRNADVFGNLERYKDKLVLRPQEISNHPEVVRRLGIIGINTALEFDIYGNVNSTHVGGTKMMNGIGGSGDFARNAHLAIFVTKSIAKGGNISSVVPMVSHVDHTEHDVDILVTEQGLADLRGLAPRERARVIIENCVHPSYQAPLLDYFEAACAKGGHTPHLLREALAWHLNLEERGHMLAG

SEQ ID NO: 18 - protein, 533 aa, synthetic construct
MSTSDVLNPEEVALVLREKVSPILRGHGGDLVLSHIRGKSIYIRFTGACRGCPAALETAERTVQAVLREHFGDEDIDAVLDNGVSEDLINQAKQILQKSKKIMNEILAQYKSKIVSADDAVKVIKNGERVSLSHAAGVPQVCVDALVRNAEHFQGVEIYHMLCLGEGKYMLPEMAPHFRHVTNFVGGNSRQAVAENRADFIPAFFYEVPTLFRKGILPIDVAIVQLSMPDAEGYCSFGVSSDYTKPSTEVARVVIGEINAQTPYVHGDNKIHISKLDYIVLADYPLYTIPKAPIGPVEEAIGRNCAELVEDGSTLQLGIGAIPDAALLFLKDKKDLGIHTEMFADGVIELVRAGVITGKKKSLHPGKMVATFLMGTEEVYKFAHNNPDVELYPVDYVNDPRTVAMNDNMVSINSCIEVDLMGQVVSETIGPKQFSGTGGQVDYVRGATWSKNGKSIMAMPSTARKGAASRIVPMIAEGASVTTLRNDVDYVVTEYGIARLKGRSLRQRAEALISIAHPDFREELMKVYRERFE

SEQ ID NO: 19 - protein, 414 aa, synthetic construct
MNWGLNMKVLVINAGSSSLKYQLIDMINESPLAVGLCERVGIDNSIITQKRFDGKKLEKQVDLPTHRVALEEVVKALTDPEFGVITDMGEINAVGHRVVHGGEKFTTSALFDAGVEEAIRDCFDLAPLHNPPNMMGITACAEIMPGTPMVIVFDTAFHQTMPAYAYMYALPYDLYEKYGVRKYGFHGTSHKYVAGRAALMLGKPIEDTKIITCHLGNGSSIAAVKGGKSIDTSMGFTPLEGVAMGTRCGSIDPAVVPFIMDKEGLSSREIDTLMNKKSGVLGVSGISNDFRDLDEAASHGNERAELALEIFAYSVKRVIGEYLAVLNGADAIVFTAGIGENSASIRKRILAGLDGLGIKIDEEKNKIRGQEIDISTPDSSVRVFVIPTNEELAIARETKEIVETEAKLRSSVPV

SEQ ID NO: 20 - protein, 333 aa, synthetic construct
MVTFLEKISERAKKLNKTIALPETDDIRTLQAAAKAIERGVANIVLIGDEAKIKELAGDLDLSKAKIVNPETYEKKDEYIQAFYELRKHKGITLESAAEVMKDYVYFAVMAAKLDEVDGVVSGAVHSSSDTLRPAVQIVKTAPGAALASAFFIIAVPDCEYGSDGTFLFADSGMVEMPSVEDVANIAVISAKTFELLVQDDPYVAMLSYSTKGSAHSKLTEATIASTKLAQELAPDIPIDGELQVDAAIVPKVAASKAPGSPVAGKANVFIFPDLNAGNIAYKIAQRLAKAEAYGPITQGLAKPINDLSRGCSDEDIVGAIAITCVQAAAQDK

SEQ ID NO: 21 - protein, 399 aa, synthetic construct
MKILVVNAGSSSLKYQLFDMDTESVIVKGGVERIGIRGSVLHHKWAQGEKVIEQDMPNHKVAMQAVLDALVHPEYGAIHSMSEIDAVGHRVLHSGGDFDGSVLLDDEVLKICKKNAELGPLHMPANILGIEACREVMPHTPMALVFDTAFHATMPPHAYMYAVDYDDYKNYKVRKYGFHGTSHKYVSQEAIKYLGRGAAGTKIITAHLGGGSSLSAVMDGKCVDTSMGFTPLAGVPMGTRSGDIDPAVLEFLAAKKGYTVLDCINYLNKQCGVAGISGISSDFRDLTKAAAEGNERAQLALDMFAYAVKKYIGSYIAAMDGLDCLVFTAGIGENTWQVREMICDKMDCFGIALDAEKNRLKNDGAIHDITGEGSKVKVLVIPTNEELVIARETKELVEA

SEQ ID NO: 22 - protein, 358 aa, synthetic construct
MADFFNKVKDKMSAVKDKLGEMIEKEEDTFLYRIKKRASELNKRIVLCEGEDSRVVKAASVAAKQGVAKIVLLGNAEQIAKDNPDIDLSAVEIVDPAASEKRAEYAALLYQLRQAKGMTQEEAEKLSYDNTYFGVLMVKAGDADGLVSGACHSTANTLRPGLQIVKAAPGVPLVSSCFFMVAPPAGNQYCEDGVFIYSDCGLNENPNSEQLAEIAIISAKTAEKIAGLEPRVAMLSFSTKGSAKHADIDKVTAAYRIAKEKAPDLALDGELQLDSAIVPAVAKSKAPGSKVAGHANVLIFPDLDAGNIGYKLTERLGGFMAVGPVCQGFAKPINDLSRGCKWEDIVATIAITALQTQM

SEQ ID NO: 23 - protein, 401 aa, synthetic construct
MKILVINCGSSSLKYQLINMEDKGVLAQGLVERIGISGSILTQKVDGRDKYVIESPLKDHQEAIDLVLRTLVDDNQGVIKSMEEISAVGHRVVHGGEKYATSVVVTEEVIKNLEDFIKLAPLHNPPNIIGIRACQALMPNTPMVAVFDTAFHQTMPEKAFMYPLPYELYKEDHIRRYGFHGTSHKYVAGEVAKWMKKDIKDIKTITCHLGNGVSVTAVNGGQSIDTTMGFTPLDGIIMGSRSGSIDPAIVTYLVKEKGYSIDEVNEILNKKSGVLGISGLGTDFRDIRAAVEERNDKRALLTMDIYGYQIKKQIGAYAAAMAGVDAIVFTAGIGEHAPEIRVRALTDMEFLGIELDVDKNDNQNIGDGMEISKPSSKVKVFVIPTNEELMIAEETLELIQK

SEQ ID NO: 24 - protein, 333 aa, synthetic construct
MNLMQKIWDAAKSDKKKIVLPEGNEERTIVAAEKINRLGLAHPILIGNKEEIINKGHALDVDLSQVEIIDPAESENLEKYITAFYELRKNKGITLEKAEKIVKDPLYFATMMVKLDDADGMVSGAVHTTGDLLRPGLQIIKTAPGVSVVSSFFIMEVPNSSYGEDGLLLFADCAVNPMPNEDQLAAIAIATAETAKRLCNMDPKVAMLSFSTKGSADHEVVDKVRNATKKANELRPDLDIDGELQLDASIVEKVANQKAPGSKVAGKANVLVFPDLQAGNIGYKLVQRFANAKAIGPVCQGFAKPINDLSRGCSSDDIIDVVALTAVQAQNIK

SEQ ID NO: 25 - protein, 14 aa, synthetic construct
ATADRKPQQFNWHG

SEQ ID NO: 26 - protein, 15 aa, synthetic construct
ATSQRKPQQFAWLTR

SEQ ID NO: 27 - protein, 14 aa, synthetic construct
QGSTRKAQQFAWHS

SEQ ID NO: 28 - protein, 14 aa, synthetic construct
QGSARKPQQFAWHS

SEQ ID NO: 29 - protein, 15 aa, synthetic construct
PTAKKAPQTYSHWSS

SEQ ID NO: 30 - protein, 14 aa, synthetic construct
QGSVRKPQQFRWHA

SEQ ID NO: 31 - protein, 15 aa, synthetic construct
PRAGRKPQQFGWLTT

SEQ ID NO: 32 - protein, 15 aa, synthetic construct
PGAGRRQQQFAWLAR

SEQ ID NO: 33 - protein, 15 aa, synthetic construct
PMADRKXQEFAWNRR

SEQ ID NO: 34 - protein, 15 aa, synthetic construct
ADAKRKPQKFGWLTS

SEQ ID NO: 35 - protein, 15 aa, synthetic construct
PRAMRRKQEFGWLTR

SEQ ID NO: 36 - protein, 15 aa, synthetic construct
PQARAKPQKHHWLTA

SEQ ID NO: 37 - protein, 15 aa, synthetic construct
PRAGRKPQKFSWLTR

SEQ ID NO: 38 - protein, 17 aa, synthetic construct
PNGGRRPQPQEFAWLTR

SEQ ID NO: 39 - protein, 16 aa, synthetic construct
DAASPDSGKGLGFVPD

SEQ ID NO: 40 - protein, 14 aa, synthetic construct
PFAGAESGRLQHKN

SEQ ID NO: 41 - protein, 12 aa, synthetic construct
PSGTTPKILTKH

SEQ ID NO: 42 - protein, 14 aa, synthetic construct
YAGKESGNIGALNP

SEQ ID NO: 43 - protein, 15 aa, synthetic construct
PSAGTESGHISHLNP

SEQ ID NO: 44 - protein, 15 aa, synthetic construct
ESMGKESGHIGNLNP

SEQ ID NO: 45 - protein, 15 aa, synthetic construct
TAAGTESGHIANLNP

SEQ ID NO: 46 - protein, 15 aa, synthetic construct
PHVGTESGHLSKLNP

SEQ ID NO: 47 - protein, 15 aa, synthetic construct
EASGTESGRIGNLNP

SEQ ID NO: 48 - protein, 16 aa, synthetic construct
MEIAPSRDGRAGALMG

SEQ ID NO: 49 - protein, 15 aa, synthetic construct
HKARAQTVRFSAYST

SEQ ID NO: 50 - protein, 15 aa, synthetic construct
PRARSQTVRFSTYQP

SEQ ID NO: 51 - protein, 14 aa, synthetic construct
SGAGTKLEFGWQNK

SEQ ID NO: 52 - protein, 16 aa, synthetic construct
RPASEGLYWLTREFSP

SEQ ID NO: 53 - protein, 14 aa, synthetic construct
PGGTKKLEFNWMKK

SEQ ID NO: 54 - protein, 14 aa, synthetic construct
PGGRKKLSFGWMNK

SEQ ID NO: 55 - protein, 15 aa, synthetic construct
PFAGVESGNVHAFNA

SEQ ID NO: 56 - protein, 14 aa, synthetic construct
PYAGAESGRMQHRN

SEQ ID NO: 57 - protein, 15 aa, synthetic construct
PAAGSESGNIGSLNP

SEQ ID NO: 58 - protein, 15 aa, synthetic construct
PAAGSEGGNIGSLNP

SEQ ID NO: 59 - protein, 14 aa, synthetic construct
PGGRTKLEFGWMNK

SEQ ID NO: 60 - protein, 350 aa, synthetic construct
QSAKRPLVIVGKGMAWSRAENEVRQFIERTRLPFLATPMGKGVMPDDHPLSVGGARSHALQEADLVFLLGARFNWILHFGLPPRYSKDVRVIQLDLSAEEIGNNRQAEVALVGDGKAIVGQLNQALSSRQWFYPAETPWREAIAAKIAGNQAAVAPMIADNTSPMNYYRVYRDIAARLPRNAIIVGEGANTMDIGRTQMPNFEPRSRLDAGSYGTMGIGLGFAVAAAAVHPGRPVIAVQGDSAFGFSGMEFETAARYGMPIKVIILNNGGIGMGSPAPRDGQPGMPHALSHDARYERIAEAFGGAGFYVTDSAELGPALDAAMAFKGPAIVNIKIAATADRKPQQFNWHG

SEQ ID NO: 61 - protein, 350 aa, synthetic construct
KSAERPLVIVGKGAAYSRAENEVREFLETTQLPYLASPMGKGVMPDDHPLSIAPARSAALLGADVILLMGARLNWMMHFGHPPRFDPKVRVIQMDISAEEIGTNVPTEVALVGDAKAITTQLNASLKQQPWQYPSETTWWTGLRKKIDENGATVAEMMADESVPMSYYRVYREIRDLIPNDAIIQNEGASTMDIGRTLMPNFLPRHRLDAGSFGTMGVGLGQAIAAAAVHPDKHVFCIEGDSAFGFSGMEVETAARYGMKNITFIIINNNGIGGGPDTLDPTRVPPSAYTPNAHYEKMAEIYGGKGYFVTEPSQLRPALEEAIKADKPAIVNIMISATSQRKPQQFAWLT

SEQ ID NO: 62 - protein, 8 aa, synthetic construct
  VAR_SEQ 7: note = X= any amino acid, may repeat
RKPQQFXW

(19) United States
(12) Patent Application Publication
(10) Pub. No.: US 2026/0078416 A1
(43) Pub. Date: Mar. 19, 2026

(54) ORTHOGONAL METABOLIC FRAMEWORK FOR ONE-CARBON UTILIZATION

(71) Applicant: Mojia Biotech Pte. Ltd., Singapore (SG)

(72) Inventors: Ramon Gonzalez, Tampa, FL (US); Alexander Chou, Houston, TX (US); James MacAllister Clomburg, Houston, TX (US); Fayin Zhu, Tampa, FL (US); Seung Hwan Lee, Tampa, FL (US); Mohammadreza Nezamirad, Tampa, FL (US)

(21) Appl. No.: 18/442,550

(22) PCT Filed: Aug. 16, 2022
(86) PCT No.: PCT/US2022/040516

§ 371 Date: Sep. 18, 2025

Related U.S. Application Data
(60) Provisional application No. 63/233,589, filed on Aug. 16, 2021.

(51) Int. Cl. C12P 7/42; C12N 9/00; C12N 9/10; C12N 9/88; C12N 15/70; C12P 7/56

(52) U.S. Cl. CPC C12P 7/42; C12N 9/1025; C12N 9/1029; C12N 9/88; C12N 9/93; C12N 15/70; C12P 7/56; C12Y 203/01023; C12Y 401/01008; C12Y 602/01003; C12N 2800/101

(57) ABSTRACT

Provided are systems and methods for converting C1 substrates to products contain more than one carbon, without producing central metabolic building blocks as intermediate products. In an embodiment, system/method can include a biochemical pathway enabling an orthogonal platform for C1 utilization based on formyl-CoA elongation (FORCE) reactions. In an embodiment, the system/method can include acyloin condensations between formyl-CoA and carbonyl-containing molecules. In an embodiment, the system/method can include a reactions catalyzed by the enzyme 2-hydroxyacyl-CoA lyase (HACL).

### CROSS REFERENCE TO RELATED APPLICATIONS

This application is a continuation of International Application No. PCT/US22/040516, which designated the United States and was filed on Aug. 16, 2022, published in English, which claims priority to U.S. Provisional Patent Application 63/233,589 filed Aug. 16, 2021. The entire teachings of the above applications are incorporated herein by reference.

### STATEMENT REGARDING FEDERALLY SPONSORED RESEARCH OR DEVELOPMENT

This invention was made with government support under Award No. DE-AR0001508 and DE-EE0008499 awarded by the Department of Energy and Joint Genome Institute. The government has certain rights in the invention.

### SEQUENCE LISTING

The sequence listing submitted via EFS, in compliance with 37 CFR § 1.52(e)(5), is incorporated herein by reference. The sequence listing XML file submitted via EFS contains the file “43673005US1SeqList.xml”, created on Oct. 16, 2025, which is 67,715 bytes in size.

### BACKGROUND

### 1. Field of the Disclosure

The present disclosure relates to the field of bioconversion of carbon-containing substrates into products.

### 2. Description of the Background

Metabolism is one of the conserved features of all living cells that has evolved to optimize an organism's survival and reproductive capabilities. All organisms, from single-celled bacteria to those as complex as humans, display a canonical architecture of metabolism that has evolved with central metabolism serving as a link between catabolic (degradation) and anabolic (synthesis) pathways through which carbon and energy sources are utilized for biosynthesis of cellular components and metabolic products. Often referred to as a “bow-tie” or an “hourglass” architecture, this structure can be exemplified by the metabolism of one-carbon (“C1”) substrates.

However, the robustness and plasticity possessed by most poses substantial engineering challenges. For example, out of the necessity to generate and balance the use of precursor metabolites, many cells have evolved intricate regulatory mechanisms to control pathway fluxes. Furthermore, as metabolic pathways were shaped by evolution and a given fitness advantage, opposed to maximizing the utilization or synthesis of a desired compound, native pathways often limit the attainable product titers, rates, and yields due to intrinsic metabolic inefficiencies and negative cross talk between product-forming and growth-sustaining reactions. This is again epitomized by C1 substrates, as current approaches toward biological product synthesis from these molecules involving both natural and synthetic pathways must concurrently engineer C1 assimilation, central metabolism, and product synthesis pathways for effective production of target chemicals. As such, while C1 molecules can be utilized by microorganisms for growth, the biological production of industrial chemicals from C1 feedstocks as well as the efficient growth of industrial organisms on C1 substrates remain as open challenges, despite the incentive and opportunity for such processes. To date, attempts to engineer C1 bioconversion, even those exploiting synthetic pathways or novel enzyme designs, have relied on central carbon metabolism for utilizing various C1 substrates as a carbon source and/or for product synthesis. These designs have required rewiring a host's metabolic network to accommodate C1 bioconversion, which has proven challenging.

### SUMMARY

The generation of products, such as industrial chemicals, from C1 substrates may be facilitated via the use of orthogonal biological systems. Orthogonal systems are less likely to impact or be impacted by their biological context, enabling more predictable and consistent behavior compared to modifying or repurposing natural parts. An orthogonal metabolic paradigm could be a powerful alternative to engineering within the bow-tie architecture, as pathways to target products bypassing central metabolism could be both more direct (i.e., fewer steps) and circumvent many intrinsic regulatory mechanisms and carbon and energy inefficiencies.

Specific to C1 substrates, as they do not have any pre-existing carbon-carbon bonds, there is in principle no reason to first produce central metabolic building blocks on the way to the desired product. An opportunity therefore exists to establish an orthogonal metabolic framework based on the direct synthesis of products from C1 molecules, provided the right biochemistry.

The present disclosure relates to the conceptualization and design of biochemical pathways enabling an orthogonal platform for C1 utilization based on formyl-CoA elongation (FORCE) reactions. This approach relies on acyloin condensations between formyl-CoA and carbonyl-containing molecules. These reactions can be catalyzed by the enzyme 2-hydroxyacyl-CoA lyase (HACL). HACL (and the related enzyme oxalyl-CoA decarboxylase, OXC17) can utilize carbonyl-containing acceptors of broad chain length and functionalization, including the C1 compound formaldehyde, to generate acyl-CoAs amenable to a wide-range of biochemical conversion. In addition to product synthesis, FORCE pathways can serve as the basis to enable growth on non-native C1 substrates via the generation of multi-carbon compounds that are native substrates to the host microorganism.

In one aspect of the present disclosure, a recombinant 2-hydroxyacyl-CoA synthase enzymatically capable of least 2-fold, alternatively 3-fold greater rate of formation of a 2-hydroxyacyl-CoA from a carbonyl-containing compound and formyl-CoA compared to the Rhodospiralles bacterium URHD0017 2-hydroxyacyl-CoA synthase.

In some embodiments, the carbonyl-containing compound is selected from the group consisting of an aldehyde and a ketone. In some embodiments the 2-hydroxyacyl-CoA synthase is selected from Table 1 or table 2. In some embodiments the 2-hydroxyacyl-CoA synthase has 90% or greater identity to the enzyme in Table 1 or table 2.

Another aspect of the present disclosure the enzyme further comprising producing the formyl-CoA by contacting a one carbon substrate with an enzyme catalyst. In another aspect, the enzyme further comprising converting of a substrate to a carbonyl-containing compound by contacting the substrate with an enzyme catalyst. In another aspect, the enzyme further comprising converting the 2-hydroxyacyl-CoA to an organic chemical product. In yet another aspect, the carbonyl-containing compound is an aldehyde with at least one substituent group.

In some embodiments the aldehyde substituent group may be a hydroxyl, a carbonyl, an alkyl or an amine. In some embodiments the carbonyl-containing compound is a ketone and the ketone is a methyl ketone.

In some embodiments the one carbon substrate is formaldehyde and the enzyme catalyst that produces formyl-CoA is an acyl-CoA reductase (acylating aldehyde dehydrogenase) that catalyzes the conversion of formaldehyde to formyl-CoA.

In some embodiments the one carbon substrate is methanol and the enzyme catalysts to produce formyl-CoA are a methanol dehydrogenase catalyzing the conversion of methanol to formaldehyde; and an acyl-CoA reductase (acylating aldehyde dehydrogenase) catalyzing the conversion of formaldehyde to formyl-CoA.

In some embodiments the one carbon substrate is methane and the enzyme catalysts to produce formyl-CoA are a methane monooxygenase catalyzing the conversion of methane to methanol; a methanol dehydrogenase catalyzing the conversion of methanol to formaldehyde; and an acyl-CoA reductase (acylating aldehyde dehydrogenase) catalyzing the conversion of formaldehyde to formyl-CoA.

In some embodiments, the one carbon substrate is formate and the enzyme catalysts to produce formyl-CoA are an acyl-CoA synthase catalyzing the conversion of formate to formyl-CoA or a formate kinase catalyzing the conversion of formate to formyl-phosphate and a phosphate formyl-transferase catalyzing the conversion of formyl-phosphate to formyl-CoA.

In some embodiments, the one carbon substrate is carbon dioxide and the enzyme catalysts to produce formyl-CoA are a carbon dioxide reductase catalyzing the conversion of carbon dioxide to formate; and an acyl-CoA synthase catalyzing the conversion of formate to formyl-CoA; or a formate kinase catalyzing the conversion of formate to formyl-phosphate and a phosphate formyl-transferase catalyzing the conversion of formyl-phosphate to formyl-CoA.

In some embodiments, the product derived from the 2-hydroxyacyl-CoA is an aldehyde and wherein the enzyme catalysts converting the 2-hydroxyacyl-CoA to said product is an acyl-CoA reductase catalyzing the conversion of the 2-hydroxyacyl-CoA to the aldehyde.

In some embodiments the product derived from the 2-hydroxyacyl-CoA is an alcohol and wherein the enzyme catalysts converting the 2-hydroxyacyl-CoA to said product are an acyl-CoA reductase catalyzing the conversion of the 2-hydroxyacyl-CoA to the aldehyde; and an alcohol dehydrogenase (aldehyde reductase) catalyzing the conversion of the aldehyde to the alcohol.

In some embodiments, the product derived from the 2-hydroxyacyl-CoA is a carboxylic acid and wherein the enzyme catalysts converting 2-hydroxyacyl-CoA to said product is a thioesterase catalyzing the conversion of the 2-hydroxyacyl-CoA to the carboxylic acid.

In some embodiments, the enzyme catalysts are contained in a recombinant microorganism harboring the genes for expressing each enzyme.

In some embodiments, the substrates are contacted with the recombinant microorganisms containing the enzyme catalysts in an aqueous media optionally containing buffers, salts, vitamins, minerals.

In some embodiments, the substrates are contacted with enzyme catalysts by addition of each to an aqueous reaction mixture optionally containing buffers, salts, vitamins, minerals, and cofactors.

In some embodiments, the enzyme catalysts are provided as crude cell extract. In some embodiments, the enzyme catalysts are provided as purified proteins.

One aspect of the present disclosure is a modified organism comprising at least one heterologous enzyme described herein, wherein the organism is capable of growth on a one-carbon substrate, and wherein the organism without the heterologous enzyme is incapable of growth on a one-carbon substrate.

In some embodiments, the modified organism is a bacteria. In some embodiments, the organism comprises two or more modified enzymes.

### BRIEF DESCRIPTION OF THE DRAWINGS

FIG. 1 is a diagram of, and flow-chart for, the canonical (a) and synthetic (b, c) metabolic architectures for biological C1 utilization;

FIG. 2 is a diagram of, and flow-chart for, FORCE pathways for product synthesis from C1 substrates;

FIG. 3 shows graphs of an analysis of FORCE pathways;

FIG. 4 shows graphs of In vitro assessment of core module of the FORCE pathway using purified enzymes;

FIG. 5 shows graphs of certain cell-free prototyping of the α-reduction variant of the FORCE product synthesis pathway;

FIG. 6 shows a diagram and graphs of resting cell bioconversions of C1 substrate formaldehyde using the aldose elongation and α-reduction variants of the FORCE pathways;

FIG. 7 shows a diagram and graphs of FORCE pathway implementation in growing cell cultures using methanol as the C1 substrate;

FIG. 8 shows diagrams of simulated flux maps from genome scale E. coli models for growth using FORCE pathways variants: a) (form) aldehyde elongation, b) α-reduction, c) aldose elongation;

FIG. 9 shows a diagram of, and graphs for, two-strain system for evaluating the ability of FORCE pathways to enable growth on C1 substrates;

FIG. 10 is a diagram of a consolidated illustration of the orthogonal C1 pathway concept;

FIG. 11 is a diagram of an alternative FORCE pathway based on dehydration of the 2-hydroxyacyl-CoA and α-reduction;

FIG. 12 is a graph of the impact of NADH/NAD+ ratio on formaldehyde (top) and methanol (bottom) conversion to glycolate or acetate via FORCE pathways;

FIG. 13 is a graph of the impact of termination on the iterative aldose elongation pathway;

FIG. 14 is a graph of the production of glycolate from formate by E. coli engineered with a formate-activating pathway;

FIG. 15 is a graph of the paraformaldehyde solubilization rate and resting cell bioconversion with paraformaldehyde;

FIG. 16 shows images of profiles for glycolate, formate, and formaldehyde concentration and a graph of cell-growth of the sensor strain in the two-strain system with 5 mM paraformaldehyde;

FIG. 17 shows images of profiles for glycolate, formate, and formaldehyde concentration and a graph of cell-growth of the sensor strain in the two-strain system with 500 mM methanol;

FIG. 18 shows images of profiles for glycolate, and formaldehyde concentration in the two-strain system with 1 mM formaldehyde and 10 mM formate;

FIG. 19 depicts bioprospecting strategy used for identification of 34 2-hydroxyacyl-CoA synthase (HACS) variants with Rhodospirillales bacterium URHD0017 (RuHACL) as a starting reference gene;

FIG. 20 depicts (a) Glycolate production from formaldehyde via 2-hydroxyacyl-CoA synthase (HACS) and acyl-CoA reductase (LmACR) activity (b) High throughput resting-cell bioconversion platform for screening HACS variants (c) Screening result of initial 29 HACS variants and RuHACL on glycolate productivity per cell density (uM/OD) and level of expression;

FIG. 21 depicts (a) Glycolate production from formaldehyde via 2-hydroxyacyl-CoA synthase (HACS) and acyl-CoA reductase (LmACR) activity. 5 mM formaldehyde is used as the sole carbon source. (b) Two inducible promoter, HACS under control of IPTG-inducible T7 promoter and LmACR and EcAldA under control of cumate-inducible T5 promoter are used for analysis. (c) Glycolate productivity (uM/OD) using RuHACL^{G390N} as the HACS with changing IPTG and cumate concentrations (d) Glycolate productivity (uM/OD) using JGI15 as the HACS with changing IPTG and cumate concentrations;

FIG. 22 depicts (a) Glycolate production from co-feeding formaldehyde (0.5 mM or 5 mM) and formate (20 mM). Expression of HACS and FAE (AbfT) are controlled independently by inducible promoters (IPTG and cumate) (b) HACS screening results for the starting reference, RuHACL and the two best variants from initial round. C1-C1 condensation reaction activity is represented by glycolate productivity under 0.5 mM or 5 mM formaldehyde (FALD);

FIG. 23 depicts (a) Protein structure of JGI15 modeled using AlphaFold2. (b) Protein structure of JGI20 modeled using AlphaFold2. (c) Two ligands (thiamine diphosphate and formyl-CoA) bound to the active site via alignment with crystal structure of OfOXC (PDB code: 2JI8);

FIG. 24 depicts (a) Identification of active site residues by selecting amino acid residues within 3.5 Å from thiamine diphosphate (TPP). (b) Identification of active site residues by selecting amino acid residues within 3.5 Å from formyl-CoA (CoA). (c) Screening result of alanine scanning the active site residues represented by glycolate productivity (μM/OD/h) with respect to the wildtype JGI20 using formaldehyde (0.5 or 5 mM) and formate (20 mM) system with AbfT co-expression;

FIG. 25 depicts (a) Sequence analysis of first round JGI variants at the c-terminal end. Active variants (with asterisk) show fairly conserved RKPQQF-W (SEQ ID NO: 62) residues SEQ IDs in descending order are SEQ ID NO: 25 to SEQ ID NO: 59 (b) Screening result of alanine scanning the conserved c-terminal residues represented by glycolate productivity (μM/OD/h) with respect to the wildtype JGI20 using formaldehyde (0.5 or 5 mM) and formate (20 mM) system with AbfT co-expression;

FIG. 26 depicts (a) Alignment of JGI15 and JGI20 AlphaFold structure alignment represented by aligned amino acid residues. N461 of JGI20 and R493 of JGI15 are the two residues not aligned between the two variants JGI15 sequence on top is SEQ ID NO: 60, JGI20 sequence is SEQ ID NO: 61. (b) Screening result of the structure hybrid of the two proteins by single residue insertion/deletion represented by glycolate productivity (μM/OD/h) with respect to the wildtype JGI20 using formaldehyde (0.5 or 5 mM) and formate (20 mM) system with AbfT co-expression;

FIG. 27 depicts (a) Sequence and structure of the c-terminal tail “covering loop” between JGI15 (SEQ ID NO: 25) and JGI20 (SEQ ID NO: 26) (b) Screening result of the structure hybrid by replacing JGI20 c-terminal end with JGI15 represented by glycolate productivity (μM/OD/h) with respect to the wildtype JGI20 using formaldehyde (0.5 or 5 mM) and formate (20 mM) system with AbfT co-expression;

FIG. 28 depicts (a) Glycolate production from formaldehyde via 2-hydroxyacyl-CoA synthase (HACS) and acyl-CoA reductase (LmACR) activity. 5 mM formaldehyde is used as the sole carbon source. (b) Two inducible promoter, HACS under control of IPTG-inducible T7 promoter and LmACR and EcAldA under control of cumate-inducible T5 promoter are used for analysis. (c) Glycolate productivity of RuHACL, three best first round mutants and AcHACL under 5 mM formaldehyde;

FIG. 29 Identification of 2^{nd} round JGI HACS homologs based on AcHACL, JGI19, JGI15 and JG120 as the starting reference. The phylogenetic tree includes all first round and second round HACS variants as well as HACLs and OXCs available in literature;

FIG. 30 depicts (a) Glycolate production from co-feeding formaldehyde (0.5 mM) and formate (20 mM). Expression of HACS and FAE (AbfT) are controlled independently by inducible promoters (IPTG and cumate) (b) HACS screening results of the promising 2^{nd} round variants represented by % change in glycolate productivity with respect to JGI15 as a reference;

FIG. 31 depicts (a) 2-Hydroxyacids production from co-feeding aldehydes and formate (b) Expression of HACS and FAE (CaAbfT) are controlled independently by inducible promoters (IPTG and cumate);

FIG. 32 depicts (a) Lactic acid (lactate) production from co-feeding acetaldehyde (5 mM) and formate (20 mM) (b) HACS screening results of the promising 1^{st} round variants represented by lactate productivity (uM/OD) (c) HACS screening results of the promising 2^{nd} round variants represented by % change in lactate productivity with respect to JGI15 as a reference;

FIG. 33 depicts (a) 2-Hydroxybutyric acid (2HB) production from co-feeding propionaldehyde (5 mM) and formate (20 mM) (b) HACS screening results of the promising 1^{st} round variants represented by 2HB productivity (μM/OD) (c) HACS screening results of the promising 2^{nd} round variants represented by % change in 2HB productivity with respect to JGI15 as a reference;

FIG. 34 depicts (a) Glyceric acid (glycerate) production from co-feeding glycolaldehyde (5 mM) and formate (20 mM) (b) HACS screening results of the promising 1^{st} round variants represented by glycerate productivity (μM/OD);

FIG. 35 depicts (a) Tartronic acid (tartronate) production from co-feeding glyoxylate (5 mM) and formate (20 mM) (b) HACS screening results of the promising 1^{st} round variants represented by tartronate productivity (μM/OD);

FIG. 36 depicts (a) 2,4-Dihydroxybutyric acid (DHB) production from co-feeding 3-hydroxypropionaldehyde (5 mM) and formate (20 mM) (b) HACS screening results of the promising 1^{st} round variants represented by DHB productivity (μM/OD);

FIG. 37 depicts Screening of first round HACS with acetone and formate. (a) 2HIB production from co-feeding 100 mM acetone and 20 mM formate. Expression of HACS and FAE (CaAbfT) are controlled independently by inducible promoters (IPTG and cumate), (b) HACS screening results of the promising HACS variants;

FIG. 38 depicts Methyl ketones as substrate for condensation with formyl-CoA using purified enzymes. (a) Pathway for condensation of methyl ketones and formyl-CoA from formate to 2-hydroxy-2-methyl acid; (b) GC-MS results of in vitro assays with different methyl ketones and formate. The desired product was pointed with blue arrow;

FIG. 39 depicts production of 2-hydroxyacid, 3-hydroxyacid, alcohol, 1,2-diol and α,β-unsaturated acid from condensation of carboxylic acid-derived ketones and formyl-CoA;

FIG. 40 depicts production of 2-hydroxyisobutyric acid, 3-hydroxyisobutyric acid, isobutanol, isobutene glycol and methacrylic acid from condensation of lactic acid-derived acetone and formyl-CoA;

FIG. 41 depicts production of 2-hydroxy-2-methylbutanoic acid, 3-hydroxy-2-methylbutanoic acid, 2-methylbutan-1-ol, 2-methylbutane-1,2-diol and 2-methylbut-2-enoic acid from condensation of 2-hydroxybutanoic acid-derived butanone and formyl-CoA;

FIG. 42 depicts production of 2-hydroxy-2-methylpentanoic acid, 3-hydroxy-2-methylpentanoic acid, 2-methylpentan-1-ol, 2-methylpentane-1,2-diol and 2-methylpen-2-enoic acid from condensation of 2-hydroxypentanoic acid-derived pentanone and formyl-CoA;

FIG. 43 depicts production of 2-hydroxy-2-methylheptanoic acid, 3-hydroxy-2-methylheptanoic acid, 2-methylheptan-1-ol, 2-methylheptane-1,2-diol and 2-methylhept-2-enoic acid from condensation of 2-hydroxyheptanoic acid-derived heptanone and formyl-CoA;

FIG. 44 depicts production of 2,3-hydroxy-2-methylproptanoic acid, 2-methylpropane-1,3-diol, 2-methylpropane-1,2,3-triol and 3-hydroxy-2-methylacrylic acid from condensation of 2,3-hydroxypropanoic acid-derived hydroxyacetone and formyl-CoA;

FIG. 45 depicts production of 2-hydroxy-2,3-dimethylbutanoic acid, 3-hydroxy-2,3-dimethylbutanoic acid, 2,3-dimethylbutan-1-ol, 2,3-dimethylbutane-1,2-diol and 2,3-dimethylbut-2-enoic acid from condensation of 2-hydroxy-3methylbutanoic acid-derived 3-methyl-2-butanone and formyl-CoA;

FIG. 46 depicts production of 2-hydroxy-2-methyl-3-oxopropanoic acid, 2-methylpropane-1,3-diol, and 2-methylheptane-1,2,3-triol from condensation of 2-hydroxy-3-oxopropanoic acid-derived methylglyoxal and formyl-CoA;

FIG. 47 depicts production of 2-hydroxy-2-methyl-4-oxopentanoic acid, 3-hydroxy-2-methyl-4-oxopentanoic acid, 5-hydroxy-4-methylpentan-2-one, 4,5-dihydroxy-4-methylpentan-2-one and 2-methyl-4-oxopent-2-enoic acid from condensation of 2-hydroxy-4-oxopentanoic acid-derived pentane-2,4-dione and formyl-CoA;

FIG. 48 depicts (a) Glycolate production from formaldehyde via 2-hydroxyacyl-CoA synthase (RuHACL) and acyl-CoA reductase (ACR) variants. (b) Screening results of initial ACR variants identified from literature;

FIG. 49 depicts (a) Phylogenetic tree diagram of the ACR variants identified using LmACR as the starting reference. (b) Screening of ACR variants was done by measuring formaldehyde consumption under ACR variants overexpression. (c) Screening result of ACR variants under 0.5 mM and 3 mM formaldehyde concentrations represented by % change in formaldehyde consumption activity with respect to LmACR as a reference;

FIG. 50 depicts (a) Glycolate production from formaldehyde and formate via 2-hydroxyacyl-CoA synthase (JGI15) and formate activation enzyme variants. PTA-ACK generates formyl-CoA via formyl-phosphate intermediate while ACT transfers CoA directly to formate from CoA donor molecule (b) Screening results of initial ACT & PTA-ACK variants identified from literature;

FIG. 51 depicts (a) Phylogenetic tree diagram of the ACT variants identified using AbfT as starting reference. (b) Phylogenetic tree diagram of the ACT variants identified using CcAck-Pta as starting reference;

FIG. 52 depicts (a) Glycolate production from formaldehyde and formate via 2-hydroxyacyl-CoA synthase and formate activation enzyme variants. PTA-ACK generates formyl-CoA via formyl-phosphate intermediate while ACT transfers CoA directly to formate from CoA donor molecule. (b) Screening results of the promising ACT and ACK-PTA variants for glycolate productivity (μM/OD/h);

FIG. 53 depicts (a) Engineering of glycolate auxotroph strain by enforcing glycolate to be the sole source for glycine synthesis. This strain can only grow either when glycine is supplemented or glycolate with appropriate enzymes to synthesize glycine is provided. (b) The engineered glycolate auxotroph strain is able to grow only with glycolate supplementation showing higher growth rate with increasing glycolate concentrations.

### DETAILED DESCRIPTION

The term “about,” as used herein, refers to variations in the numerical quantity that may occur, for example, through typical measuring and manufacturing procedures used for articles of footwear or other articles of manufacture that may include embodiments of the disclosure herein; through inadvertent error in these procedures; through differences in the manufacture, source, or purity of the ingredients used to make the compositions or mixtures or carry out the methods; and the like. Throughout the disclosure, the terms “about” and “approximately” refer to a range of values ±5% of the numeric value that the term precedes.

In the canonical ‘bow-tie’ architecture of metabolism substrates are funneled into central metabolism with biosynthetic building blocks and products of interest derived from the resulting central metabolites. To date, attempts to engineer C1 bioconversion, have required central carbon metabolism for the utilization of C1 substrates and their conversion to products of interest. These designs, which exhibit minimal orthogonality, have required optimizing a host's metabolic network to accommodate C1 bioconversion, which has proven challenging.

However, implementation of formyl-CoA elongation (FORCE) pathways, enabling C1 utilization and bioconversion in a manner orthogonal to the host metabolism, may resolve these challenges. FORCE pathways are based on the use of formyl-CoA as an anabolic metabolite, which is enabled by acyloin condensation reactions between formyl-CoA and carbonyl-containing substrates catalyzed by 2-hydroxyacyl-CoA lyase (HACL). Product synthesis is achieved with relatively high orthogonality to central metabolism compared to other approaches. Our analysis of pathway thermodynamics suggested favorable driving forces for FORCE pathway conversions of formate, formaldehyde, and methanol to glycolate or acetate as exemplary products. Self-contained, orthogonal pathways are shown to be potentially viable in both in vitro (purified enzymes and cell extracts) and in vivo (resting and growing cells) implementations, in which products of diverse functionality (e.g. glycolate, glycolaldehyde, ethylene glycol, ethanol, glycerate) could be produced in a growth and host metabolism independent manner using formaldehyde, formate, or methanol as the sole C1 substrates. Product synthesis demonstrated here completely bypasses central metabolism, which is distinct from all other approaches reported to date. One can envision potential bioprocesses in which growth and maintenance of the biocatalyst is performed with a multi-carbon substrate, and the biocatalyst is used for C1 bioconversions. Bioprocesses of this nature, based on multi-enzyme cascades and two-phase fermentations, have been the subject of recent reviews.

### Design of an Orthogonal Metabolic Architecture for C1 Utilization and Product Synthesis

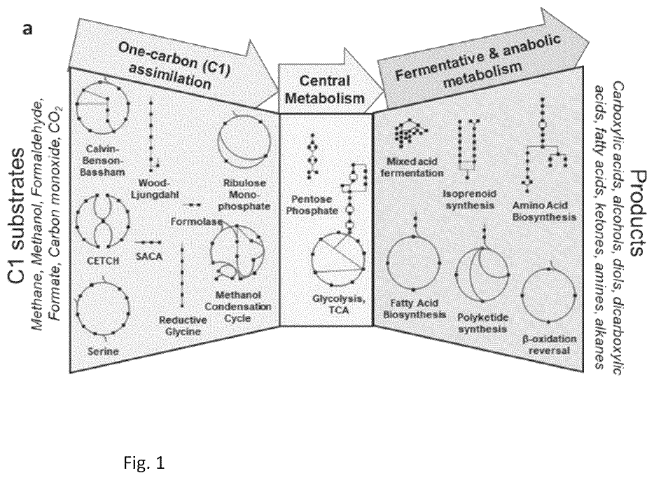

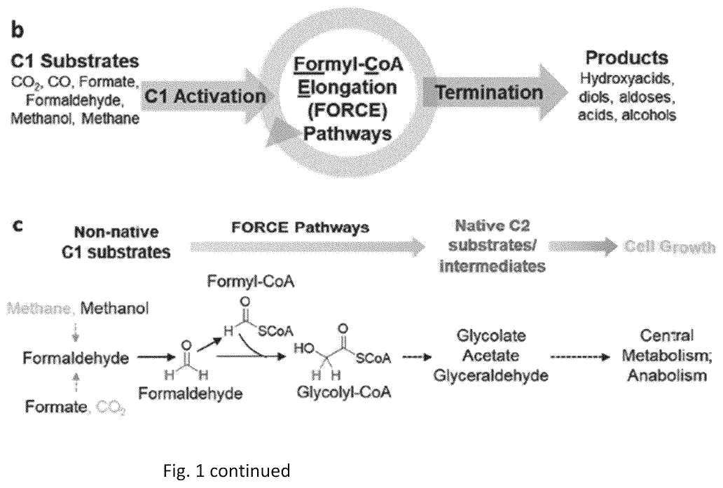

Some embodiment FORCE pathways that can provide bioconversion of C1 substrates into desirable products are discussed below and shown in the figures. Referring specifically to the example shown in in FIGS. 1a and 1b, some embodiment systems may have three primary features of the orthogonal metabolic architecture: 1) activation of C1 substrates into a suitable building block for carbon chain elongation; 2) iterative elongation of a carbon chain by one carbon per cycle; and 3) termination of the pathway resulting in accumulation of the product of interest. For example, in an embodiment, and orthogonal metabolic architecture having these features could be implemented using formyl-CoA as the activated C1 unit for iterative carbon chain elongation.

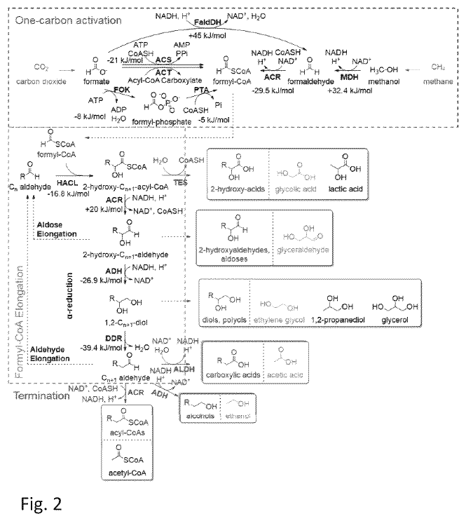

In existing literature, reports of the generation of formyl-CoA from C1 molecules are sparse. Acyl-CoAs, though, are a convenient intermediate between the carboxylate and aldehyde forms. As a result, as shown in the one-carbon activation panel of FIG. 2, it is possible to produce formyl-CoA from both oxidized and reduced C1 substrates. From formaldehyde, formyl-CoA can be produced by the activity of acyl-CoA reductase (ACR), and methanol can be converted to formaldehyde by methanol dehydrogenase (MDH).

Formyl-CoA may be produced from formate by the use of CoA transferases. Formyl-CoA transferase is one such enzyme known to involve formate and formyl-CoA in CoA thioester transfer. Activation of formate to formyl-CoA by the promiscuous activity of acetyl-CoA synthetase (ACS) from Escherichia coli (EcACS) is all possible. While the reaction catalyzed by EcACS is AMP forming (consuming 2 ATP equivalents), evidence of an ADP forming route exists via the intermediate formyl-phosphate. In this route, formate is converted to formyl-phosphate by formate kinase (FOK) and phosphotransacylase (PTA) converts formyl-phosphate to formyl-CoA. It may also be possible to convert formate to formyl-CoA in an ATP-independent manner via the direct reduction of formate to formaldehyde by formaldehyde dehydrogenase (FaldDH). Although such a conversion would be thermodynamically challenging, as demonstrated in FIG. 2, this reaction has been demonstrated in cell-free systems and could potentially be useful for both in vitro and in vivo implementations. In addition, CO₂ can be converted to formate by the reverse activity of formate dehydrogenase (or carbon dioxide reductase) and methane to methanol by methane monooxygenase, which when coupled to the reactions described above can lead to the formation of formyl-CoA.

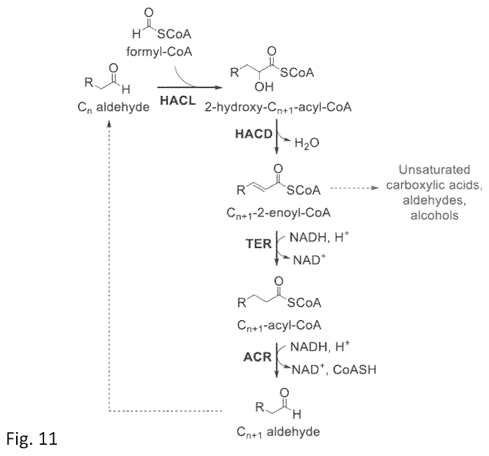

An orthogonal, de novo construction of diverse carbon skeletons by elongation using C1 units necessitates an iterative pathway similar to those found in nature that construct carbon skeletons from C2-C5 metabolites, yet existing outside of central metabolism. Because 2-hydroxyacyl-CoA lyase (HACL) has broad carbon chain length specificity, it is a good candidate for establishing an iterative pathway. There exist numerous potential reaction pathways that might enable iteration by converting the product of the HACL-catalyzed reaction, 2-hydroxyacyl-CoA, to an aldehyde that can be further extended by formyl-CoA. As shown in FIG. 11, at the α-carbon, dehydration is possible, transforming the 2-hydroxyacyl-CoA to a 2-enoyl-CoA FIG. 11 similar to the mechanism of the well-established acrylate pathway. The production of 2-enoyl-CoA is also convenient because 2-enoyl-CoAs are involved in β-oxidation, potentially allowing the use of the enzymatic toolkit and knowledge established for the platform pathway β-oxidation reversal. Dehydration of 2-hydroxyacyl-CoA, however, is much more challenging than dehydration of 3-hydroxyacyl-CoA in β-oxidation reversal, thus requiring an oxygen-sensitive radical mechanism. Dehydration of the 2-hydroxyacyl-CoA also requires the existence of a β-carbon and thus limits the implementation of the pathway to intermediates 3 carbons or larger.

These issues make transformations of the thioester a more promising pathway. As shown in FIG. 2, and the formyl-CoA elongation panel, reduction of the CoA-thioester would give a 2-hydroxyaldehyde, which is possible due to the non-specific activity of certain acyl-CoA reductases (ACRs). Further ligation of 2-hydroxyaldehydes with formyl-CoA by HACL give polyhydroxyacyl-CoAs and further polyhydroxyaldehydes, commonly known as aldoses. Polyhydroxyaldehydes can in principle serve as substrates of the HACL-catalyzed reaction, which can be referred to as aldose elongation, and an example of this is shown in FIG. 2, at the formyl-CoA elongation panel).

Further reduction of the 2-hydroxyaldehyde to give a 1,2-diol is possible by the activity of a diol oxidoreductase (DOR). E. coli FucO is an example of a DOR which catalyzes the interconversion of 1,2-diols with 2-hydroxyaldehydes34. However, E coli is only one example of a DOR, and in some examples other suitable DORs may instead be used. For example, in some embodiments, the DOR may be another prokaryotic bacteria. Alternatively, in some embodiments, the DOR may be a eukaryotic bacteria or a fungi. Dehydration of 1,2-diol can be catalyzed by the activity of diol dehydratase (DDR) to give an aldehyde, effectively accomplishing α-reduction. While diol dehydration also requires a radical mechanism, the B12-dependent diol dehydratase is oxygen tolerant. Further elongation of the aldehyde by formyl-CoA, which can be referred to as aldehyde elongation, results in the extension of an alkyl chain, analogous to the two-carbon elongation in fatty acid biosynthesis or reverse β-oxidation pathways. These pathways, which comprise aldose elongation, can be collectively referred to as α-reduction, and aldehyde elongation, as formyl-CoA elongation (FORCE) pathways, as they facilitate the use of formyl-CoA as a carbon chain elongation unit, as shown in FIG. 2, at hteformyl-CoA elongation panel.

As shown in FIG. 2, a variety of product classes can be produced as intermediates or from derivatives of intermediates of FORCE pathways, some of which also can support microbial growth (shown in FIG. 1c). Aldose sugars, for example, are a direct result of the 2-hydroxyaldehyde node. Diols, including major industrial chemicals such as ethylene glycol, are a result of the 1,2-diol node. Derivatives of the 2-hydroxyacyl-CoA node include 2-hydroxyacids, such as industrial products glycolic and lactic acids, produced by a reaction catalyzed by thioesterases. Numerous chemical classes can be derived from the aldehyde node, including carboxylic acids, alcohols, and acyl-CoAs that can serve as precursors of other products.

### Thermodynamic Analysis of FORCE Pathways for C1 Utilization

The standard Gibbs free energies of the pathway reactions shown in FIG. 2 makes readily apparent the potential reactions, but the Gibbs energies are insufficient to predict whether the pathway as whole will be feasible and operate in the intended direction. For this, a holistic approach to the pathway thermodynamics is necessary that considers the ability for pathway reactions to influence each other. To accomplish this, a “Max-min Driving Force (MDF)” approach was applied to these reactions.

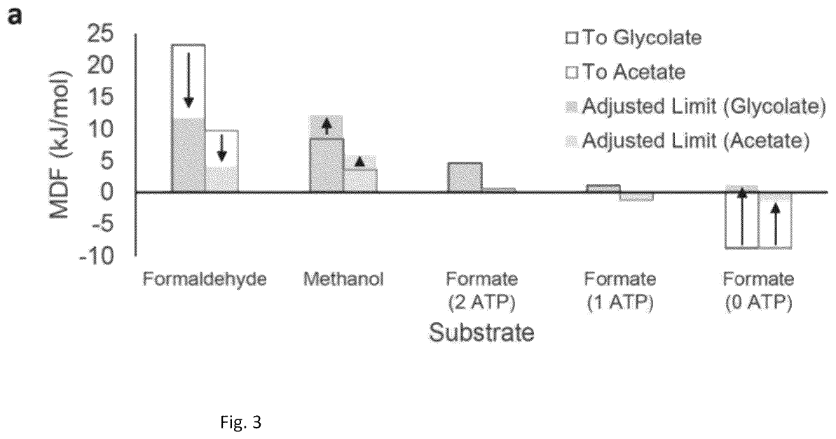

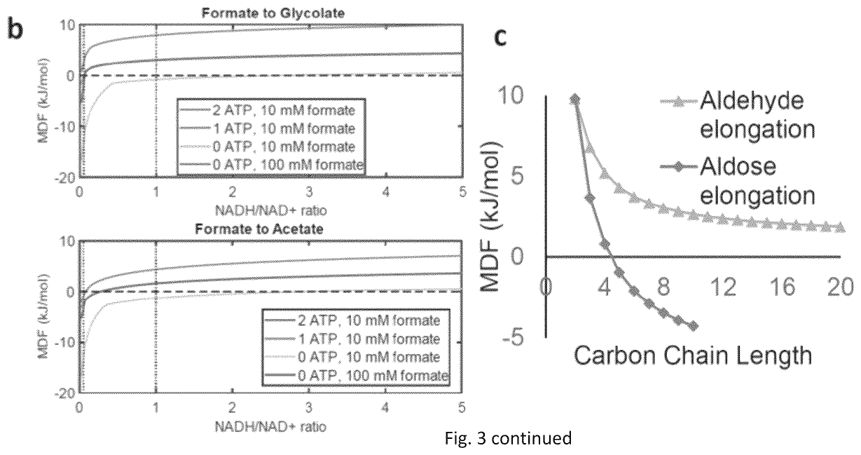

The MDF of the FORCE pathways for the production of C2 metabolites glycolate and acetate from solely C1 substrates was evaluated, however, only the MDFs of soluble C1 substrates were evaluated, as mass transport limitations are likely to significantly limit CO₂ and methane utilization. Glycolate and acetate were chosen as representative C2 products that are both pathway products and growth substrates, with glycolate requiring the shortest pathway and acetate requiring the entire sequence of aldehyde elongation reactions. As shown in FIG. 3a, there is a greater driving force toward the production of glycolate than acetate for each substrate. This is due to the thermodynamically favorable hydrolysis of glycolyl-CoA by thioesterase, whereas the production of acetate requires the thermodynamically challenging reduction of glycolyl-CoA. Using standard limits of metabolite concentrations, formaldehyde allows the greatest MDF as it does not require the challenging NAD+-dependent methanol dehydrogenase reaction or formyl-CoA reduction reaction to formaldehyde. It is also evident that despite the thermodynamic challenge of the methanol dehydrogenase reaction, there is sufficient driving force in the preferred direction for the net production of glycolate and acetate.

The driving force for formate utilization is the lowest. Here, ATP hydrolysis assists in the activation of formate. The hydrolysis of 2 ATP equivalents by ACS provides just enough driving force for the net production of acetate, while the utilization of 1 ATP equivalent only provides enough driving force for the production of glycolate. The ATP-independent route is not feasible under these conditions.

While the above analysis assumes a standard constraint on metabolite concentrations from 1 μM to 10 mM, in practice the C1 substrate concentration can be higher or lower than this upper bound based on the ability to exogenously supply it. Next, as shown in FIG. 3, more stringent/realistic values were used for substrate concentrations based on physical limitations such as the toxicities of the C1 substrates. Although some organisms can survive and consume formaldehyde concentrations on the order of 10 mM42, most organisms, including the bacterium

E. coli cannot. When the upper bound of formaldehyde was adjusted to a more reasonable 0.1 mM, the MDF of the pathways decreased as expected. Methanol, on the other hand, is much less toxic than formaldehyde and has been supplied to E. coli growth media at concentrations on the order of 100 mM43. Increasing the upper bound on methanol concentration increased the MDF of methanol conversion. Interestingly, at these concentrations, the driving force for methanol utilization becomes slightly greater than that for formaldehyde.

Similarly, E. coli has the ability to grow in the presence of formate concentrations on the order of 100 mM9. In other embodiments, other DORs that grow in the presence of formate concentrations could also be used. Increasing the bound on formate concentration had no effect on the MDF in the 1 or 2 ATP consumption scenarios, but it had a major impact on the MDF of the 0 ATP route. With 100 mM formate, net production of glycolate, but not acetate, is possible without the need for ATP hydrolysis. This analysis can inform cell-free bioconversion systems and provide valuable insights with regards to substrate uptake for in vivo implementations.

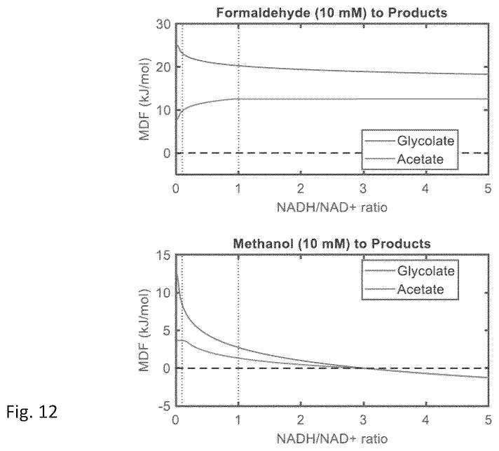

Aside from the substrate concentration, the NADH/NAD+ ratio is the other major constraint to the pathway thermodynamics. While the previously used constraint on NADH/NAD+ was 0.141, reflecting growth of E. coli under aerobic conditions, the physiological NADH/NAD+ can vary, reaching values near or greater than 1 under anaerobic conditions. Even higher ratios can be achieved in in vitro implementations. To assess the influence of NADH/NAD+ ratio on the pathway driving force, the NADH/NAD+ ratio was varied. As shown in FIG. 12, in the physiological range (taken here to be between 0.1-1), pathway driving forces remained positive for formaldehyde and methanol as substrates FIG. 12. As expected, a low ratio was favorable when the pathway was redox generating (methanol to acetate/glycolate and formaldehyde to glycolate), while a high ratio was favorable when the pathway was redox consuming (formate to acetate/glycolate) or redox balanced (formaldehyde to acetate—likely because the reduction reactions are more thermodynamically challenging). As shown in FIG. 3b, the NADH/NAD+ ratio can be critical for the driving force of the formate utilization pathways.

For the conversion of formate to glycolate, the route requiring 1-2 ATP equivalents retains a positive driving force throughout nearly the entire physiological range. For the conversion of formate to acetate, though, the NADH/NAD+ ratio must be on the higher end of the physiological range to have a positive driving force with the consumption of 1 ATP equivalent. At 10 mM formate, neither the driving force for glycolate nor acetate production is positive in the physiological range. When the concentration of formate is increased to 100 mM, the driving force for glycolate or acetate production can be positive within the physiological range of NADH/NAD+ ratios even without ATP hydrolysis. Overall, the conversion of formate to more reduced products such as acetate is challenged both thermodynamically and on the basis of net redox balance.

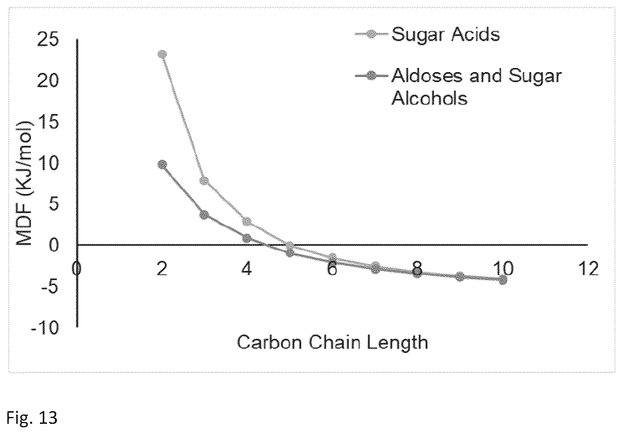

As one example of an embodiment method of converting C1 substrates into products, the ability of the FORCE pathways to support iteration using formaldehyde as the exemplary substrate due to its intermediate redox state was further evaluated. As shown in FIG. 3c, the thermodynamics of both the aldose and aldehyde elongation pathways support iteration up to 4 carbons. In addition, the driving force of the pathway decreases with the number of iterations. After 4 carbons, the aldose elongation mode becomes unfavorable, likely due to the cumulative effect of successive acyl-CoA reduction reactions. The aldehyde elongation mode, on the other hand, remains favorable despite also requiring the same acyl-CoA reductions, likely due to the thermodynamically favorable reactions catalyzed by DOR and DDR. Different C1 activation and termination pathways have an influence on the MDF of the overall elongation cycles when the number of iterations is low. As shown in FIG. 13, as the number of iterations increases, the thermodynamics of the elongation cycle reactions dominate FIG. 13. Based on the preceding analysis, it can be expected that the MDF of the aldose and aldehyde elongation pathways will be similar or lower when methanol or formate are the C1 substrate because utilization of these substrates is more thermodynamically constrained.

### In Vitro Pathway Validation

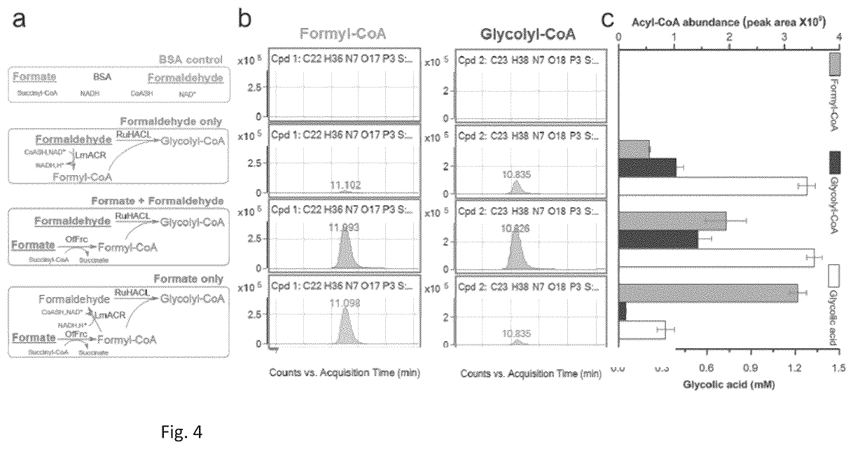

A prerequisite to the FORCE pathways is the generation of formyl-CoA and formaldehyde. To verify the function of these reactions, as shown FIG. 4, purified enzyme systems were developed and the formation of both formyl-CoA and of the HACL condensation product glycolyl-CoA from different C1 substrates was tracked. As indicated in FIG. 2, formyl-CoA can be produced from formaldehyde by an acyl-CoA reductase (ACR). In a reaction containing both Listeria monocytogenes ACR (LmACR) and HACL from Rhodospirillales bacterium URHD0017 (RuHACL), the formation of both formyl-CoA and glycolyl-CoA was observed. Formyl-CoA can also be derived from oxidized C1 substrates by the activation of formate. Using a formyl-CoA transferase from Oxalobacter formigenes (OfFrc) and succinyl-CoA as the CoA donor, the activation of formate to formyl-CoA was observed, and, with the addition of formaldehyde, resulted in the formation of glycolyl-CoA. Whether formaldehyde could be produced in situ by the reduction of formyl-CoA using LmACR was further tested. Indeed, glycolyl-CoA was observed, although its abundance was less than when formaldehyde was added directly. Taken together, these results suggest that in this enzyme system, the limitation is imposed by the ACR reaction either due to the activity of the enzyme or the constraints placed due to the need for the appropriate form of NAD (H). In support of the latter, in the oxidative direction (i.e. formaldehyde to formyl-CoA), the amount of glycolate observed following hydrolysis of the CoA thioesters was nearly equivalent to the amount of NADH added to the reaction (1 mM). A shown in FIGS. 2 and 3b, in the opposite direction, a less than equivalent amount of glycolate was observed, which is consistent with the thermodynamics of the reaction becoming unfavorable as the ratio of NADH/NAD+ decreases.

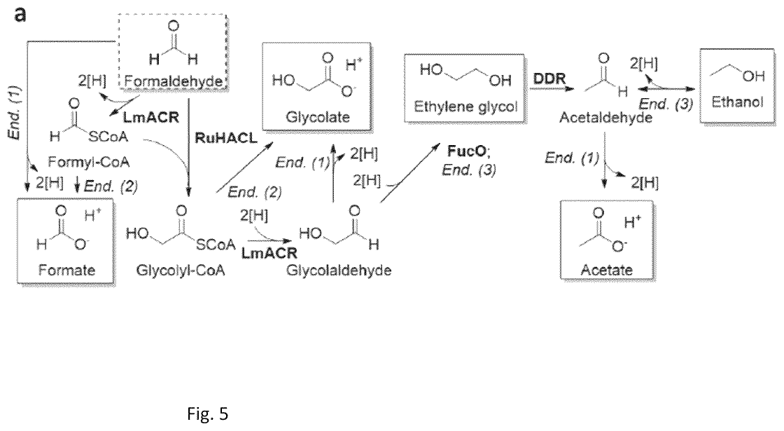

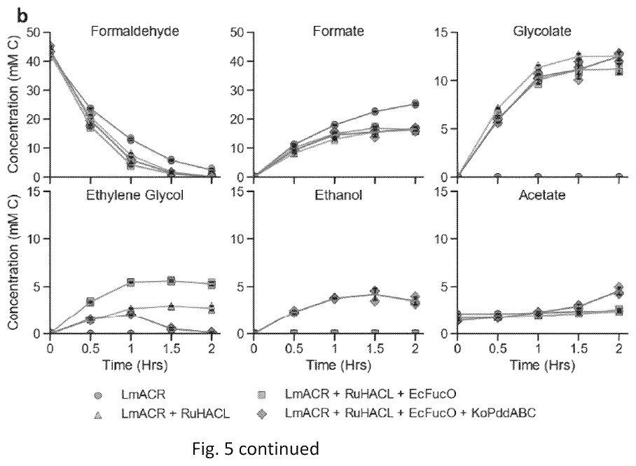

A cell-free metabolic engineering approach was used to further prototype the FORCE pathways for product synthesis. Extracts of E. coli expressing each pathway enzyme comprising the α-reductive FORCE pathway were successively combined, demonstrating the pathway functions in a stepwise manner. As shown in FIG. 2 (in the formyl-CoA elongation panel), outside of the direct generation of 2-hydroxycarboxylates (e.g. glycolate) via thioester cleavage of the 2-hydroxyacyl-CoA generated by HACL, other C2 products, (including the aldose or aldehyde elongation pathways) require reduction of this 2-hydroxyacyl-CoA (glycolyl-CoA in the case of formaldehyde and formyl-CoA ligation). Listeria monocytogenes ACR (LmACR) are also able to act upon glycolaldehyde. As shown in FIG. 5a, to minimize the complexity of the engineered system, LmACR was used in a bifunctional role, catalyzing both the oxidation of formaldehyde to formyl-CoA and the reduction of glycolyl-CoA to glycolaldehyde. As shown in FIG. 5b, LmACR alone resulted in only the conversion of formaldehyde to formate. With the inclusion of the previously identified HACL from Rhodospirillales bacterium URHD0017 (RuHACL), glycolate was observed. Glycolaldehyde, however, was not significantly detected as a product, possibly due to the presence of endogenous oxidoreductases in the cell extract system, which catalyzed the oxidation of glycolaldehyde to glycolate (e.g. AldA, AldB, PuuC, PatD) or, to a lesser extent, reduction to ethylene glycol (e.g. FucO, YqhD, AdhP, EutG, and others).

As shown in FIGS. 5a and 5b, the synthesis of the next reduction product, ethylene glycol, was significantly increased by the addition of a cell extract of E. coli overexpressing E. coli FucO, a 1,2-diol oxidoreductase (a 2-fold increase, from 1.37±0.1 mM to 2.73±0.03 mM). Ethylene glycol can be further dehydrated to acetaldehyde by a diol dehydratase (shown in FIG. 5a). Upon further addition of E. coli cell extract expressing diol dehydratase (DDR) from Klebsiella oxytoca, along with coenzyme B12, ethanol was detected (1.90±0.03 mM at one hour: FIG. 5b), likely due to the reduction of acetaldehyde by endogenous oxidoreductases, along with a corresponding decrease in ethylene glycol. At later time points (2 hours), an increase in acetate was observed, likely due to the oxidation of ethanol and acetaldehyde again due to the presence of endogenous oxidoreductase activity. Synthesis of the varied products (i.e. glycolate, ethylene glycol, ethanol, glycerate, acetate) illustrate that judicious selection of pathway enzymes can be used to control the synthesis of products of varying levels of reduction, chain lengths, and functionalities from the acyl-CoA node, all independent of central metabolism.

### In Vivo Implementation of FORCE Pathways

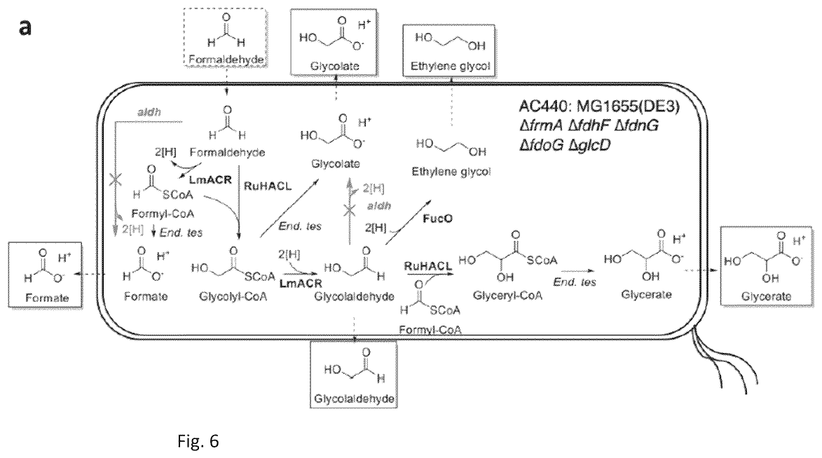

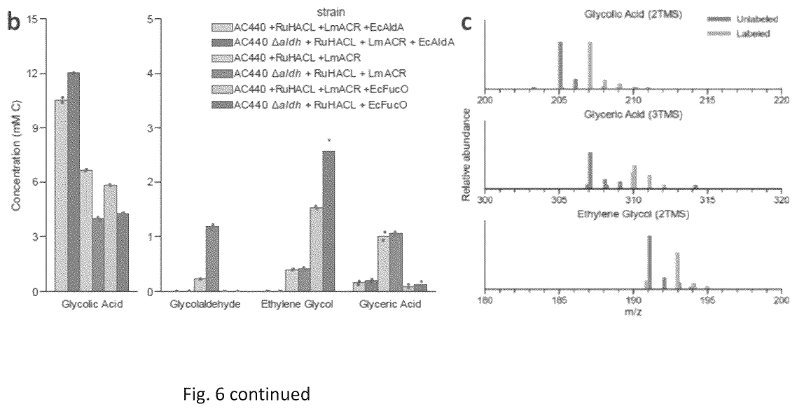

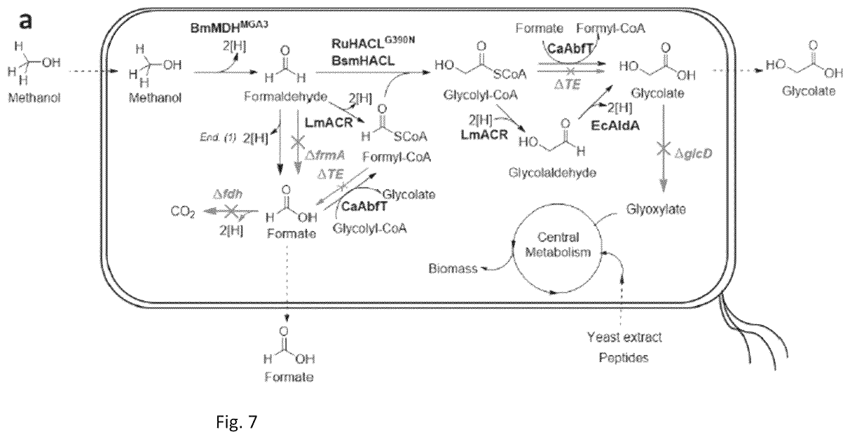

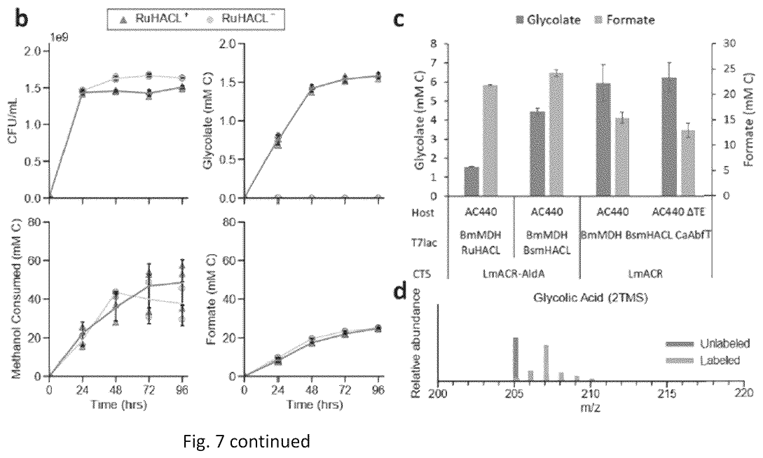

The orthogonal nature of FORCE pathways allows not only for the rapid prototyping in cell-free systems, but also the facile in vivo implementation. FIGS. 6 and 7 demonstrate the key features of the designed platforms, as well as the synthesis of additional products and utilization of various C1 substrates using both resting and growing cultures of E. coli. A key feature of the FORCE pathway design is iteration, which can be achieved through aldose or aldehyde elongation (as shown in the formyl-CoA elongation panel of FIG. 2 and in FIG. 3c). To demonstrate the feasibility of iterative aldose elongation in vivo, the synthesis of three carbon product glycerate from formaldehyde was targeted, as shown in FIG. 6a. A strain having C1 dissimilation and glycolate consumption knockouts (AC440: MG1655(DE3) ΔfrmA ΔfdhF ΔfdnG ΔfdoG ΔglcD) and (over) expressing RuHACLG390N, LmACR, and EcAldA16 was used. To promote the accumulation of glycolaldehyde and its condensation with formyl-CoA, EcAldA was removed from the expression vector. While the consumption of formaldehyde was significantly reduced, FIG. 6b shows that accumulation of glycolaldehyde and glycerate was observed, demonstrating the iterative aldose elongation pathway. In an attempt to increase the production of these compounds, the genes encoding aldehyde dehydrogenases (ΔaldA ΔaldB ΔpatD ΔpuuC, collectively referred to as Δaldh) were further knocked out. Using this host, the concentration of the oxidation product, glycolate, decreased, while glycolaldehyde concentration increased when EcAldA was not overexpressed. Despite this, the knockouts did not appear to impact the accumulation of glycerate, perhaps indicating a limitation on the condensation reaction between glycolaldehyde and formyl-CoA, which is catalyzed by RuHACL. The knockouts also did not have an impact on the accumulation of the byproduct formate, indicating that the likely route of byproduct formation is via thioester hydrolysis of formyl-CoA.

The pathway was also extended beyond the production of glycolaldehyde to the next reduction product, ethylene glycol, by including E. coli fucO in the expression vector, which is known to catalyze the interconversion of glycolaldehyde and ethylene glycol. As shown in FIG. 6b, this led to the accumulation of ethylene glycol in the extracellular medium. The additional knockout of aldehyde dehydrogenases resulted in an approximately 68% increase in ethylene glycol production. Interestingly, inclusion of EcFucO dramatically increased the consumption of formaldehyde, with much of it converted to formate. This might be explained by the net redox balance for ethylene glycol production from formaldehyde, which requires an additional reducing equivalent in the form of NADH. This would decrease the NADH/NAD+ ratio and provide additional electron acceptors for formaldehyde oxidation.

To verify that the observed products were derived from formaldehyde and not from residual multi-carbon substrates or biomass components, 13C-labeled formaldehyde was used as the substrate for the engineered strains. As shown in FIG. 6c, the products glycolic acid, ethylene glycol, and glyceric acid were found to be fully 13C labeled based on the characteristic [M-15]+ ions of the TMS derivatives of the products. Both 2 and 3 carbon products could therefore be produced from formaldehyde using the FORCE pathways.

In addition to varied products, whether different substrates could be utilized was also assessed. As shown in FIGS. 2, 7a, only the additional expression of a methanol dehydrogenase (MDH) is necessary to convert methanol to formaldehyde, which is a convenient extension of the previously established formaldehyde utilization pathway. A well-studied MDH variant from Bacillus methanolicus MGA3 (BmMDH2MGA3) was expressed in combination with RuHACLG390N, LmACR, and EcAldA in strain AC440. Unlike formaldehyde, where toxicity necessitates the use of resting cells, methanol can also be directly added to growing E. coli cultures. As shown in FIG. 7b, when the engineered methanol utilizing strain was grown in the presence of complex nutrients and 500 mM methanol, the formation of glycolate was observed, which was not the case in a strain not expressing RuHACL.

Seeking to improve upon the performance of this system, RuHACLG390N was replaced with a newly identified HACL sourced from beach sand metagenome referred to here as BsmHACL (UniProt accension: A0A3C0TX30). As shown in FIG. 7c, the use of BsmHACL substantially increased glycolate accumulation about 3-fold, reflecting a major bottleneck in the pathway. Despite improved glycolate production, formate accumulation remained high. In an effort to address this issue, the termination enzyme EcAldA was replaced with a previously identified CoA-transferase from Clostridium aminobutyricum (CaAbfT), which was found to have better properties than OfFrc51. CaAbfT serves to both release glycolate from glycolyl-CoA and to reactivate the observed byproduct formate to formyl-CoA for further condensation. When CaAbfT was expressed, glycolate accumulation further increased by around 33%, while formate accumulation was reduced by around 36%. Finally, with CaAbfT serving as a way to terminate the pathway via the release of glycolate, endogenous thioesterases were not expected to be needed and were presumed to be responsible at least in part for the observed formate. A strain deficient in thioesterases (ΔyciA ΔtesA ΔtesB ΔybgC ΔydiI ΔfadM) was therefore constructed and tested with the pathway. Using the thioesterase deficient background further reduced the observed formate but did not eliminate it. It is possible that there are other routes of formate production such as the direct oxidation of formaldehyde or not yet identified thioesterases. To verify that the observed glycolate was derived from methanol, 13C-labeled methanol was further employed. As shown in FIG. 7d, as observed from the [M-15]+ ion of the TMS derivative of glycolic acid, the glycolate produced in these cultures was fully derived from the 13C-methanol.

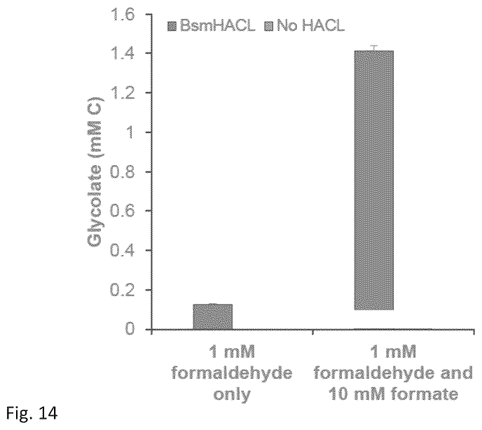

Having established CaAbfT as a promising route for formate activation, whether CaAbfT could be used to enable the incorporation of exogenously supplied formate was further evaluated. In an engineered strain of E. coli, CaAbfT was expressed to activate formate, while LmACR was not expressed such that there was no interconversion of formaldehyde and formyl-CoA. Therefore, the observed glycolate should result from formate activation to formyl-CoA and further condensation of the resulting formyl-CoA with formaldehyde. As shown in FIG. 14, in the engineered strain expressing BsmHACL, a 12-fold increase in glycolate was observed when formate was included in the media in addition to formaldehyde compared to when formaldehyde was supplied alone. FIG. 14, and the total carbon accumulated as glycolate was greater than the amount originally added as formaldehyde. Thus, the production of glycolate by this strain was formate-dependent, which serves as a demonstration that exogenously supplied formate can be used as a C1 substrate with FORCE pathways.

### Flux Balance Analysis of FORCE Pathways for Synthetic Methylotrophy

Having demonstrated the potential for the FORCE pathways to support product synthesis and because some of the products (e.g. glycolate, glycerate, acetate) can serve as growth substrates, their ability to enable synthetic methylotrophy in E. coli was evaluated in silico. Using a genome scale model of E. coli, iML151552, growth of E. coli on organic C1 substrates was evaluated by the addition of reactions to the model comprising select pathways reported or proposed to enable methylotrophy. All pathways were evaluated with the reactions enabling the interconversion of C1 molecules at different reduction levels present. The full reactions implementing each pathway are given in Table 3.

TABLE 3

| | | | |
|---|---|---|---|
| Reaction name | Reaction | Modification | Description |

Global modifications

| | | | |
|---|---|---|---|
| FORtppi | for_c <=> for_p | L = −1000 | Allow passive formate |
| | | | import [symbol] |
| EX_glc_{——}D_e | glc_{——}D_e <=> | L = 0 | Remove glucose input |
| FD [symbol] | b_c + nadh_c + | Add | NAD-dependent formate |
| | co2_c <=> | | dehydrogenase |
| | nad_c + for_c | | |
| formylKinase | atp_c + for_c <=> | Add | Formate activation (1 ATP) |
| | adp_c + forp_c | | |
| formylTransferase | coa_c + forp_c <=> | Add | Formate activation (1 ATP) |
| | pi_c + forcoa_c | | |
| acylAldRed | nad_c + fald_c + | Add | Conversion of formaldehyde |
| | coa_c <=> h_c + | | and formyl-CoA |
| | forcoa_c + nadh_c | | |
| MeOHDH | nad c + MeOH c <=> | Add | Methanol dehydrogenase |
| | h c + fald_c + nadh_c | | |
| hydrogenase | nad_c + h2_c <=> | Add | NAD-dependent |
| | h_c + nadh_c | | hydrogenase |

FORCE-glycolate model

| | | | |
|---|---|---|---|
| HACL | fald_c + forcoa_c <=> | Add | HACL-catalyzed reaction |
| | glyclcoa_c | | |
| glycltoaTes | h2o_c + glyclcoa_c <=> | Add | Hydrolysis of glycolyl-CoA |
| | glyclt_c + coa_c | | to glycolate |

FORCE-acetate model

| | | | |
|---|---|---|---|
| HACL | fald_c + forcoa_c <=> | Add | HACL-catalyzed reaction |
| | glyclcoa_c | | |
| glycltcoaTes | h2o_c + glyclcoa_c <=> | Add | Hydrolysis of glycolyl-CoA |
| | glyclt_c + coa_c | | to glycolate |
| ACR | h_c + nadh_c + | Add | Reduction of glycolyl-CoA |
| | glyclcoa_c <=> nad_c + | | to glycolaldehyde |
| | gcald_c + coa_c | | |
| DOR | h_c + gcald_c + | Add | Conversion of glycolaidehyde |
| | nadh_c <=> nad_c + | | and ethylene glycol |
| | ethgly_c | | |
| DDR | ethgly_c −> h2o_c + | Add | Dehydration of ethylene |
| | acald_c | | glycol |

FORCE-glyceraldehyde model

| | | | |
|---|---|---|---|
| HACL | fald_c + forcoa_c <=> | Add | HACL-catalyzed reaction |
| | glyclcoa_c | | |
| ACR | h_c + nadh_c + | Add | Reduction of glycolyl-CoA |
| | glyclcoa_c <=> nad_c + | | to glycolaldehyde |
| | gcald_c + coa_c | | |
| HACLC [symbol] | forcoa_c + gcald_c <=> | Add | HACL iteration to [symbol] 3 |
| | glycercoa_c | | |
| glycercoaTes | h2o_c + glycercoa_c <=> | Add | Hydrolysis of glyceryl-CoA |
| | glyc_R_c + coa_c | | |
| glycercoaRed | h_c + nadh_c + | Add | Reduction of glyceryl-CoA |
| | glycercoa_c <=> glyald_c + | | |
| | nad_c + coa_c | | |

RUMP model

| | | | |
|---|---|---|---|
| HPS | fald_c + ru5p_{——}D_c <=> | Add | 3-hexulose-6-phosphate |
| | h6p_c | | synthase |
| PHI | h6p_c <=> f6p_c | Add | 6-phospho-3- |
| | | | hex [symbol] loisomerase |

Serine Cycle model

| | | | |
|---|---|---|---|
| THFLig | fald_c + thf_c <=> | Add | Ligation of formaldehyde |
| | mlthf_c + h2o_c | | and tetrahydrofolate |
| SGA | ser_{——}L_c + glx_c <=> | Add | Serine-glyoxylate |
| | hpyr_c + gly_c | | aminotransferase |
| MTK | mal_{——}L_c + atp_c + | Add | Malate thiokinase |
| | coa_c −> adp_c + | | |
| | pi_c + malylcoa_c | | |
| MCL | malylcoa_c <=> accoa_c + | Add | Malyl-CoA lyase |
| | gix_c | | |

Formalase model

| | | | |
|---|---|---|---|
| FLS | 3 fald_c <=> dha_c | Add | Formolase reaction |

SACA pathway model

| | | | |
|---|---|---|---|
| GALS | 2 fald_c <=>gcald_c | Add | Glycolaldehyde synthase |
| AC [symbol] S | pi_c + gcald_c <=> | Add | Acetyl-phosphate synthase |
| | actp_c + h2o_c | | |

Reductive Glycine model

| | | | |
|---|---|---|---|
| GLYCL | nad_c + thf_c + | L = −1000 | Reversal of glycine |
| | gly_c −> nh4_c + | | cleavage |
| | mlthf_c + nadh_c + | | |
| | co2_c | | |

Formate utilization models

| | | | |
|---|---|---|---|
| EX_for_ [symbol] | for_ [symbol] −> | L = −10 | Allow formate input |
| FDH | h_c + nadh_c + | U = 0 | Prevent reutilization |
| | co2_c <=> nad_c + | | of CO₂ by direct |
| | for_c | | reduction |

Formaldehyde utilization models

| | | | |
|---|---|---|---|
| EX_fald_e | fald_e −> | L = −10 | Allow formaldehyde input |
| FDH | h_c + nadh_c + | U = 0 | Prevent reutilization |
| | co2_c <=> nad_c + | | of CO₂ by direct reduction |
| | for_c | | |
| formylKinase | atp_c + for_c <=> | B = 0 | Prevent reutilization of |
| | adp_c [symbol] forp_c | | oxidized carbon |
| formylTransferase | coa_c + forp_c <=> | B = 0 | Prevent reutilization of |
| | pi_c + forcoa_c | | oxidized carbon |

Methanol utilization models

| | | | |
|---|---|---|---|
| EX_MeOH_e | MeOH_e <=> | Add, L = −10 | Allow methanol input |
| MeOHIn | MeOH_e <=> MeOH_c | Add | Allow methanol import |
| | | | to cytoplasm (simplified) |
| FDH | [symbol] _c + nadh_c + | U = 0 | Prevent reutilization of |
| | co2_c <=> nad_c + | | CO₂ by direct reduction |
| | for_c | | |
| formylKinase | atp_c + for_c <=> | B = 0 | Prevent reutilization |
| | adp_c + forp_c | | of oxidized carbon |
| formylTransferase | coa_c + forp_c <=> | B = 0 | Prevent reutilization |
| | pi_c + forcoa_c | | of oxidized carbon |

[symbol]

indicates data missing or illegible when filed

The simulation results suggest that all the pathways that have been previously proposed to enable some form of methylotrophy in E. coli, both natural (ribulose monophosphate or RuMP, serine) and synthetic (formolase, Synthetic Acetyl-CoA or SACA, reductive glycine), are able to do so, as shown in Table 1, below.

TABLE 1

| | Carbon (g DCW/mol C) yield | | | Electron (g DCW/mol [2e−]) yield | | |
|---|---|---|---|---|---|---|
| | Formate | FormaId | Methanol | Formate | FormaId | ethanol |
| FORCE- | 3.9 | 13.0 | 19.4 | 3.9 | 6.5 | 6.5 |
| GlyceraId | | | | | | |
| Formolase | 3.8 | 12.8 | 19.1 | 3.8 | 6.4 | 6.4 |
| RuMP | 3.8 | 12.8 | 19.1 | 3.8 | 6.4 | 6.4 |
| FORCE-Ac | 3.6 | 12.1 | 18.0 | 3.6 | 6.0 | 6.0 |
| SACA | 3.6 | 12.1 | 18.0 | 3.6 | 6.0 | 6.0 |
| Reductive | 3.5 | 11.7 | 17.5 | 3.5 | 5.9 | 5.8 |
| Glycine | | | | | | |
| Serine | 3.4 | 11.2 | 16.8 | 3.4 | 5.6 | 5.6 |
| FORCE- | 3.3 | 11.1 | 16.5 | 3.3 | 5.5 | 5.8 |
| Glycolate | | | | | | |

The FORCE pathways evaluated for the conversion of non-native C1 substrates to native growth substrates glycolate, acetate, and glyceraldehyde were no exception and demonstrate another advantage of the orthogonal nature of the platform. By developing direct route(s) to compound(s) representing physiological substrates for E. coli, or any other organism, FORCE pathways can be integrated at varying or multiple metabolic nodes to capitalize on native metabolism and regulation of substrate(s) utilization, opposed to needing to engineer them. Interestingly, this in silico analysis revealed that pathways that result in the production of 3-carbon metabolites (FORCE-glyceraldehyde, formolase, RuMP) are predicted to result in the highest biomass yield on a carbon and electron basis, as shown in Table 1 above.

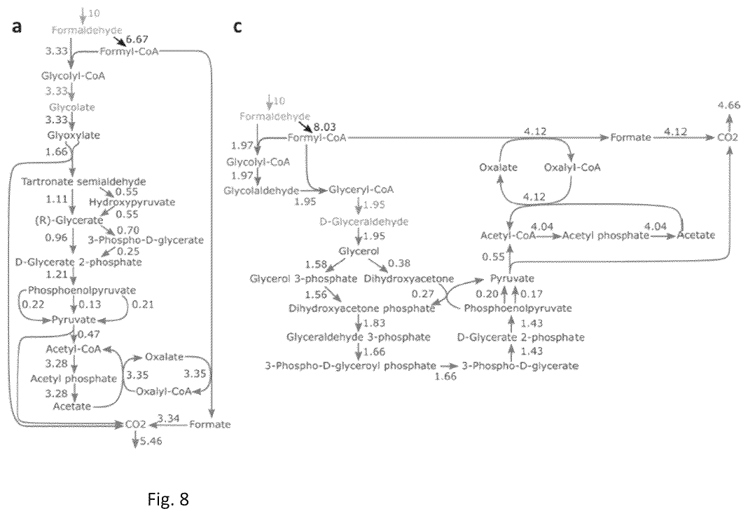

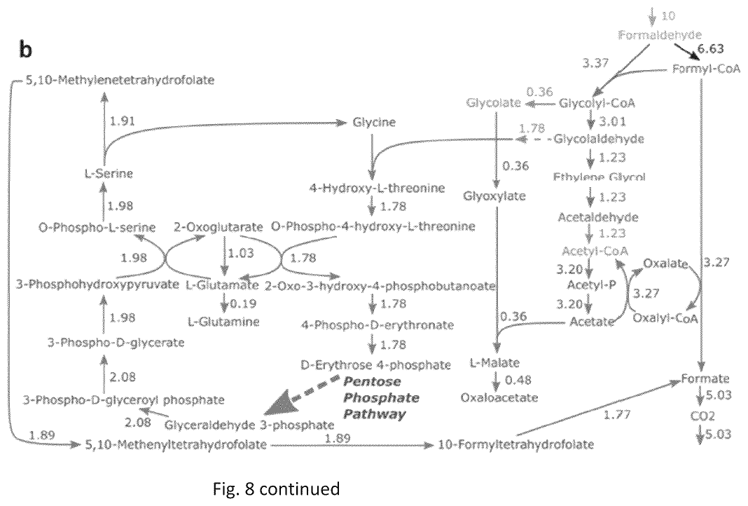

An analysis of the flux distributions of the three modeled FORCE pathways is shown in FIG. 8 and provides some insight into why the production of 3-carbon metabolites might be advantageous. As shown in FIG. 8, the FORCE pathway leading to the formation of glycolate utilizes a carbon-inefficient glycolate utilization pathway present in E. coli, which requires the decarboxylating condensation of two molecules of glyoxylate. Production of more reduced C2 metabolites, such as glycolaldehyde or acetate, is preferred to oxidized C2 in the form of glycolate. The predicted metabolism of glycolaldehyde is particularly interesting, as the model suggests a route for glycolaldehyde assimilation involving condensation with glycine and a reverse pyridoxal-5-phosphate biosynthesis pathway, ultimately resulting in pentose phosphate rearrangements to give glyceraldehyde-3-phosphate (shown in FIG. 8b). This route appears to be preferred to the assimilation of acetyl-CoA via the glyoxylate bypass based on the predicted flux distribution. As shown in FIG. 8c, direct production of glyceraldehyde from the HACL-based pathway results in the conversion of glyceraldehyde to glycerol, followed by native glycerol metabolism. As a result, pathways that lead to C3 molecules such as glyceraldehyde or dihydroxyacetone can take advantage of glycolytic reactions that result in the net production of ATP, ultimately enabling greater biomass yield. Overall, in the 3 scenarios discussed above (and illustrated in FIG. 8 and Table 1) the FORCE pathways enable the conversion of non-native C1 substrates to native multi-carbon substrates, as illustrated in FIG. 1c. Table 4 shows that the FORCE pathways also have promising characteristics on the basis of other metrics such as redox balance, ATP requirements, and number of reactions required.

TABLE 4

| | | Net | Net | | Carbon | |
|---|---|---|---|---|---|---|
| | | redox | ATP | Oxygen | yield | # Reactions |
| Pathway | Origin | (C2/C3) | (C2/C3) | sensitivity | (C2/C3) | (C2/C3) |
| This work | Engineered | +2/+4 | 0/0 | Partial | 100%/100% | 7/9 |
| RuMP | Bacterial | +5/+4 | +1/0 | None | 67%/100% | 17/10 |
| Serine | Bacterial | −1/+1 | −2/−2 | None | 200%/150% | 12/15 |
| XuMP | Eukaryal | +5/+4 | −1/−1 | None | 67%/100% | 16/15 |
| Formolase⁶ | Engineered | +5/+4 | +1/+1 | None | 67%/100% | 10/9 |
| MCC⁵ + | Engineered | +2/+7 | 0/0 | None | 100%/75% | 10/19 |
| Glyoxylate | | | | | | |
| bypass | | | | | | |
| SACA⁸ + | Engineered | +2/+7 | 0/0 | None | 100%/75% | 4/13 |
| Glyoxylate | | | | | | |
| bypass | | | | | | |

### Two-Strain Co-Culture System to Evaluate Synthetic Methylotrophy

The orthogonality of FORCE pathways to E. coli metabolism also allows for the full decoupling of the C1 conversion pathway from growth and hence for unique designs to evaluate the methylotrophic potential of the pathway. One potentially advantageous implementation might employ division of labor by separating multi-carbon compound generation and cell growth into two hosts, which would not be possible if the pathway directly interfaced with central metabolism, for example via aldose phosphates or acetyl-CoA, two common products of C1 assimilation pathways. Modularizing the system in this way allows easier analysis of the potential limitations. Using this concept, the ability for FORCE pathways to support E. coli growth on C1 substrates (such as formaldehyde, formate, and methanol) was evaluated.

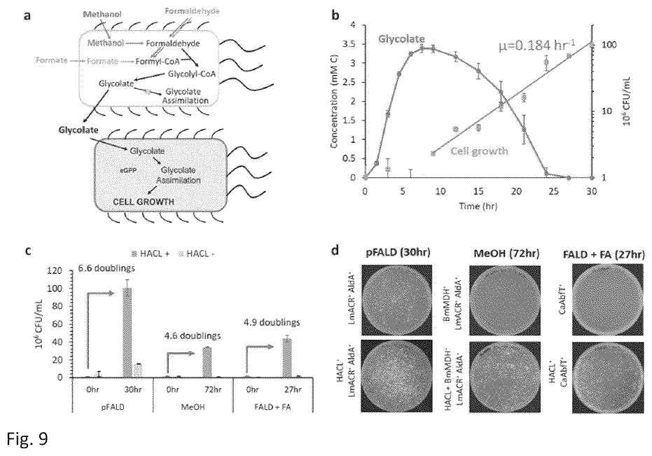

As shown in FIG. 9a, a two-strain system comprised of two engineered strains of E. coli was designed, constructed, and envisioned to work in co-culture. The first strain, referred to as the producer strain, contained constructs for the expression of the FORCE pathway for conversion of non-native C1 substrates to the native C2 growth substrate glycolate but was deficient in the ability to consume and grow on glycolate. The second strain, referred to as the sensor strain, retained the ability to grow on glycolate and additionally constitutively expressed eGFP as a signal but did not express the FORCE pathway for glycolate production. Producer and sensor strains could thus be differentiated by both selection on glycolate minimal media plates and by detection of fluorescent colonies. To assess the feasibility of different substrates, three different producer strains were devised. The producer strain for formaldehyde utilization expressed LmACR, BsmHACL, and EcAldA. The producer strain for evaluating formate utilization with formaldehyde expressed BsmHACL with CaAbfT. Finally, the producer strain for methanol utilization was the thioesterase deficient background expressing BmMdhMGA3, LmACR, BsmHACL, and CaAbfT (shown in FIG. 9a).

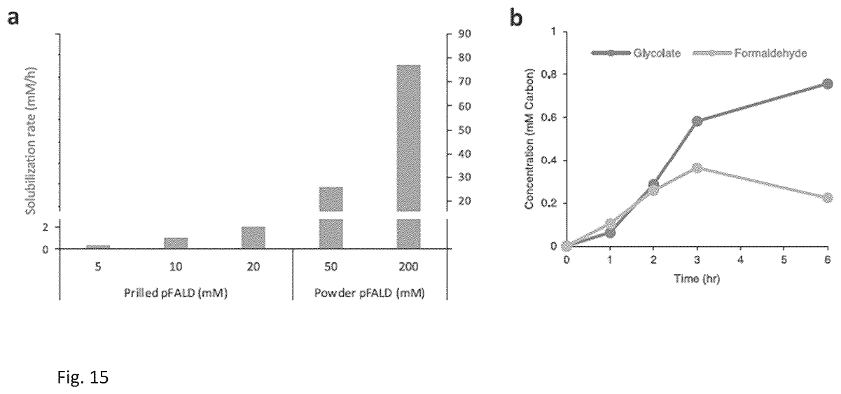

As shown in FIG. 15a, formaldehyde was the first tested substrate, although to enable growth conditions, paraformaldehyde was used. Paraformaldehyde gradually depolymerizes to give formaldehyde in aqueous media, with the ability to control the solubilization rate through the selection of particle size and concentration (FIG. 15). Referring now to FIG. 15b, this in turn enabled a system in which the formaldehyde could be kept at sub-millimolar concentrations, avoiding accumulation to toxic levels, with significant glycolate production still observed in FIG. 15. In minimal media with (para) formaldehyde (the equivalent of 5 mM) as the sole carbon substrate, growth of the sensor strain was observed as indicated by the increase in colony-forming units (CFUs) relative to a control system in which the producer strain did not express an HACL, shown in FIG. 9b and FIG. 16. Glycolate accumulated rapidly in the first 8 hours with sustained exponential growth of the sensor strain occurring after an initial lag phase. The sensor strain was found to have undergone around 6.6 doublings in 30 hours.

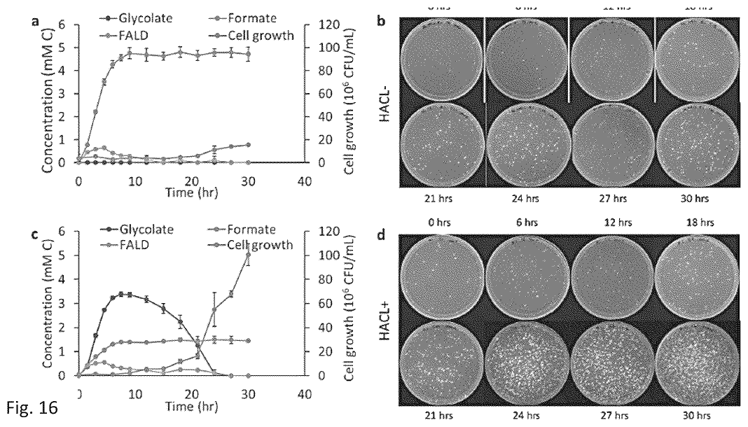

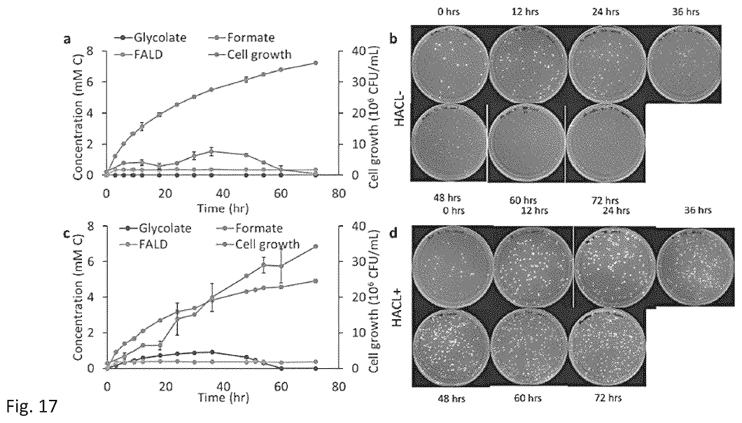

Methanol was also evaluated as a substrate for the two-strain system. FIGS. 9c, and FIG. 17 show growth of the sensor strain was observed only when the producer strain expressed BsmHACL. FIG. 17 however, compared to the case for paraformaldehyde utilization the growth kinetics of the sensor strain differed, reflecting an approximately linear increase in CFUs over time. The difference in observed dynamics might reflect the limitation imposed by the rate of glycolate production from methanol by the producer strain, analogous to the phenomenon observed in constant feed-rate fed-batch culture56. The utilization of methanol was substantially slower than the utilization of (para) formaldehyde, resulting in approximately 4.6 doublings in 72 hours.

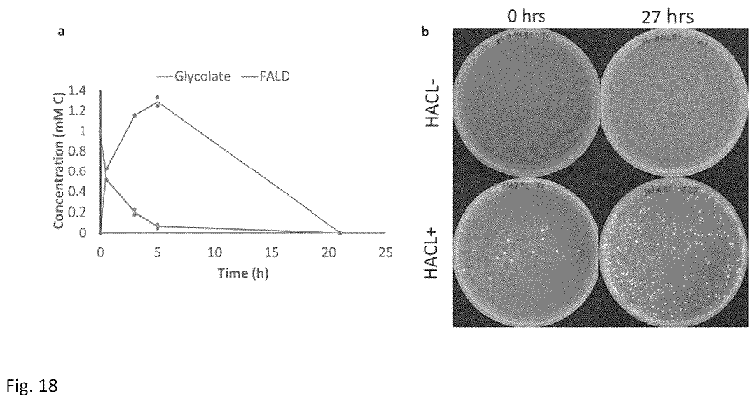

A similar experiment was performed using the 1 mM formaldehyde and 10 mM formate co-substrate system tested using resting cells. As shown in FIG. 18, and observed above, more carbon was observed in glycolate than was added as formaldehyde, indicating the incorporation of formate, FIG. 18. FIG. 9c shows growth of the sensor strain was faster than growth on methanol but did not result in as many doublings as on 5 mM (para) formaldehyde. In 27 hours, around 4.9 doublings were observed.

### DISCUSSION

While product synthesis from C1 substrates is a defining feature of FORCE pathways, they also have the potential to enable growth on non-native C1 substrates (e.g., synthetic methylotrophy) via the production of multi-carbon compounds naturally consumed by heterotrophs, such as glycolate, acetate, or glyceraldehyde. To this end, the efficacy of FORCE pathways for accomplishing synthetic methylotrophy was assessed by genome scale modeling and flux balance analysis. This analysis revealed that the FORCE pathways are comparable to or better than alternative approaches. While the current pathway performance could not support the growth of a single strain of E. coli on C1 substrates, the orthogonal nature of the pathway allowed growth, separation, and evaluation of the pathway limitations to growth on formate, formaldehyde, and methanol in separate strains of E. coli. The producer strains had to be added in excess, indicating that cell-specific improvement in pathway efficiency should enable the consolidation of FORCE pathways with growth into a single chassis. The potential for FORCE pathways to enable methylotrophy allows for bioprocess implementations more similar to traditional fermentations based on C1 as a sole carbon source. In these approaches, the substrate is used for both product synthesis and for biocatalyst production and maintenance.

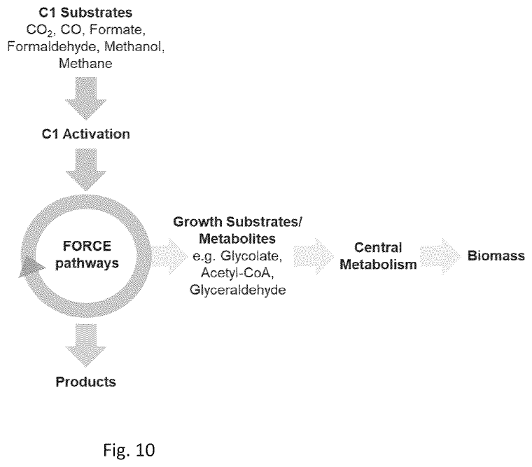

Because the FORCE pathway is the branch point for fluxes toward product synthesis and growth, there is significant potential for the facile control over flux partitioning, which is shown in FIG. 10. Fine control over these fluxes may be critical for achieving high yield bioconversions from C1, especially when carbon and energy are limited, for example in the case of formate as a sole substrate.

Further development of FORCE pathways should enable more efficient designs for synthetic methylotrophy and more diverse product synthesis, especially via pathway iteration. As an example, the primary bottleneck to be the acyloin condensation reaction of formyl-CoA was assessed with aldehydes catalyzed by HACL. The observation of formate as a byproduct throughout various implementations using reduced substrates formaldehyde and methanol is likely due to an imbalance between the rate of production of formyl-CoA and the rate of its utilization by HACL. Formyl-CoA hydrolysis has also been observed, which is likely exacerbated in vivo by the presence of endogenous thioesterases. One example approaches to address this limitation is to re-activate formate to formyl-CoA using a CoA-transferase, as we have done using the CoA-transferase CaAbfT. Identification or engineering of an HACL enzyme with better characteristics should help address this limitation. One specific example of this approach is the identification of BsmHACL, described herein. Other examples include the host-strain modifications such as the deletion of endogenous aldehyde dehydrogenases and thioesterases was explored.

Because the HACL-catalyzed condensation reaction and enzyme activity was only recently described, it is expected that further genome mining, bioprospecting, enzyme engineering, and biochemical characterization will result in the discovery of better performing variants, ultimately overcoming the pathway bottlenecks. HACL variants with well-defined chain length and functional group specificities, in combination with compatible, specific termination enzymes, should also allow for the production of specific products, analogous to what has been demonstrated with other platform pathways.

### Methods:

The methods outlined below describe the procedures and materials used to generate the particular test examples disclosed herein.

### Thermodynamic Calculations

Standard Gibbs free energies of reactions were found either from database sources (MetaCyc) or by using the eQuilibrator biochemical thermodynamics calculator. Min-max driving forces of pathways were calculated using a previously reported method implemented using MATLAB (Mathworks). The script used to perform the analysis is provided in the Supplementary Files.

### Flux Balance Analysis

Flux balance analysis was performed using the COBRA Toolbox66 for MATLAB (Mathworks) with the Gurobi solver (Gurobi Optimization, LLC). Reactions enabling the various methylotrophy pathways (as outlined in Table 3) were added or modified to the E. coli genome scale model iML151552. The limits on the substrate exchange reactions were set to 10 mmol C/g DCW/hr for all C1 substrates. The script used to perform the analysis is provided in the Supplementary Files.

### Reagents

All chemicals were obtained from Fisher Scientific Co. and Sigma-Aldrich Co. unless otherwise specified. Primers were synthesized by Integrated DNA Technologies or by Eurofins Genomics. Restriction enzymes were obtained from New England Biolabs unless otherwise specified.

### Genetic Methods

Plasmids and strains were constructed according to the methods described previously 16. Genes non-native to E. coli were codon-optimized and synthesized by GeneArt (Thermo Fisher). E. coli genes were amplified from the chromosomal DNA following standard methods. Plasmids and strains used in this study are listed in Table 2.

TABLE 2

| Host Strains/ | Description/ | |
|---|---|---|
| Plasmids | Genotype/Usage | Source |
| [symbol] | E. coli [symbol] | [symbol] |
| [symbol] | E. coli K [symbol] | [symbol] |
| [symbol] | [symbol] | [symbol] |
| [symbol] | [symbol] | This study |
| [symbol] | [symbol] | This study |
| [symbol] | [symbol] | This study |
| [symbol] | [symbol] | [symbol] |
| [symbol] | [symbol] | [symbol] |
| [symbol] | [symbol] | This study |
| [symbol] | [symbol] | This study |
| [symbol] | [symbol] | This study |
| [symbol] | [symbol] | This study |
| [symbol] | [symbol] | This study |
| [symbol] | [symbol] | [symbol] |
| [symbol] | [symbol] | This study |
| [symbol] | [symbol] | [symbol] |
| [symbol] | [symbol] | This study |
| [symbol] | [symbol] | [symbol] |
| [symbol] | [symbol] | [symbol] |
| [symbol] | [symbol] | This study |
| [symbol] | [symbol] | This study |
| [symbol] | [symbol] | [symbol] |
| [symbol] | [symbol] | This study |

[symbol]

indicates data missing or illegible when filed

### Evaluation of Core Pathway Module Using Purified Enzymes

RuHACLG390N, LmACR, and OfFrc were expressed and purified as previously described. To test the utilization of formaldehyde as the sole C1 substrate, the reaction was comprised of 50 mM KPi pH 7.4, 5 mM MgCl2, 0.1 mM TPP, 1 mM NAD+, 2 mM CoASH, 1 uM RuHACLG390N, 1 μM LmACR, and 100 mM FALD. To test the utilization of formate and formaldehyde as cosubstrates, the reaction was comprised of 50 mM KPi pH 7.4, 5 mM MgCl2, 0.1 mM TPP, 1 mM succinyl-CoA, 1 μM RuHACLG390N, 2 μM OfFrc, 100 mM sodium formate, and 100 mM formaldehyde. To test the utilization of formate as the sole C1 substrate, the reaction was comprised of 50 mM KPi pH 7.4, 5 mM MgCl2, 0.1 mM TPP, 1 mM NADH, 2 mM succinyl-CoA, 1 μM RuHACLG390N, 2 μM OfFrc, 1 μM LmACR, and 100 mM sodium formate. As a control, a reaction comprised of 50 mM KPi pH 7.4, 5 mM MgCl2, 0.1 mM TPP, 1 mM NADH, 1 mM NAD+, 2 mM succinyl-CoA, 2 mM CoASH, 2 μM BSA, 100 mM sodium formate, and 100 mM formaldehyde. The reaction volumes were 200 μL and the reactions were carried out at room temperature for 30 minutes on a rotisserie shaker. GC-MS analysis of the free acids were performed as described previously, after treating the 200 μL reaction sample with 5 μL 10 M NaOH.

To analyze the acyl-CoAs with LC-MS, the reaction was stopped by the adding 8 μL of formic acid to 200 μL reaction sample and desalted with 1 mL HyperSep C18 Cartridges (Thermo Scientific) that were primed twice with 200 μL methanol and equilibrated with 100 μL of 1 mM ammonium acetate pH 3.0. The columns were washed once with 200 μL of 1 mM ammonium acetate pH 3.0, and the acyl-CoAs were eluted in 200 μL methanol. LC-MS analysis was performed based on what has been previously described. An Agilent 6540 Q-TOF LC-MS system was equipped with a Jet-stream electrospray ionization source set to the positive ionization mode and a 100 mm×4.6 mm Kinetex 2.6 μm Polar C18 100 Å column (Phenomenex). The LC conditions were: column oven set at 40° C., injection volume of 5 μL, and 50 mM ammonium formate and methanol as the mobile phases. Compound separation was achieved using the following gradient method at a flow rate of 400 μL/min: 0 min 0% methanol; 1 min 0% methanol; 3 min 2.5% methanol; 9 min 23% methanol; 14 min 80% methanol; 16 min 80% methanol; 17 min 0% methanol. The MS conditions were: capillary voltage 3.5 kV, nozzle voltage 500 V, fragmentor voltage 150 V, with nitrogen used for nebulizing (25 psig), drying (5 L/min, 225° C.), and sheath gas (10 L/min, 400° C.). A scan range of 100-1000 m/z was used. Data was analyzed using MassHunter Qualitative Analysis B.05.00 (Agilent).

### Cell-Free Metabolic Engineering for Pathway Validation

Enzyme expression and cell extract preparation was performed as described previously. Cell-free reactions contained 50 mM KPi pH 7.4, 4 mM MgCl2, 0.1 mM TPP, 2.5 mM CoASH, 5 mM NAD+, 50 mM formaldehyde, and 0.1 mM coenzyme B12. Individual cell extract loading was around 4.4 g/L protein (⅛ of the reaction volume), and the amount of protein added to each reaction was normalized with BL21(DE3) extract to ˜26 g/L protein (¾ of the reaction volume). The reactions were incubated at room temperature for the indicated time, at which point ¼ of the reaction volume of saturated ammonium sulfate solution acidified with 1% sulfuric acid was added to stop the reactions. Samples were centrifuged at 20817×g for 15 minutes and the supernatant analyzed by HPLC as described previously.

### Resting Cell Bioconversions

Bioconversions using resting cells were performed as described previously with slight modification. The basal salts media used was M9 (6.78 g/L Na2HPO4, 3 g/L KH2PO4, 1 g/L NH4Cl, 0.5 g/L NaCl, 2 mM MgSO4, 100 μM CaCl2, and 15 μM thiamine-HCl) additionally supplemented with the micronutrient solution of Neidhardt. An overnight LB culture of each strain was used to inoculate (1%) a 250 mL flask containing 50 mL of the above media further supplemented with 20 g/L glycerol, 10 g/L tryptone, 5 g/L yeast extract, and appropriate antibiotics (50 μg/mL carbenicillin, 50 μg/mL spectinomycin). The flask cultures were incubated at 30° C. and 250 rpm in an NBS 124 Benchtop Incubator Shaker (New Brunswick Scientific Co.). After 2.5 hours, gene expression was induced by addition of 0.1 mM isopropyl β-d-1-thiogalactopyranoside (IPTG) and 0.04 mM cumate (0.2 mM IPTG and 0.1 mM cumulate was used for the experiment with formaldehyde and formate).

The cells from the above cultures were harvested by centrifugation (5000×g, 22° C., 5 min), and washed twice with the above M9 media without any carbon source. The final cell pellet was resuspended in M9 with the appropriate carbon source (˜10 OD600 with 10 mM formaldehyde or ˜5 OD600 with 1 mM formaldehyde and 10 mM formate). 5 mL of the cell suspension was added to a 25 mL Erlenmeyer flask (Corning Inc.) and topped with a foam plug. Flasks were incubated at 30° C. and 200 rpm in an NBS 124 Benchtop Incubator Shaker (New Brunswick Scientific Co.). An additional 10 mM formaldehyde was added after 1.5 hours when formaldehyde was the sole carbon source. Samples were taken after 24 hours for HPLC analysis as described previously. When 13C-labeled formaldehyde was used as the substrate, the samples were analyzed by GC-MS after extraction and derivatization as described previously.

### Fermentation Experiments

The growth media used was M9 (6.78 g/L Na2HPO4, 3 g/L KH2PO4, 1 g/L NH4Cl, 0.5 g/L NaCl, 2 mM MgSO4, 100 μM CaCl2, and 15 μM thiamine-HCl) additionally supplemented with 500 mM methanol, 10 g/L tryptone, 5 g/L yeast extract and micronutrient solution of Neidhardt. An overnight LB culture of each strain was used to inoculate (1%) a 50 mL closed-cap conical tube (Genesee Scientific Co.) containing 5 mL of the above media further supplemented with appropriate antibiotics (50 μg/mL carbenicillin, 50 μg/mL spectinomycin). After approximately 3 hours, gene expression was induced by addition of 0.04 mM isopropyl β-d-1-thiogalactopyranoside (IPTG) and 0.04 mM cumate. Tubes were incubated at 30° C. and 200 rpm in an NBS 124 Benchtop Incubator Shaker (New Brunswick Scientific Co.). Samples (100 μL) were taken every 24, 48, 72 and 96 hours after inoculation for OD600 measurement and HPLC analysis as described previously. When 13C-methanol was used as the substrate, the samples were analyzed by GC-MS after extraction and derivatization as described previously.

Two-Strain E. coli System for Growth with Formaldehyde as the Sole Carbon Source

Two-strain experiments were conducted using strains cultured and induced as described previously using M9 medium. The induced cells were resuspended to an initial concentration of 3*109 CFU (colony forming unit)/mL (equivalent to OD600 of ˜5) in M9 medium. 20 mL of the suspension was added into 25 mL flask containing 3 mg paraformaldehyde (equivalent to 5 mM), or 10 mL of the suspension was added into 25 mL flask with the addition of 500 mM methanol, or 1 mM formaldehyde and 10 mM sodium formate. A second E. coli strain, AC763, capable of consuming glycolate, was added to an initial concentration of 5*10⁶ CFU/mL (equivalent to OD600 of ˜0.005). AC763 additionally harbored a chromosomal copy of constitutively expressed eGFP to assist in distinguishing the two strains. Prior to its addition to the culture, AC763 was pre-grown in 25 mL Erlenmeyer flasks (from a single colony inoculation) at 200 rpm and 30° C. for 24 hours in 5 mL of the above M9 minimal media supplemented with 5 g/L glycolate and 2 g/L tryptone. Cells were then centrifuged (5000×g, 22° C., 5 min), washed twice with the media supplemented with 5 g/L glycolate, and resuspended to an optical density of ˜0.05. Following 24 hours of incubation at 200 rpm and 30° C. (5 mL in 25 mL Erlenmeyer flasks), cells were centrifuged (5000×g, 22° C.), washed twice with media without any carbon source and an appropriate volume added to the two-strain system. The flasks containing both strains were further incubated at 200 rpm and 30° C. Samples were taken at various times for HPLC and cell growth analysis. Colony forming units per mL of culture was utilized as a measurement of cell growth. Appropriate volumes of culture were diluted in the above-described minimal media without any carbon source and 50 μL of various dilutions plated on minimal media plates containing 2.5 g/L glycolate. Following plate incubation at 37° C., colonies were counted manually, aided by visualization using a blue-light transilluminator (Vernier, Beaverton, OR) to illuminate the eGFP expressing strain AC763.

As noted previously, it will be appreciated by those skilled in the art that while the disclosure has been described above in connection with particular embodiments and examples, the disclosure is not necessarily so limited, and that numerous other embodiments, examples, uses, modifications and departures from the embodiments, examples and uses are intended to be encompassed by the claims attached hereto.

### EXAMPLES

### Example 1: Strategy Used to Identify Enzymes with Similar Structure and/or Function Based on Sequence Similarity

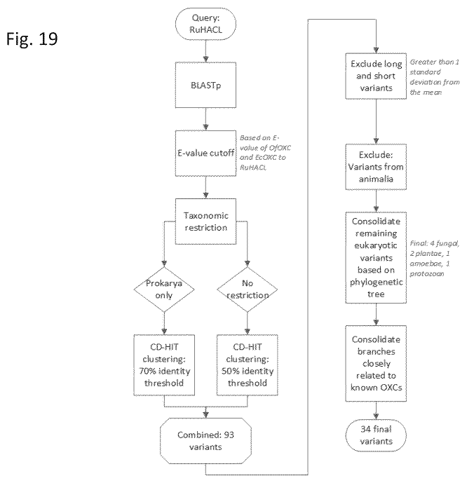

The purpose of this example is to provide an overview of workflow used to identify enzyme variants with desired activity starting from reference enzyme as query. In this example, 2-hydroxyacyl-CoA lyase, HACL from Rhodospirillales bacterium URHD0017 (RuHACL) is used as a starting query for identification of the first round 2-hydroxyacyl-CoA synthase (HACS) variants. Protein BLAST (pBLAST) is used with E-value cutoff based on the E-value between RuHACL and oxalyl-CoA decarboxylase, OXC from Escherichia coli (EcOXC) and Oxalobacter formigenes (OfOXC) (FIG. 19). To down select representative variants by clustering query results into families with similar sequences, we used CD-HIT web server (Huang et al. Bioinformatics, (2010). 26:680). More lenient restriction of 70% identity threshold was imposed for genes from prokaryotic origin whereas 50% was used for no taxonomic restriction (FIG. 19). Clustering and picking representative genes using CD-HIT gave 93 HACS variants similar to RuHACL. Further curations from the list, including elimination of too long or too short sequences and variants from animalia, which is not likely to be expressed well in E. coli. As a result, we determined 34 remaining variants after the curations to be the initial round of HACS variants for synthesis and testing in E. coli as a host (FIG. 19). The selected genes were then codon-optimized for expression in E. coli and synthesized in collaboration with Joint Genome Institute (JGI). 5 variants failed during synthesis, which gave 29 first round JGI HACS variants (JGI1 to JGI29) (Table 5).

TABLE 5
List of 2-hydroxyacyl-CoA (HACS) variants (JGI) identified
by selecting representative genes from gene clusters with
sequence similarity using RuHACL as reference enzyme.

| | GenBank |
|---|---|
| JGI# | Accession Number |
| 1 | XP_012756082.1 |
| 2 | TMK01573.1 |
| 3 | PYM26381.1 |
| 4 | EEG70177.1 |
| 5 | MBH80817.1 |
| 6 | WP_030891887.1 |
| 7 | AGK93615.1 |
| 8 | MAX57815.1 |
| 9 | WP_068916287.1 |
| 10 | WP_062165271.1 |
| 11 | MBB43458.1 |
| 12 | PCJ72347.1 |
| 13 | TMQ19149.1 |
| 14 | MAX11513.1 |
| 15 | HAK63664.1 |
| 16 | MBG92919.1 |
| 17 | PZC46201.1 |
| 18 | MBB84818.1 |
| 19 | OGA51379.1 |
| 20 | PWB41796.1 |
| 21 | MAE93843.1 |
| 22 | OGP60024.1 |
| 23 | OWB57166.1 |
| 24 | KXN72624.1 |
| 25 | PVU86112.1 |
| 26 | ORZ16580.1 |
| 27 | XP_005644825.1 |
| 28 | KZV27770.1 |
| 29 | EJY87672.1 |

### Example 2: Establishing High Throughput Platform for Screening First Round HACS Variants for C1-C1 Condensation

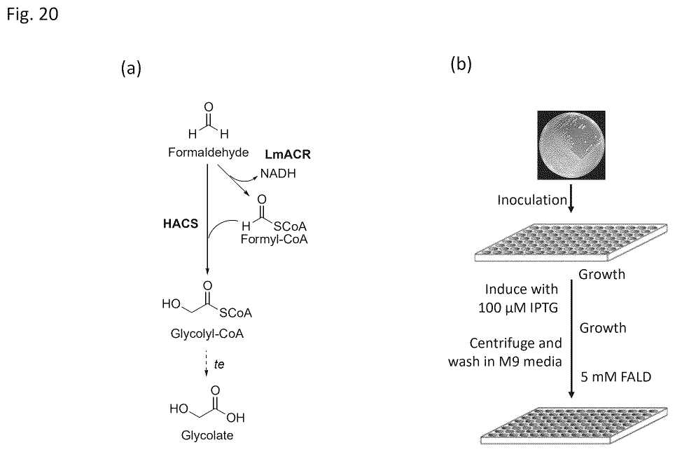

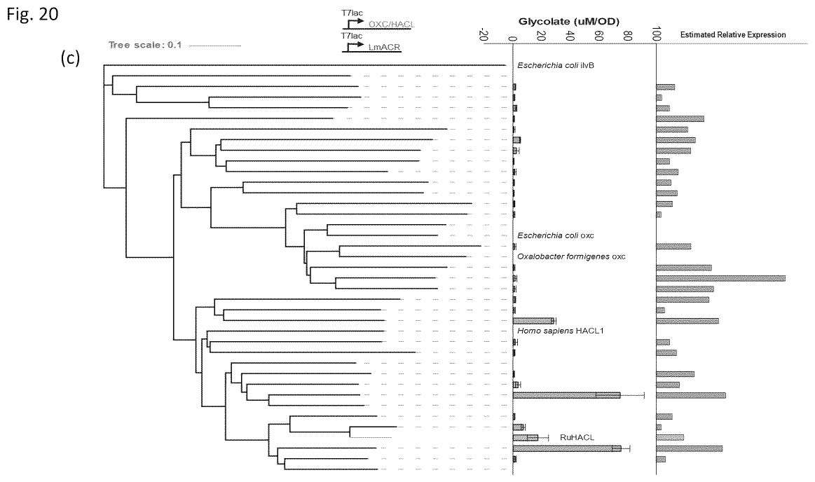

The purpose of this example is to demonstrate high throughput platform for screening 2-hydroxyacyl-CoA synthase (HACS) variants in vivo. We used glycolic acid (glycolate) productivity per cell density (uM glycolate/OD600) as indicator of HACS activity. Glycolate can be produced from formaldehyde as sole carbon source in the presence of active HACS variant and acyl-CoA reductase from Listeria monocytogenes (LmACR) (FIG. 20A). LmACR is shown to be capable of catalyzing oxidation reaction from formaldehyde to formyl-CoA (Chou, A., et al. Nat. Chem. Biol. 15:900-906 (2019)). HACS condenses formaldehyde and formyl-CoA to form glycolyl-CoA, which can then be hydrolyzed to glycolate via native thioesterase activities (FIG. 20A).

To prototype glycolate production pathway from formaldehyde in vivo, we constructed vectors overexpress various HACS candidates and LmACR, with both under control of the IPTG-inducible T7 promoter in pCDFDuet-1 and pETDuet-1, respectively (FIG. 20C). As a host for these vectors, we used an engineered strain of E. coli based on MG1655 (DE3) with knockouts for formaldehyde (ΔfrmA) and formate (ΔfdhF ΔfdnG ΔfdoG) oxidation as well as for glycolate utilization (ΔglcD)), which we expected could compete or interfere with the analysis of our pathway.

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.2 mL of the above media further supplemented with 20 g/L glycerol, 10 g/L tryptone, and 5 g/L yeast extract. A single colony of the desired strain was cultivated overnight (14-16 hrs) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside (IPTG)) were added. Plates were incubated for a total of 24 hrs. post-inoculation (FIG. 20B).

Cells from the above pre-cultures were then centrifuged (4000 rpm, 22° C.), washed with the above minimal media without any carbon source, and resuspended with 1 mL of above minimal media containing indicated amounts of carbon source. 5 mM formaldehyde was added at 0 hr. and were incubated at 30° C. and 1000 rpm in Digital Microplate Shaker (Fisher Scientific). After incubation at 30° C. for 3 hours, the cells were pelleted by centrifugation and the supernatant analyzed by HPLC or GC-MS as described below. Cell pellets harvested after bioconversion were resuspended to 20OD in B-PER® Bacterial Protein Extraction Reagent (Thermo Fisher) supplemented with 0.1 mg/mL chicken egg white lysozyme (Fisher) and 5 U/mL Benzonase® nuclease (Sigma) for cell lysis. After incubation in room temperature for 15 minutes, 100 μL of each cell lysate was transferred to 1.5 mL microcentrifuge tubes for centrifugation at 15,000×g for 5 minutes. The soluble cell lysates obtained from the supernatant were analyzed using SDS-PAGE. Relative HACS expression was estimated by band area in the protein gel image.

Quantification of product and substrate concentrations (formic acid, formaldehyde, and glycolic acid) were determined via HPLC using a Shimadzu Prominence SIL 20 system (Shimadzu Scientific Instruments, Inc., Columbia, MD) equipped with a refractive index detector and an HPX-87H organic acid column (Bio-Rad, Hercules, CA) with operating conditions to optimize peak separation (0.3 ml/min flowrate, 30 mM H₂SO₄ mobile phase, column temperature 42° C.). Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 μm film thickness, 30 m length).

The screening of first round HACS variants shows that three variants out of 29 (JGI15, 19, 20) demonstrating better glycolate productivity and relative HACS expression than the starting reference, RuHACL (FIG. 20C). JG115 and JGI20 are the two best candidates showing more than 3-fold improvement in glycolate productivity and are chosen for further analysis.

### Example 3: Testing High Performing Variants Under Various C1-C1 Condensation Platforms

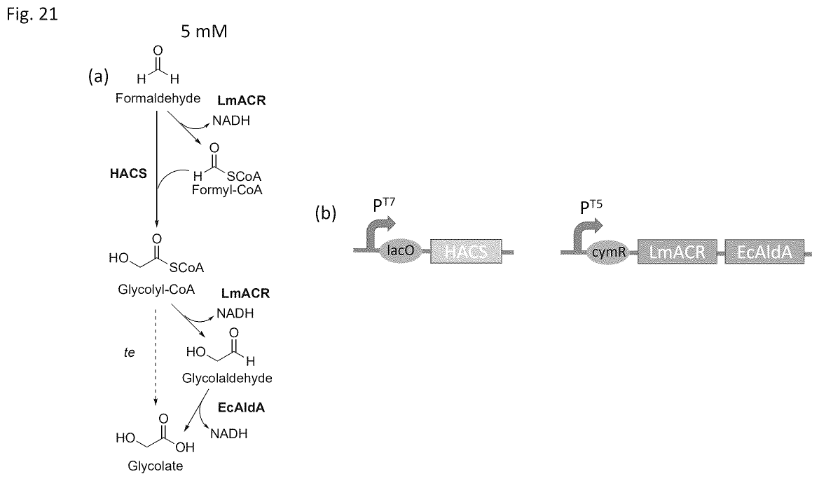

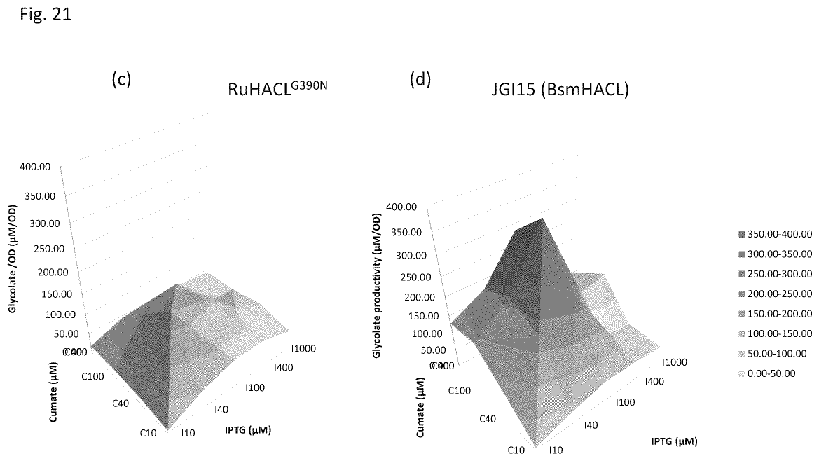

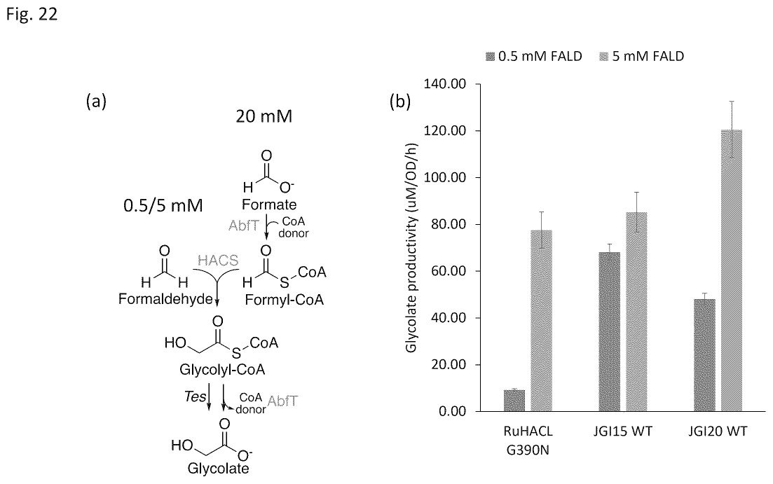

The purpose of this example is to demonstrate analysis on the two high performing HACS variants (JGI15 and JG120) in comparison with the reference enzyme, RuHACL. We used glycolic acid (glycolate) productivity per cell density (uM glycolate/OD600) as indicator of HACS activity. Two different enzymatic routes for glycolate synthesis are explored. The first pathway (pathway 1) is similar to the pathway used for initial screening in Example 2 with addition of an extra gene, aldehyde dehydrogenase aldA from Escherichia coli (EcAldA) overexpressed to drive flux from glycolaldehyde to glycolate (FIG. 21A). In addition, HACS and LmACR-AldA are controlled under independent inducible promoters to investigate the impact of varying relative gene expressions (FIG. 21B). The second pathway (pathway 2) involves independent fluxes of formaldehyde and formyl-CoA allowing assessment of the enzyme activity with response to changing formaldehyde concentration only, while maintaining constant formyl-CoA flux. Formyl-CoA is generated by formic acid (formate) catalyzed by the acyl-CoA transferase from Clostridium aminobutyricum (CaAbfT) (FIG. 22A).

For the in vivo prototyping, we engineered vectors to independently control expression of HACS variants and the LmACR-EcAldA (pathway 1, FIG. 21A) or CaAbfT (pathway 2, FIG. 22A), with HACS under control of the IPTG-inducible T7 promoter in pCDFDuet-1 and LmACR-EcAldA or CaAbfT under control of a cumate-inducible T5 promoter in pETDuet-1 (FIG. 21B). As a host for these vectors, we used an engineered strain of E. coli based on MG1655(DE3) with knockouts for formaldehyde (ΔfrmA) and formate (ΔfdhF ΔfdnG ΔfdoG) oxidation as well as for glycolate utilization (ΔglcD)), which we expected could compete or interfere with the analysis of our pathway.

In vivo product synthesis was conducted using M9 minimal media (6.78 g/L Na₂HPO₄, 3 g/L KH₂PO₄, 1 g/L NH₄Cl, 0.5 g/L NaCl, 2 mM MgSO₄, 100 μM CaCl₂), and 15 μM thiamine-HCl) unless otherwise stated. Cells were initially grown in 96-deep well plates (USA Scientific, Ocala, FL) containing 0.2 mL of the above media further supplemented with 20 g/L glycerol, 10 g/L tryptone, and 5 g/L yeast extract. A single colony of the desired strain was cultivated overnight (14-16 hrs) in LB medium with appropriate antibiotics and used as the inoculum (1%). Antibiotics (100 μg/mL carbenicillin, 100 μg/mL spectinomycin) were included when appropriate. Cultures were then incubated at 30° C. and 1000 rpm in a Digital Microplate Shaker (Fisher Scientific) until an OD600 of ˜0.4 was reached, at which point appropriate amounts of inducer(s) (isopropyl β-D-1-thiogalactopyranoside (IPTG) and cumate) were added. Plates were incubated for a total of 24 hrs. post-inoculation (FIG. 20B).

Cells from the above pre-cultures were then centrifuged (4000 rpm, 22° C.), washed with the above minimal media without any carbon source, and resuspended with 1 mL of above minimal media containing indicated amounts of carbon source. 5 mM formaldehyde only for LmACR-EcAldA co-expression (FIG. 21A), 0.5/5 mM formaldehyde and 20 mM formate for CaAbfT co-expression (FIG. 22A) was added at 0 hr and were incubated at 30° C. and 1000 rpm in Digital Microplate Shaker (Fisher Scientific). The cells were harvested after 3 hours for LmACR-EcAldA co-expression and 1 hour for CaAbfT co-expression by centrifugation and the supernatant analyzed by HPLC or GC-MS as described in EXAMPLE 2.

When 5 mM formaldehyde was used as sole carbon source, JGI15 (FIG. 21C) performs 2.5-fold better than RuHACL (FIG. 21D) under optimal inducer concentrations (relative gene expressions), based on glycolate productivity (μM/OD600) in 3 hours. When formaldehyde and formate are co-fed, JGI15 outperforms RuHACL and JGI20 in a substantial margin (7-fold and 1.5-fold, respectively) under low formaldehyde availability (0.5 mM) indicating better affinity (low Kₘ) of JGI15 with formaldehyde (FIG. 4B). On the other hand, JGI20 shows better glycolate productivity under high formaldehyde concentration (5 mM) indicating better turnover (high k_{cat}) of this variant.

### Example 4: Kinetic Characterization of High Performing HACS Variants

The purpose of this example is to demonstrate the kinetic characterization of the high performing HACS variants (JGI15, JG120, JGI23 and JG124) from the first-round homologs using in vitro kinetic assay with purified enzymes. The kinetic assay was performed with a coupled reaction providing formyl-CoA from formate catalyzed by CoA transferase CaAbfT using acetyl-CoA as a CoA donor.

Expression of selected enzyme variants was achieved using plasmid-based gene expression either constructed by Joint Genome Institute (JGI) for HACS variants (JGI15, 20, 23 and 24) or by cloning the desired gene(s) into pCDFDuet-1 (Novagen, Darmstadt, Germany) digested with appropriate restriction enzymes and by utilizing In-Fusion cloning technology (Clontech Laboratories, Inc., Mountain View, CA). Linear DNA fragments for insertion were created by gene synthesis of the codon optimized gene. Genes were synthesized by GeneArt (Life Technologies, Carlsbad, CA) or Twist (Twist Biosciences). Resulting In-Fusion reaction products were used to transform E. coli Stellar cells (Clontech Laboratories, Inc., Mountain View, CA), and clones identified by PCR screening were further confirmed by DNA sequencing.

Overnight cultures of the expression strains were grown in LB, which were used to inoculate 25 mL TB medium in a 250 mL baffled flask at 1 v/v % (250 μL). The culture was grown at 30° C. and 250 rpm in an orbital shaker until OD550 reached 0.4-0.6, at which point expression was induced with 0.1 mM IPTG. 24 hours post inoculation, cells were harvested by centrifugation. The cell pellets were washed once with cold 9 g/L NaCl solution and stored at −80° C. until needed. Antibiotics were included where appropriate at the following concentrations: carbenicillin (50 μg/mL), and spectinomycin (50 μg/mL).

For protein purification, E. coli cell pellets expressing the desired his-tagged enzymes were prepared as described above. The frozen cell pellets were resuspended in cold lysis buffer (50 mM NaPi pH 7.4, 300 mM NaCl, 10 mM imidazole, 0.1% Triton-X 100) to an approximate OD550 of 40, to which 1 mg/mL of lysozyme and 250 U of Benzonase nuclease was added. The mixture was further treated by sonication on ice using a Branson Sonifier 250 (5 minutes with a 25% duty cycle and output control set at 3), and centrifuged at 7500×g for 15 minutes at 4° C. The supernatant was applied to a chromatography column containing 1 mL TALON metal affinity resin (Clontech Laboratories, Inc., Mountain View, CA), which had been pre-equilibrated with the lysis buffer. The column was then washed first with 10 mL of the lysis buffer and then twice with 20 mL of wash buffer (50 mM NaPi pH 7.4, 300 mM NaCl, 20 mM imidazole). The his-tagged protein of interest was eluted with 1-2 applications of 4 mL elution buffer (50 mM NaPi pH 7.4, 300 mM NaCl, 250 mM imidazole). The eluate was collected and applied to a 10,000 MWCO Amicon ultrafiltration centrifugal device (Millipore, Billerica, MA), and the concentrate (˜100 μL) was washed twice with 4 mL of 50 mM KPi pH 7.4 for desalting. Protein concentrations were estimated by the Bradford method. Purified protein was saved in 20 μL aliquots at −80° C. until needed.

SDS-PAGE was performed using NuPAGE 12% Bis-Tris Protein Gels with SDS running buffer and stained with SimplyBlue SafeStain according to manufacturer protocols (ThermoFisher Scientific, Waltham, MA).

In vitro kinetic assay was comprised of 100 mM KPi pH 6.9, 10 mM MgCl2, 0.15 mM TPP, 2 mM acetyl-CoA, 1 μM CaAbfT, 0.25 μM HACS variants, and 20 mM sodium formate. Reactions were incubated at room temperature for 3 min to convert formate to formyl-CoA, and then specific concentration of aldehyde (specifically acetaldehyde or propionaldehyde here) was added to the reaction. After incubating another 3 min, 1/20 of the reaction volume of 10 M NaOH solution was added to terminate the reactions. After 30 min hydrolysis, 1/20 of the reaction volume of 10 N H₂SO₄ was added to neutralize the pH. Samples were centrifuged at 20817×g for 15 minutes and the supernatant analyzed by GC-MS as described below.

For this analysis, 0.15 of the reaction volume of internal standard methyl succinate was added to the samples. The resulting sample was extracted into 4 mL ethyl acetate by vigorous vortexing for 20 min. The organic phase was separated and evaporated to dryness under a stream of nitrogen. The residue was dissolved in 30 μL pyridine and 30 μL N,O-Bis(trimethylsilyl)trifluoroacetamide (BSTFA) and incubated at 60° C. for 15 minutes. Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 μm film thickness, 30 m length). Samples were analyzed by GC (1 μL injection with a 20:1 split ratio) using helium as the carrier gas at a flowrate of 1.5 mL/min and the following temperature profile: initial 90° C. for 3 min; ramp at 15° C./min to 170° C.; ramp at 20° C./min to 300° C. and hold for 8 min. The injector and detector temperature were 250° C. and 350° C., respectively.

As hypothesized from Example 3 (FIG. 22B), JGI15 had lower Kₘ with lower k_{cat} with respect to formaldehyde in comparison with JGI20 (Table 8). With longer chain aldehydes JGI15 and JGI20 has lower Kₘ (sub millimolar) to acetaldehyde and propionaldehyde compared to JGI23 and JGI24, while also lower K_{cat} as well. Which indicates they have stronger affinity to the aldehyde and lower enzymatic activity. JGI23 and JGI24 are promising as they have better activity (higher K_{cat}) which have a good potential to work better in vivo by improve their affinity to substrates (lower Kₘ). Similarly, AcHACL as lower Km with acetone while JGI15 as better Kcat (Table 8).

TABLE 8
Apparent kinetic parameters for the 2-hydroxyacyl-CoA synthase (HACS)
variants with various aldehydes and ketones as substrates.

| | | | |
|---|---|---|---|
| | K_{cat, app} | K_{m, app} | K_{cat, app}/K_{m, app} |
| Substrate | (s⁻¹) | (mM) | (M⁻¹ s⁻¹) |

JGI15

| | | | |
|---|---|---|---|
| Formaldehyde | 5.98 | 3.87 | 6.5 × 10² |
| Acetaldehyde | 22.50 ± 1.93 | 0.29 ± 0.02 | 7.8 × 10⁴ |
| Propionaldehyde | 11.08 ± 0.02 | 0.20 ± 0.04 | 5.5 × 10⁴ |
| Acetone | 6.8 | 554 | 12.3 |

JGI20

| | | | |
|---|---|---|---|
| Formaldehyde | 9.53 | 21.43 | 4.4 × 10² |
| Acetaldehyde | 39.44 ± 3.95 | 0.93 ± 0.15 | 4.2 × 10⁴ |
| Propionaldehyde | 17.35 ± 1.97 | 0.42 ± 0.06 | 4.1 × 10⁴ |

JGI23

| | | | |
|---|---|---|---|
| Acetaldehyde | 83.04 | 7.44 | 1.1 × 10⁴ |
| Propionaldehyde | 29.29 ± 4.70 | 5.29 ± 0.44 | 5.5 × 10³ |

JGI24

| | | | |
|---|---|---|---|
| Acetaldehyde | 40.07 | 7.96 | 5.0 × 10³ |
| Propionaldehyde | 26.17 | 4.57 | 5.7 × 10³ |

AcHACL

| | | | |
|---|---|---|---|
| Acetone | 0.42 | 72 | 5.8 |

RuHACL¹

| | | | |
|---|---|---|---|
| Formaldehyde | 3.3 ± 0.4 | 29 ± 8 | 1.1 × 10² |
| Propionaldehyde | 4.7 ± 0.4 | 16 ± 4 | 3.0 × 10² |
| Acetone | 2.7 ± 0.8 | 1600 ± 700 | 1.7 ± 0.9 |

¹Chou et al. Nat Chem Biol 15, 906-919 (2019)

### Example 5: Modeling Protein Structure of High Performing HACS Variants and Understanding Key Catalytic Residues Through Structural Analysis

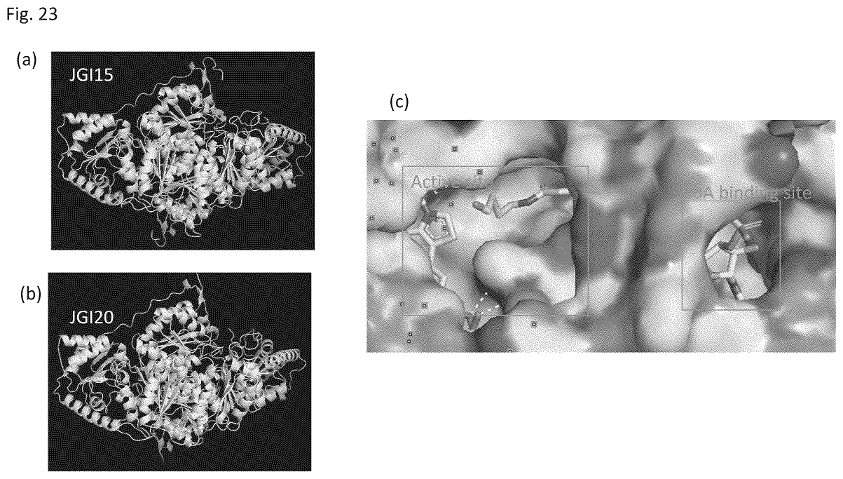

This example demonstrates the analysis of recombinant high performing HACS variants (JGI15 and JGI20) using protein structure analysis and alanine scanning method. The full dimeric structure of JGI15 (FIG. 23A) and JG120 (FIG. 23B) are modeled using AlphaFold (Jumper et al. Nature 596:583-589 (2021)) in the ColabFold platform (Mirdita et al. Nature Methods 19:679-682 (2022)). The models are aligned with the crystal structure of oxalyl-CoA decarboxylase from Oxalobacter formigenes in complex with formyl-CoA (PDB code: 2JI8) (Berthold et al. Structure 15:853-861 (2007)) to understand orientation of two key ligands, thiamine diphosphate (TPP) and formyl-CoA, in the active site. The structures are highly similar with root-mean-square distance (RMSD) value of 1.185 Å and 0.981 Å for JGI15 and JGI20, respectively. The active site (where TPP and the formyl residue of formyl-CoA interface for catalysis) and the CoA binding site are both exposed to the solvent indicating correct orientation of ligands docked on to the JGI15 and JGI20 structures (FIG. 23C).

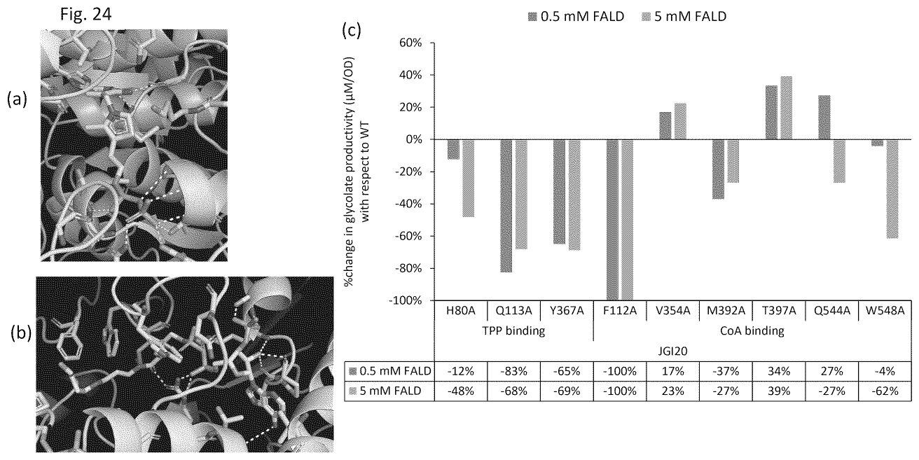

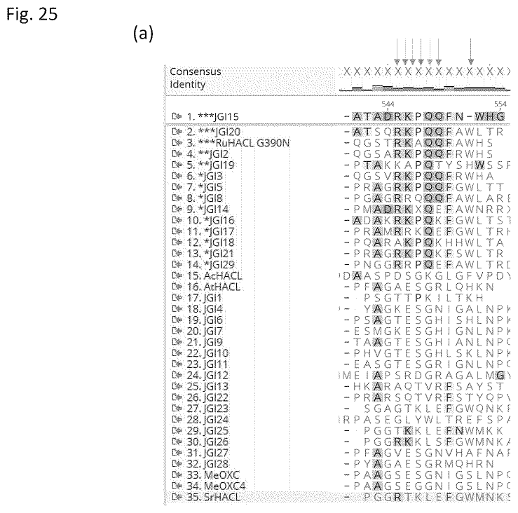

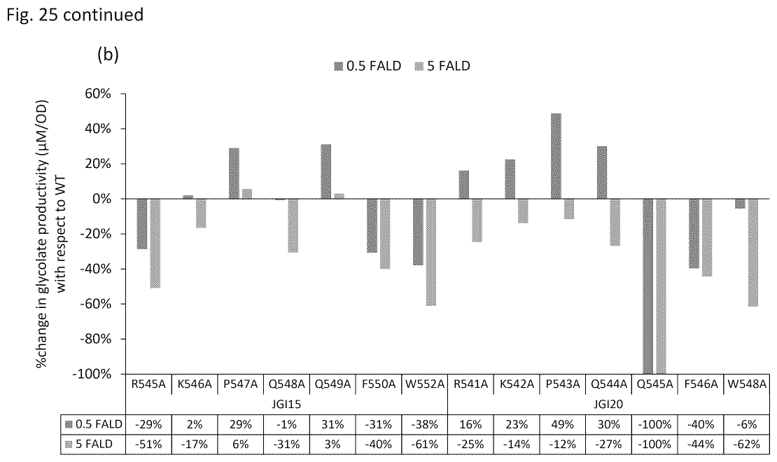

To understand the specific amino acid (AA) residues responsible for the catalytic activity and substrate binding, we have selected all AA residue within 3.5 Å from either TPP (FIG. 24A) or formyl-CoA (FIG. 24B) using JG120 as a reference protein (TABLE 7). Then, we have selected AA residues that are not conserved among all first round JGI variants (30 including RuHACL) and are unique to variants that have C1-C1 condensation activities. 3 AA residues from TPP binding site (H80, Q113 and Y367 from JGI20) and 6 from CoA binding site (F112, V354, M392, T397, Q544 and W548) are selected as a result (FIG. 24C). Q544 and W548 are located at the c-terminal end of JGI20 which was shown to form closing loop covering the active site in other similar proteins, such as 2-hydroxyacyl-CoA lyase from Actinomycetospora chiangmaiensis (AcHACL) (Zahn et al. J. Biol. Chem. 298 (1) 101522 (2022)). It was found that active HACS variants have conserved residues of “RKPQQF-W” in this region while others do not (FIG. 25A). As a result, we decided to explore the importance of conserved residues only among active variants in near the active site and the c-terminal closing loop using alanine scanning method (Morrison and Weiss, Curr Opin Chem Biol. 5 (3): 302-7 (2001)) to identify the key catalytic residues among them.

TABLE 7
Active site residues (3.5 Å within TPP and formyl-CoA
based on the AlphaFold structure of JGI20) and corresponding
residues of high performing variants on C1-C1 condensation.

| | JGI20 | JGI15 | JGIH25 | JGIH26 | JGIH30 | JGIH41 | JGIH61 | JGIH65 |
|---|---|---|---|---|---|---|---|---|
| TPP | V26 | V | V | V | V | V | V | V |
| binding | E50 | E | E | E | E | E | E | E |
| | V73 | V | V | V | V | V | V | V |
| | G77 | G | G | G | G | G | G | G |
| | H80 | H | H | H | H | H | H | N |
| | Q113* | Q | Q | Q | Q | Q | Q | Q |
| | Y367* | Y | Y | Y | Y | Y | Y | Y |
| | T391 | T | T | T | T | T | T | T |
| | G414 | G | G | G | G | G | G | G |
| | M416 | M | M | M | M | M | M | M |
| | D441 | D | D | D | D | D | D | D |
| | S442 | S | S | S | S | S | A | G |
| | A443 | A | A | A | A | A | A | A |
| | N469 | N | N | N | N | N | N | N |
| | G471 | G | G | G | G | G | G | G |
| CoA | F112* | F | F | F | F | F | F | F |
| binding | A253 | G | S | S | A | G | S | A |
| | P254 | G | P | P | P | A | P | A |
| | R256 | R | R | R | R | R | R | R |
| | S257 | S | S | S | S | S | T | S |
| | W275 | W | W | W | W | W | W | W |
| | M276 | I | I | I | I | I | I | M |
| | V354 | V | V | V | V | T | S | V |
| | M392* | M | M | M | M | M | M | M |
| | R396 | R | R | R | R | R | R | R |
| | T397 | T | T | T | T | T | T | T |
| | Q544 | Q | Q | Q | Q | Q | Q | Q |
| | W548 | W | W | W | W | W | W | W |
| c- | L549 | H | L | L | L | L | H | L |
| terminal | T550 | G | T | T | T | T | T | T |
| end | R551 | — | R | R | R | T | P | R |
| | — | — | — | — | — | — | TN | TNE |

Residues in bold indicates unconserved residues among active variants. Residues with asterisk (*) indicate key catalytic residues that are hypothesized to distinguish between HACS and OXC.

JGI15 and JGI20 mutants were prepared by cloning wild type JGI15 and JGI20 into the vector pUC19 (Clontech Laboratories, Inc., Mountain View, CA). Primers containing the desired mutation were designed following the ‘In Vivo assembly’ (IVA) protocol for mutagenesis (Garcia-Nafria et al., Sci. Rep. 6, 12. 2016). PCR products containing the mutations were generated following the IVA protocol and used to transform E. coli Stellar cells (Clontech Laboratories). The desired mutant sequence was confirmed by DNA sequencing. The mutant genes were then cloned into final expression vector (pCDFDuet-1) using restriction enzyme digestion and ligation. HACS activities of the mutants are examined in an identical format as pathway 2 described in EXAMPLE 3 (FIG. 22A) with 0.5 mM formaldehyde and 20 mM formate as carbon sources.

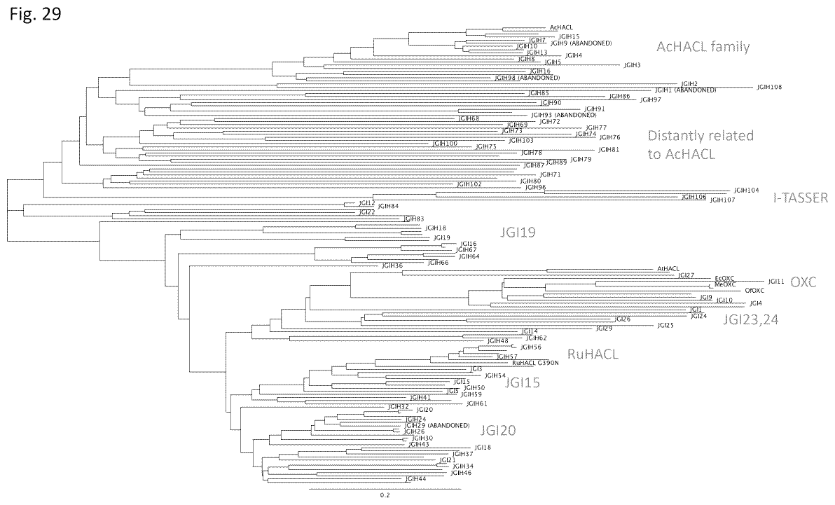

The alanine scanning results on active site residues show that Glutamine113 (Q113) and Tyrosine367 (Y367) from TPP binding and Phenylalanine112 (F112) and Methionone392 (M392) from CoA binding are important residues for the HACS activity on formaldehyde-formyl-CoA condensation (FIG. 24C). Q113 was shown to have a key catalytic function in other 2-hydroxyacyl-CoA synthases such as AcHACL (Zahn et al. J. Biol. Chem. 298 (1) 101522 (2022)). In addition, mutagenesis of F112 and Q113 showed abolition of HACS activities in the previous studies on RuHACL (Chou, A., et al. Nat. Chem. Biol. 15:900-906 (2019)). It was also found that known oxalyl-CoA decarboxylase (OXC) variants including genes from E. coli, O. formigenes and Methylorubrum extorquens have conserved Tyrosine-glutamic acid (YE) residues in the place of F112 Q113. All variants from first round HACS variants that have OXC-like “YE” residues (JGI4, 6, 7, 9, 10, 11) did not have any glycolate production from formaldehyde and are clustered together with OXCs in the phylogenetic analysis (FIG. 29). Therefore, “FQ” and “YE” could be the key catalytic residues that distinguish HACS and OXC type of enzymes. Interestingly, the two additional residues (Y367 and M392) found to be important for catalytic function (FIG. 24C) are also only conserved among HACS but not OXC (Table 7). OXC-type enzymes have unconserved residues in the position corresponding to Y367 and have conserved leucine (L) residue in the place of M392. Therefore, the two residues might also play important role in the catalytic function distinguishing HACS and OXC activities.

With exception of Q545A of JG120, none of the c-terminal residues in JGI15 and JGI20 abolished activity from point mutagenesis to alanine (FIG. 25B). It is expected as c-terminal end of HACS serves as closing-loop stabilizing substrate binding without catalytic function according to the literature (Zahn et al. J. Biol. Chem. 298(1) 101522 (2022)). Therefore, the c-terminal end was hypothesized to play an important role in limiting substrate size of the binding pocket. Based on the alanine scanning result on this region for both JGI15 and JG120, we can see the trend of decreasing overall activities especially under high formaldehyde concentration (5 mM) potentially due to decrease in the stability of the binding pocket. Interestingly, however, some mutations such as P547A (JGI15), P543A (JGI20), Q549A (JGI15) and Q544A (JGI20) show notable improvement in activity at low formaldehyde (0.5 mM) (FIG. 25B). This could be the result of altering substrate binding affinity with the smallest aldehyde, formaldehyde.

### Example 6: Improvement of HACS Activity by Creating Hybrid Protein of the Two High Performing Variants

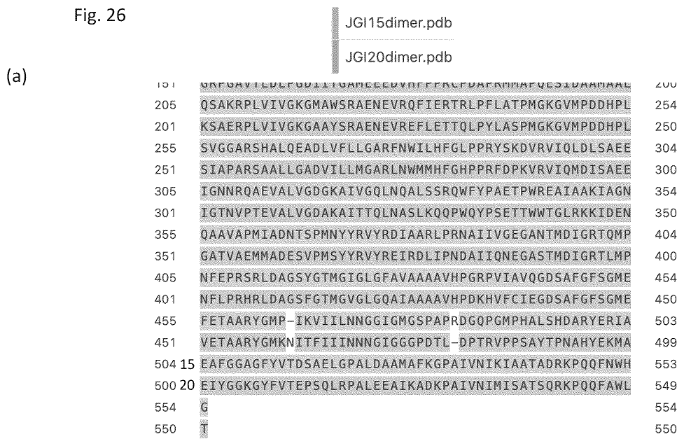

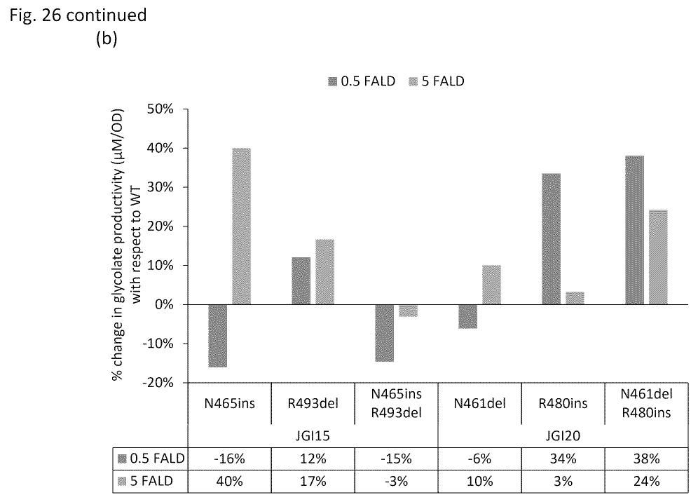

This example demonstrates the engineering of the recombinant high performing HACS variants (JGI15 and JGI20) by creating hybrid proteins based on structural analysis. Based on the kinetic characterization (Table 8), JGI20 has higher k_{cat} but also higher Kₘ with formaldehyde than JGI15. We hypothesized that we could improve either the affinity of JGI20 or the turnover of JGI15 by creating a hybrid protein between the two. To identify the structural difference between the two proteins we used “Pairwise Structure Alignment” function in the Protein Data Bank (PDB) website (www.rcsb.org). JG115 and JG120 structures modeled by AlphaFold were used for structure comparison and the result shows that there are two residues that are not aligned between the two protein structures (FIG. 26A). The JGI15-20 hybrid protein was constructed by inserting or deleting an AA residue to completely align the two structures. As a result, JGI15 N465ins, R493del and N465 R493del are constructed to make JGI15 “JGI20-like” whereas JGI20 N461del, R480ins and N461del R480ins are constructed to make JGI20 “JGI15-like” (FIG. 26B).

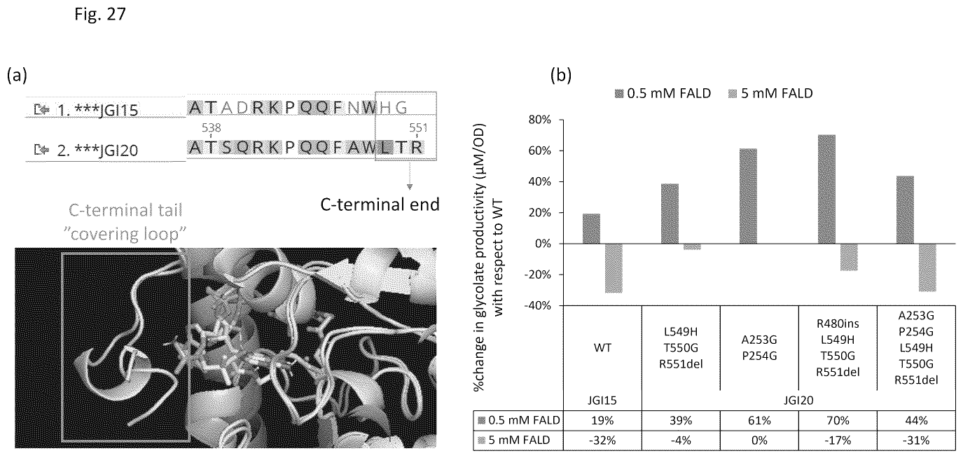

An alternative approach was based on improving the substrate binding affinity (Kₘ) of JG120 by engineering the active site of JGI20 to mimic JGI15. Comparing the active site residues (TABLE 11) of the two enzymes, the only unconserved residues are A253 and P254 of which JGI15 has two consecutive glycine residues in the corresponding position. Consequently, a “JGI15-like” JGI20 A253G P254G was constructed. Another target region was the c-terminal end, where alanine scanning results show changes in activities. JGI15 and 20 have highly conserved sequences at the c-terminal tail, except for the last four to five residues (FIG. 27A). Based on the protein structure of JGI15 and 20 modeled by AlphaFold, the difference in c-terminal end show slightly different orientation of the closing loop (FIG. 27A). We hypothesized that this difference could contribute to the difference in Kₘ between JGI15 and JGI20 and constructed “JGI15-like” c-terminal end of JGI20: JGI20 L549H T550G R551del.

TABLE 11
List of acyl-CoA kinases (ACK) and phosphoacyltransferases
(PTA) variants (JGIK) identified by selecting representative
genes from gene clusters with sequence similarity using
CcAck and CcPta as reference enzymes.

GenBank
Accession Number

| JGIK# | ACK | PTA |
|---|---|---|
| 1 | AKJ38693.1 | AKJ38694.1 |
| 2 | BCV24779.1 | BCV24778.1 |
| 3 | EDM85332.1 | EDM85331.1 |
| 4 | GFI65571.1 | GFI65570.1 |
| 5 | HBG22385.1 | HBG22386.1 |
| 6 | HFV10353.1 | HFV10352.1 |
| 7 | HGG13858.1 | HGG13857.1 |
| 8 | HIX51076.1 | HIX51075.1 |
| 9 | KXK65140.1 | KXK65141.1 |
| 10 | KXL51791.1 | KXL51790.1 |
| 11 | MBE5816841.1 | MBE5816840.1 |
| 12 | MBE6451999.1 | MBE6451998.1 |
| 13 | MBF0205569.1 | MBF0205570.1 |
| 14 | MBI4335478.1 | MBI4335477.1 |
| 15 | MBN1633386.1 | MBN1633385.1 |
| 16 | MBR2784446.1 | MBR2784445.1 |
| 17 | MBR3082232.1 | MBR3082233.1 |
| 18 | MBS5449595.1 | MBS5449596.1 |
| 19 | MBS6942200.1 | MBS6942201.1 |
| 20 | MBU0667180.1 | MBU0667181.1 |
| 21 | MBU2064158.1 | MBU2064159.1 |
| 22 | MBU2501086.1 | MBU2501087.1 |
| 23 | NLA96479.1 | NLA96478.1 |
| 24 | NLI62094.1 | NLI62095.1 |
| 25 | NLM93282.1 | NLM93283.1 |
| 26 | NMA59030.1 | NMA59029.1 |
| 27 | OGI05344.1 | OGI05345.1 |
| 28 | OHB58473.1 | OHB58472.1 |
| 29 | PWM39673.1 | PWM39672.1 |
| 30 | TKJ47541.1 | TKJ47542.1 |
| 31 | WP_022744670.1 | WP_022744669.1 |
| 32 | WP_023275423.1 | WP_023275424.1 |
| 33 | WP_076546120.1 | WP_076546119.1 |
| 34 | WP_078810629.1 | WP_078810628.1 |
| 35 | WP_099343330.1 | WP_099343331.1 |
| 36 | WP_106012460.1 | WP_106012461.1 |
| 37 | AAA72042.1 | AAA72041.1 |
| 38 | BAG33697.1 | BAG33698.1 |

The construction and testing of mutants are conducted in an identical format as what is described in EXAMPLE 5.

The JGI15-20 hybrid based on structure alignment shows notable improvement of JG115 at high formaldehyde and JG120 at low formaldehyde, which interestingly exhibits positive impact in both variants (FIG. 26B). It is possible that aligning the two variants affect the orientation of catalytic residues that changes the activity specifically under high or low formaldehyde concentration. Mutagenesis on JG120 active site and c-terminal end to mimic JGI15 showed more significant improvement in glycolate productivity of 39% and 61% respectively, under low formaldehyde concentration (FIG. 27B). We have also tried combining beneficial mutations from JG115-20 structure hybrid and active site hybrid and JGI20 R480 L549H T550G R551del combining AlphaFold structure hybrid and c-terminal end hybrid showed the best improvement in activity of up to 70% with only minimal drop in the activity at high formaldehyde concentration (FIG. 27B). This is close to 50% improvement from JG115 at 0.5 mM FALD which indicates notable reduction in the Kₘ of JGI20, which is intended from the hybrid approach.

### Example 7: Identification, Synthesis and Screening of Second Round HACS Variants for Activities with Formaldehyde

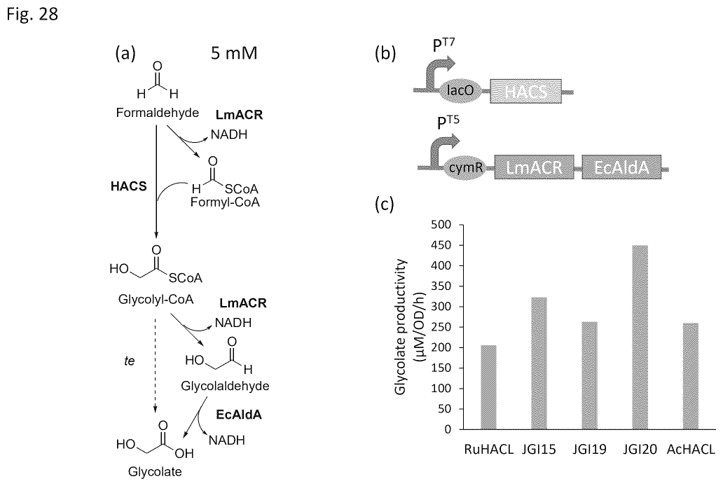

This example demonstrates the identification, synthesis, and screening of the second round HACS variants with formaldehyde as substrate. From the first-round variants, we found JG115, JG119 and JG120 to be active for glycolyl-CoA synthase activity exceeding the starting reference enzyme, RuHACL (FIG. 28C). Also, potential glycolyl-CoA synthase (C1-C1 condensation) activity of 2-hydroxyacyl-CoA lyase from Actinomycetospora chiangmaiensis (AcHACL) was demonstrated in literature (Rohwerder et al. Front. Microbiol. 11:691 (2020)). Based on in vivo screening using formaldehyde as sole carbon source (FIG. 28A) (EXAMPLE 3 pathway 1), AcHACL showed better glycolate productivity than RuHACL under 5 mM formaldehyde. AcHACL is distantly related to RuHACL and other first round JGI variants as seen from the phylogenetic tree (FIG. 29). Therefore, we decided to identify second round HACS variants (JGIH) using JG115, JG119, JG120 from first round variants and AcHACL as reference enzymes.

TABLE 6
List of 2-hydroxyacyl-CoA (HACS) variants (JGIH)
identified by selecting representative genes from
gene clusters with sequence similarity using AcHACL,
JGI15, JGI19 and JGI20 as reference enzymes.

| | GenBank |
|---|---|
| JGIH# | Accession Number |
| 1 | HIG47824.1 |
| 2 | TMD03111.1 |
| 3 | MBJ56818.1 |
| 4 | WP_095860310.1 |
| 5 | MBL8483477.1 |
| 6 | WP_058697592.1 |
| 7 | WP_130292058.1 |
| 8 | WP_207956071.1 |
| 9 | WP_132429652.1 |
| 10 | WP_060575023.1 |
| 11 | WP_068796145.1 |
| 12 | OJY48151.1 |
| 13 | WP_062397209.1 |
| 14 | WP_169186431.1 |
| 15 | WP_133828190.1 |
| 16 | MBS0560157.1 |
| 17 | PCJ59575.1 |
| 18 | MXY78649.1 |
| 19 | MBA01399.1 |
| 20 | MXX31676.1 |
| 21 | MXV80929.1 |
| 22 | MBI4083577.1 |
| 23 | MBK6319978.1 |
| 24 | MBI5948182.1 |
| 25 | PFG74273.1 |
| 26 | WP_158065972.1 |
| 27 | MBN9492325.1 |
| 28 | MBK6663287.1 |
| 29 | MBI2766664.1 |
| 30 | HEM18354.1 |
| 31 | GBD22648.1 |
| 32 | MBF6599205.1 |
| 33 | MXW00101.1 |
| 34 | MYA07641.1 |
| 35 | REJ76484.1 |
| 36 | HDY15625.1 |
| 37 | MBW2231087.1 |
| 38 | NRA08835.1 |
| 39 | NQZ98823.1 |
| 40 | MBI3918747.1 |
| 41 | MBI2761137.1 |
| 42 | MBE0608783.1 |
| 43 | MYA54281.1 |
| 44 | NRA01576.1 |
| 45 | MBW2623123.1 |
| 46 | MBI5615765.1 |
| 47 | MSR14309.1 |
| 48 | XP_004342722.2 |
| 49 | MSP42197.1 |
| 50 | TDI61101.1 |
| 51 | MBO0741576.1 |
| 52 | MBO0736096.1 |
| 53 | MBV9828771.1 |
| 54 | MAW55136.1 |
| 55 | MBV38827.1 |
| 56 | TMJ68231.1 |
| 57 | TMJ64557.1 |
| 58 | MBV9815528.1 |
| 59 | MYH41266.1 |
| 60 | MPZ97997.1 |
| 61 | MBT5774752.1 |
| 62 | XP_014714961.1 |
| 63 | TAK78428.1 |
| 64 | TAJ19927.1 |
| 65 | PKN81274.1 |
| 66 | RLT34960.1 |
| 67 | MBT5775398.1 |
| 68 | TMD99851.1 |
| 69 | MSQ12864.1 |
| 70 | MBL0714078.1 |
| 71 | WP_114297888.1 |
| 72 | MAK25262.1 |
| 73 | WP_068138361.1 |
| 74 | RMG94145.1 |
| 75 | MBA4180234.1 |
| 76 | MBM3723043.1 |
| 77 | ABF11225.1 |
| 78 | TAL98798.1 |
| 79 | NNN20496.1 |
| 80 | MBP1761901.1 |
| 81 | PPQ43247.1 |
| 82 | MSQ25793.1 |
| 83 | TMK28344.1 |
| 84 | HIB12002.1 |
| 85 | WP_179589464.1 |
| 86 | MXY42918.1 |
| 87 | WP_184156128.1 |
| 88 | HET53513.1 |
| 89 | TMK22624.1 |
| 90 | MXX66290.1 |
| 91 | GIS94895.1 |
| 92 | MBN1557905.1 |
| 93 | MSV30368.1 |
| 94 | MBN2179295.1 |
| 95 | TDI90456.1 |
| 96 | OGN76415.1 |
| 97 | WP_102074055.1 |
| 98 | PZC47999.1 |
| 99 | HHH88785.1 |
| 100 | OLB93949.1 |
| 101 | PKB76696.1 |
| 102 | HED24197.1 |
| 103 | WP_066960443.1 |
| 104 | WP_169259343.1 |
| 105 | WP_201494572.1 |
| 106 | MBN9621549.1 |
| 107 | OZG26106.1 |
| 108 | WP_016501746.1 |

Method described in EXAMPLE 1 (FIG. 19) was used for each of the starting reference enzyme. Total 99 enzymes are identified that are closely related to AcHACL (AcHACL cluster), distantly related to AcHACL (distantly related to AcHACL cluster), JGI19 cluster, JGI15 cluster and JGI20 cluster (FIG. 29). We have also identified 9 extra enzymes from I-TASSER (Yang et al. Nature Methods, 12:7-8 (2015)) that are structurally similar to AcHACL, JGI15, JGI19 or JGI20 without considering the sequence similarity. Total 108 genes (JGIH1 to JGIH108) are codon-optimized and synthesized in collaboration with Joint Genome Institute and 99 variants are successfully constructed to pCDFDuet-1 expression vector for testing.

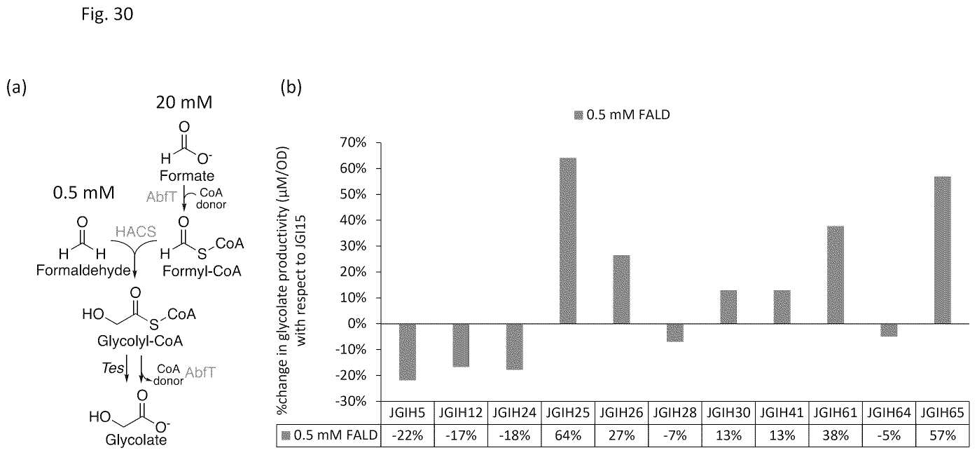

The second-round variants are tested for glycolyl-CoA synthase activity using the high throughput screening co-feeding formaldehyde (0.5 mM) and formate with formate activation enzyme (FIG. 30A). The result shows that six variants (JGIH25, 26, 30, 41, 61 and 65) perform better than wildtype JGI15, with JGIH25 and 65 exceeding 50% increase in glycolate productivity. There are 5 additional candidates that perform at similar level as JGI15 (FIG. 30B).

Based on the phylogenetic tree analysis, JGIH25, 26 and 30 belong to the JGI20 cluster, 41 and 61 belong to the JGI15 cluster and JGI65 belongs to the JGI19 cluster. A couple variants from AcHACL cluster (JGIH5 and 12) also show decent glycolate productivity at approximately 80% of JGI15. When comparing the residues of the six best variants aligned to the active site residues of JGI20 identified from EXAMPLE 5, we can see most of them are highly conserved with exception of the two residues (A253 P254 of JGI20) that were not conserved between JGI15 and JGI20 (TABLE 7). JGIH61 and 65 are the most phylogenetically distant from JGI15 and 20 and hence, there are multiple unconserved residues at the active site other than the two previously identified. After further characterization of the two variants such as their affinity with formaldehyde and formyl-CoA and turnover rate, constructing new hybrid proteins based on the learnings from previous JG115 and 20 hybrid approach could be considered. The c-terminal residues of six best variants are also well-conserved except that JGIH61 and 65 have two and three extra AA residues at the c-terminal end. None of the six variants have the same c-terminal end residues as JG115, which could be another target for hybrid protein approach for the closing loop.

### Example 8: Screening of First and Second Round Variants for Activities with Aldehydes

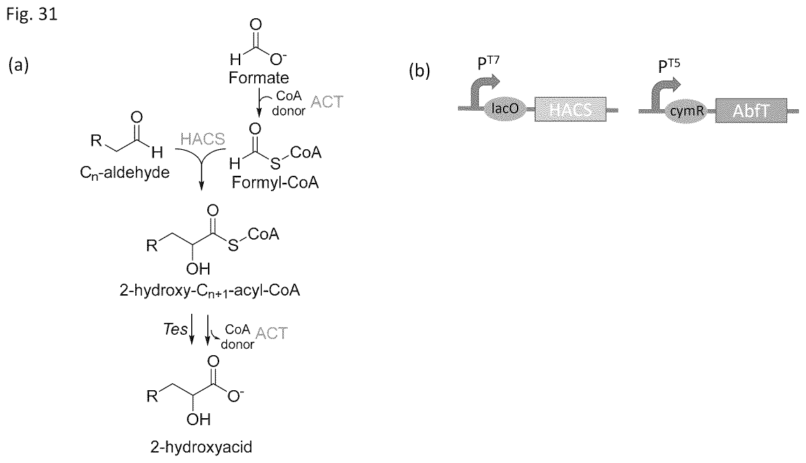

The purpose of this example is to demonstrate high throughput platform for screening first and second round of 2-hydroxyacyl-CoA synthase (HACS) variants with various aldehydes as the substrate in vivo. We used 2-hydroxyacid productivity per cell density (μM/OD600) as indicator of HACS activity. 2-Hydroxyacids can be produced co-feeding various aldehydes and formic acid (formate) as carbon source in the presence of active HACS variant and acyl-CoA transferase from Clostridium aminobutyricum (CaAbfT). CaAbfT is shown to be capable of catalyzing reaction from formate to formyl-CoA (Nattermann, M., et al. ACS Catal 11 (9): 5396-5404 (2021)). HACS condenses aldehyde and formyl-CoA to form 2-hydroxyacyl-CoA, which can then be hydrolyzed to 2-hydroxyacid via native thioesterase activities (FIG. 31A).

For the in vivo prototyping, we engineered vectors to independently control expression of HACS variants and CaAbfT, with HACS under control of the IPTG-inducible T7 promoter in pCDFDuet-1 and CaAbfT under control of a cumate-inducible T5 promoter in pETDuet-1 (FIG. 31B) and transformed them into the engineered strain of E. coli described in EXAMPLE 3.

The HACS variants were screened for 2-hydroxyacid production using the high throughput screening platform as described in EXAMPLE 3 by co-feeding 5 mM aldehyde and 20 mM formate. The cells were harvested after 1 hour by centrifugation and the supernatant analyzed by HPLC (as described in EXAMPLE 2) or SoGO method.

In the SoGO method, glycolate oxidase from Spinacia oleracea (SoGO) is used to catalyze oxidation of 2-hydroxyacid to produce 2-oxoacid and hydrogen peroxide (H₂O₂). Then, Amplex UltraRed (Invitrogen) reagent is used as a fluorogenic substrate for horseradish peroxidase (HRP) (Sigma) that reacts with H₂O₂ in a 1:1 stoichiometric ratio to produce Amplex UltroxRed, a brightly fluorescent and strongly absorbing reaction product (excitation/emission maxima ˜568/581 nm). 2-hydroxyacid concentration was calculated based on the calibration of the fluorescent reading measured by Amplex UltroxRed using a BioTek Synergy HT plate reader (BioTek Instruments).

### Example 9: Screening of First and Second Round HACS Variants for Activity with Acetaldehyde

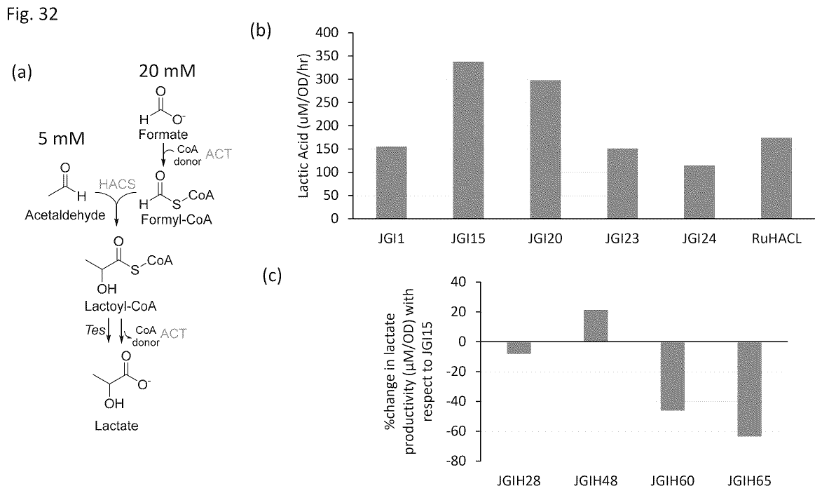

This example demonstrates the screening of the first and second round HACS variants with acetaldehyde as the substrate in vivo. We used lactic acid (lactate) productivity per cell density (uM lactate/OD600) as indicator of HACS activity. The HACS variants were screened for lactate production using the high throughput screening platform as described in EXAMPLE 3 by co-feeding 5 mM acetaldehyde and 20 mM formate. HACS condenses acetaldehyde and formyl-CoA to form lactoyl-CoA, which can then be hydrolyzed to lactate via native thioesterase activities (FIG. 32A).

The screening of first round HACS variants shows that six variants out of 29 giving decent lactate productivity (FIG. 32B). JGI15 and JG120 are the two best candidates showing more than 2-fold lactate productivity compared to other HACS variants.

Quantification of product concentration (lactate) for the second round HACS variants were determined via SoGO method as described in EXAMPLE 8. The results show that one variant JGIH48 perform better than wildtype JGI15, with exceeding 20% increase in lactate productivity (FIG. 32C). Moreover, JGIH28 performs at similar level as JGI15.

### Example 10: Screening of First and Second Round HACS Variants for Activity with Propionaldehyde

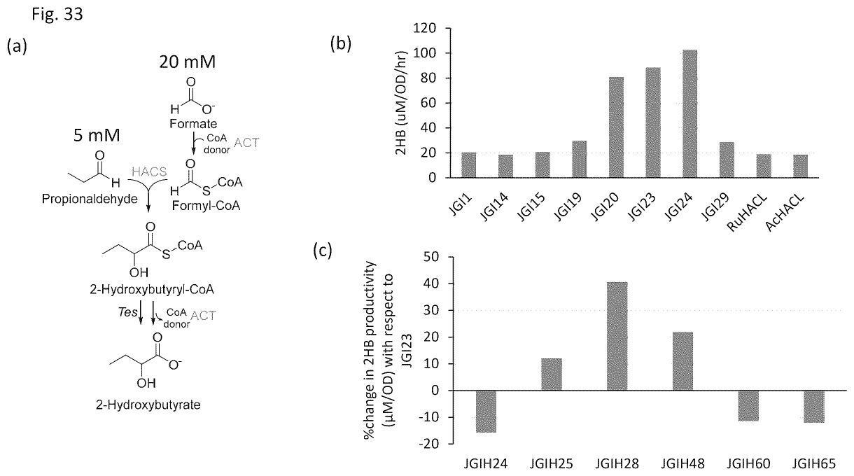

This example demonstrates the screening of the first and second round HACS variants with propionaldehyde as the substrate in vivo. We used 2-hydroxybutyric acid (2HB) productivity per cell density (μM 2HB/OD600) as indicator of HACS activity. The HACS variants were screened for 2HB production using the high throughput screening platform as described in EXAMPLE 3 by co-feeding 5 mM propionaldehyde and 20 mM formate. HACS condenses propionaldehyde and formyl-CoA to form 2-hydroxybutyryl-CoA, which can then be hydrolyzed to 2HB via native thioesterase activities (FIG. 33A).

The screening of first round HACS variants shows that ten variants out of 29 giving decent 2HB productivity (FIG. 33B). JG120, JGI23 and JG124 are the three best candidates showing more than 3-fold 2HB productivity compared to other HACS variants.

Quantification of product concentration (2HB) for the second round HACS variants were determined via SoGO method as described in EXAMPLE 8. The results show that three variants (JGIH25, JGIH28 and JGIH48) perform better than JG123, with JGIH28 exceeding 40% increase in 2HB productivity (FIG. 33C).

### Example 11: Screening of First and Second Round HACS Variants for Activity with Glycolaldehyde

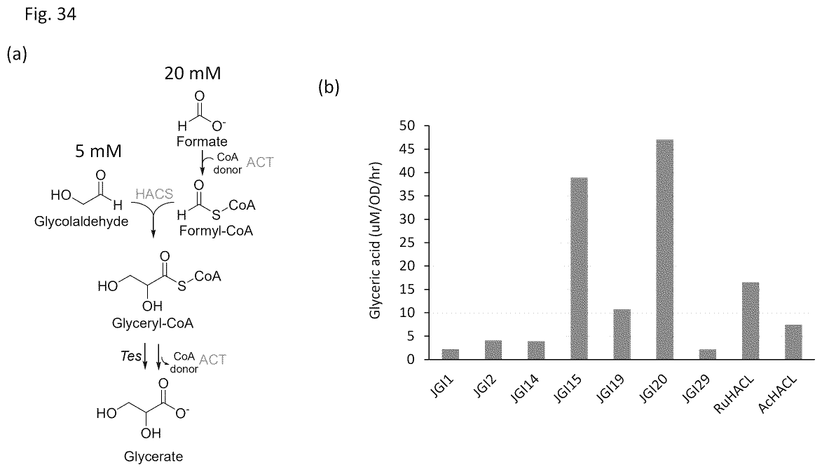

This example demonstrates the screening of the first and second round HACS variants with glycolaldehyde as the substrate in vivo. We used glyceric acid (glycerate) productivity per cell density (uM glycerate/OD600) as indicator of HACS activity. The HACS variants were screened for 2HB production using the high throughput screening platform as described in EXAMPLE 3 by co-feeding 5 mM glycolaldehyde and 20 mM formate. HACS condenses glycolaldehyde and formyl-CoA to form glyceryl-CoA, which can then be hydrolyzed to glycerate via native thioesterase activities (FIG. 34A).

The screening of first round HACS variants shows that nine variants out of 29 giving decent glycerate productivity (FIG. 34B). JGI15 and JGI20 are the two best candidates showing more than 2-fold glycerate productivity compared to other HACS variants.

Based on the phylogenetic tree analysis (FIG. 29), since JGI15 and JGI20 are the best performing candidates from the first round HACS screening, we expect the variants that belong to the JGI15 and/or JGI20 clusters show decent performance (glycerate productivity) for the second round HACS variants screening.

### Example 12: Screening of First and Second Round HACS Variants for Activity with Glyoxylic Acid

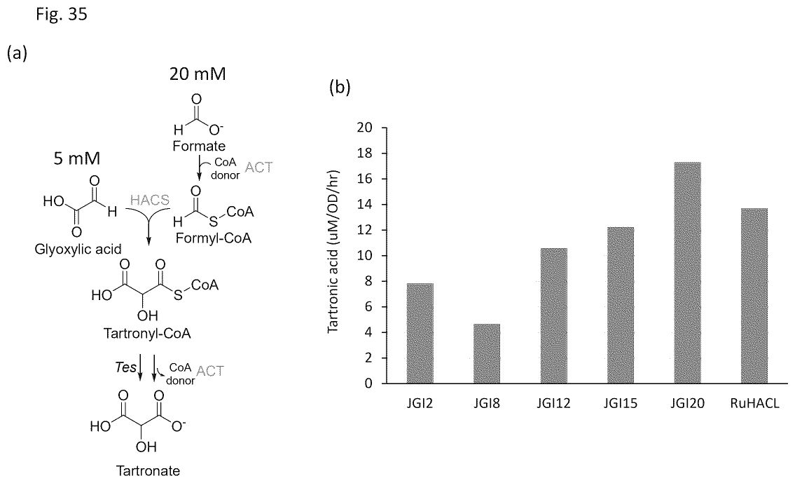

This example demonstrates the screening of the first and second round HACS variants with glyoxylic acid (glyoxylate) as the substrate in vivo. We used tartronic acid (tartronate) productivity per cell density (uM tartronate/OD600) as indicator of HACS activity. The HACS variants were screened for tartronate production using the high throughput screening platform as described in EXAMPLE 3 by co-feeding 5 mM glyoxylate and 20 mM formate. HACS condenses glyoxylate and formyl-CoA to form tartronyl-CoA, which can then be hydrolyzed to tartronate via native thioesterase activities (FIG. 35A).

The screening of first round HACS variants shows that six variants out of 29 giving decent tartronate productivity (FIG. 35B). JGI20 is the best candidate showing 30% better tartronate productivity compared to other HACS variants.

Based on the phylogenetic tree analysis (FIG. 29), since JGI20 is the best performing candidate from the first round HACS screening, we expect the variants that belong to the JGI20 cluster show decent performance (tartronate productivity) for the second round HACS variants screening.

### Example 13: Screening of First and Second Round HACS Variants for Activity with 3-Hydroxypropionaldehyde

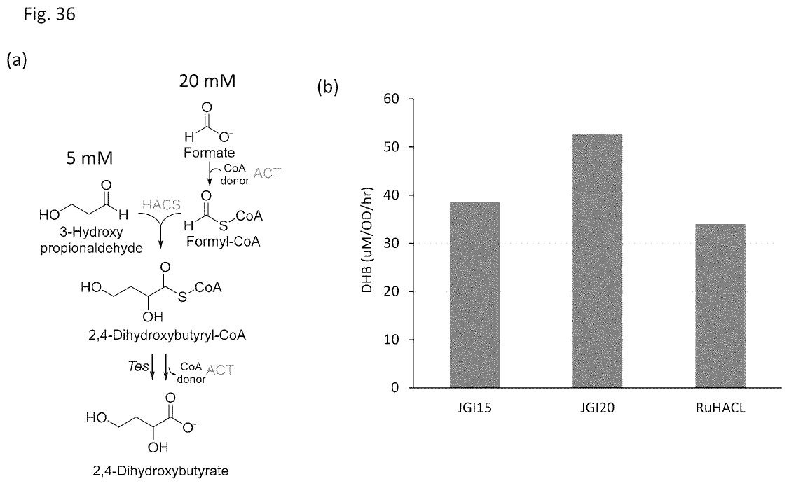

This example demonstrates the screening of the first and second round HACS variants with 3-hydroxypropionaldehyde (3HP) as the substrate in vivo. We used 2,4-dihydroxybutyric acid (DHB) productivity per cell density (uM DHB/OD600) as indicator of HACS activity. The HACS variants were screened for DHB production using the high throughput screening platform as described in EXAMPLE 3 by co-feeding 5 mM 3HP and 20 mM formate. HACS condenses 3HP and formyl-CoA to form 2,4-dihydroxybutyryl-CoA, which can then be hydrolyzed to DHB via native thioesterase activities (FIG. 36A).

The screening of first round HACS variants shows that three variants out of 29 (JGI15, JG120 and RuHACL) giving decent DHB productivity (FIG. 36B).

Based on the phylogenetic tree analysis (FIG. 29), since JG115, JG120 and RuHACL are the best performing candidates from the first round HACS screening, we expect the variants that belong to these clusters show decent performance (DHB productivity) for the second round HACS variants screening.

### Example 14: Screening of First Round Variants for Activities with Ketones

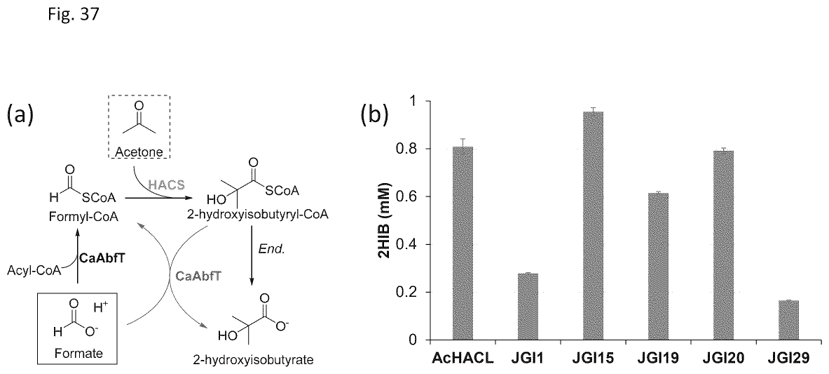

This example demonstrates screening of the first second round HACS variants with various ketones as substrates for branched-chain compounds production. The HACS variants are tested using the high throughput screening platform as described in EXAMPLE 3 pathway 2 by co-feeding 100 mM acetone and 20 mM formate with formate activation enzyme CaAbfT (FIG. 37A).

The result shows that JG115, JG119, and JGI20 together AcHACL have better performance than other HACLs, and JGI15 has the best performance. Kinetic characterization of JG115 and AcHACL with acetone and formate were performed using the method described in EXAMPLE 4. JGI15 has much better activity (higher K_{cat}) which gives better performance in vivo, while it has much higher Kₘ which limited its performance (Table 8). Although AcHACL has worse activity, it has much lower Kₘ (FIG. 37B). Better HACL for 2HIB production by condensation of acetone and formyl-CoA are expected in the second round HACS variants using AcHACL or JG115 as the reference.

### Example 15: Methyl Ketones as Substrate for Condensation with Formyl-Coa Via In Vitro Assays

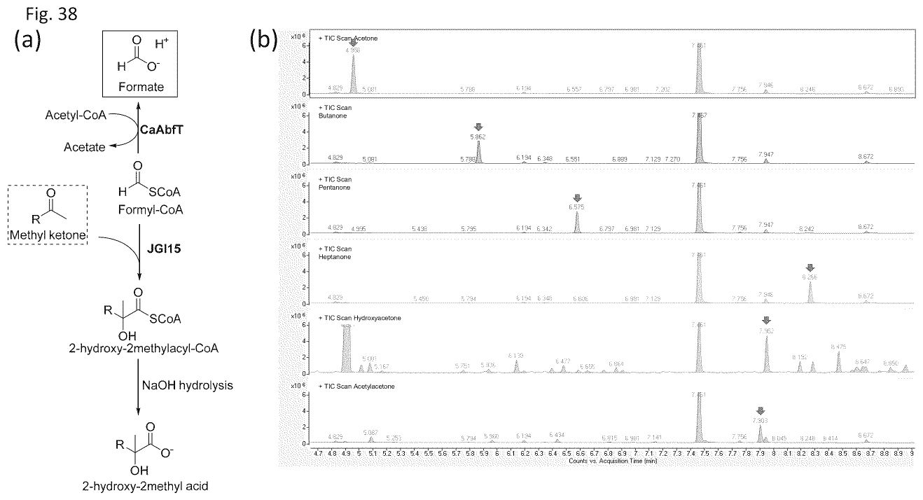

This example demonstrates the implementation of the condensation of methyl ketone with formyl-CoA using purified enzymes. The formyl-CoA generation catalyzed by CoA transferase CaAbfT and condensation catalyzed by HACS JGI15 is identical to the examples described above (FIG. 38A). Methyl ketones can be produced through fatty acids synthesis and β-oxidation pathway demonstrated in literatures (Goh E-B, et al., Appl Environ Microbiol 78:70-80(2012); Nies S C, et al., Metab Eng 62:84-94 (2020)).

The enzymes CoA transferase CaAbfT and HACS JG115 were overexpressed and purified as described above. In vitro purified enzyme reactions for condensation of methyl ketone and formyl-CoA were comprised of 100 mM KPi pH 6.9, 10 mM MgCl2, 0.15 mM TPP, 2 mM acetyl-CoA, 1 μM JGI15, 2 μM CaAbfT, 20 mM formate and 100 mM tested methyl ketones. Reactions were incubated at 30° C. for 24 hours unless otherwise specified.

For this analysis, samples containing acyl-CoAs were first treated with 1/20 of the reaction volume of 10 M NaOH solution was added to terminate the reactions. After 30 min hydrolysis, 1/20 of the reaction volume of 10 N H₂SO₄ was added to improve the efficiency of acid extraction. The resulting sample was extracted into 4 mL ethyl acetate by vigorous vortexing for 90 seconds. The organic phase was separated and evaporated to dryness under a stream of nitrogen. The residue was dissolved in 50 μL pyridine and 50 μL N,O-Bis(trimethylsilyl)trifluoroacetamide (BSTFA) and incubated at 60° C. for 15 minutes. Compound identification and analysis was performed by GC-MS using an Agilent 7890B Series Custom Gas Chromatography system equipped with a 5977B Inert Plus Mass Selective Detector Turbo EI Bundle (for identification) and an Agilent HP-5-ms capillary column (0.25 mm internal diameter, 0.25 μm film thickness, 30 m length). Samples were analyzed by GC (1 μL injection with a 20:1 split ratio) using helium as the carrier gas at a flowrate of 1.5 mL/min and the following temperature profile: initial 90° C. for 3 min; ramp at 15° C./min to 170° C.; ramp at 20° C./min to 300° C. and hold for 8 min. The injector and detector temperature were 250° C. and 350° C., respectively.

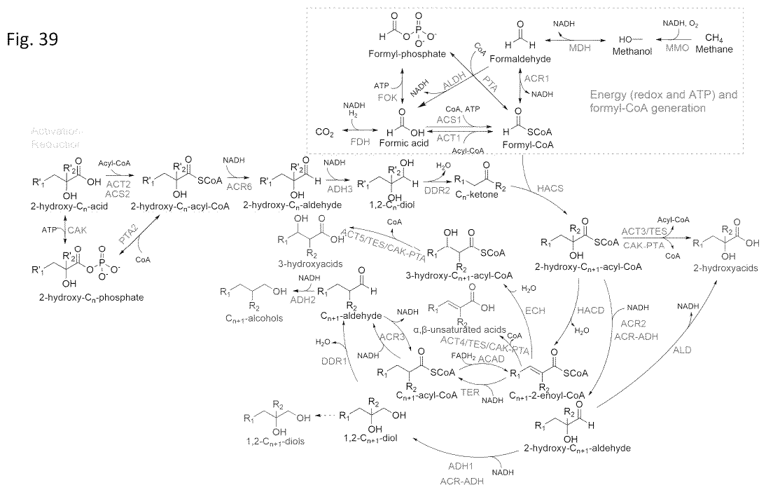

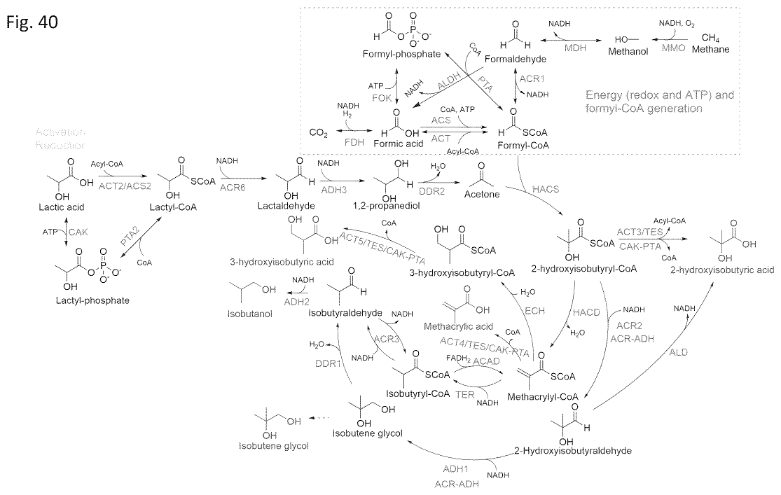

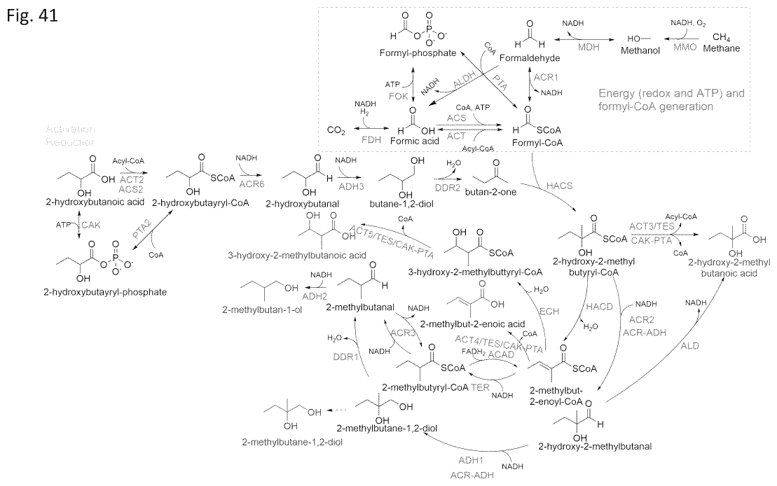

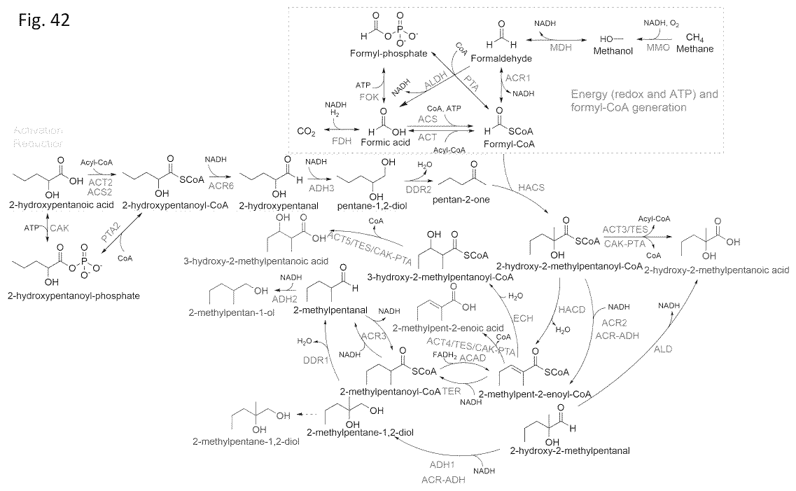

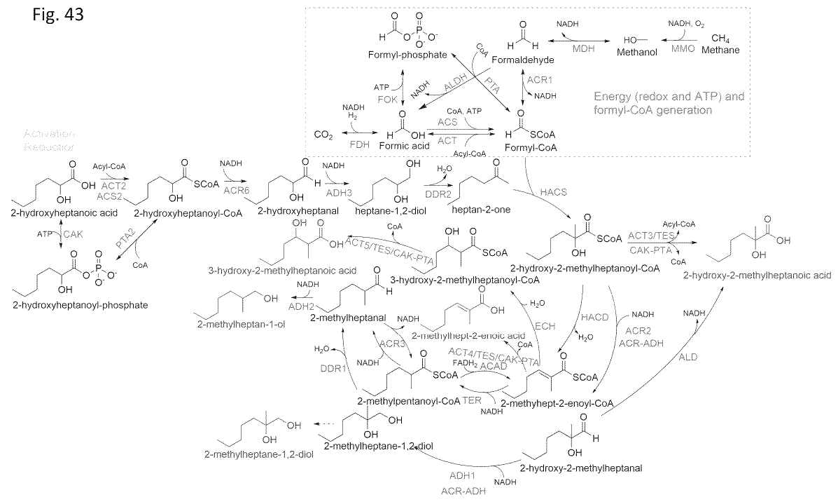

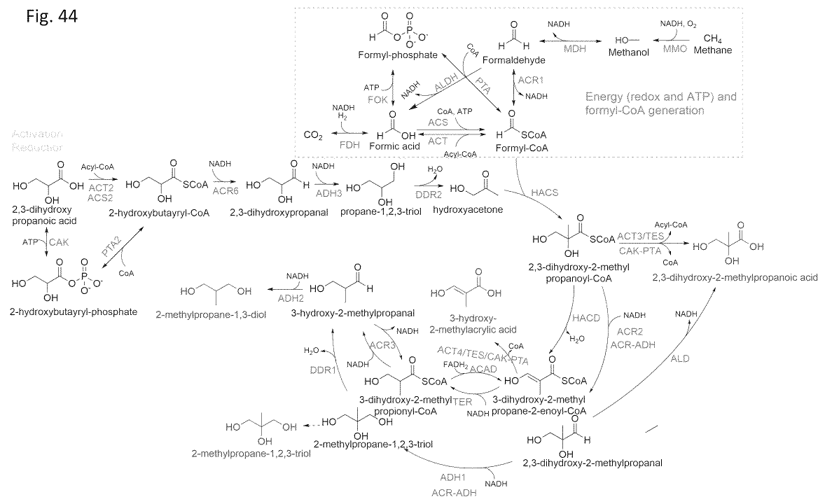

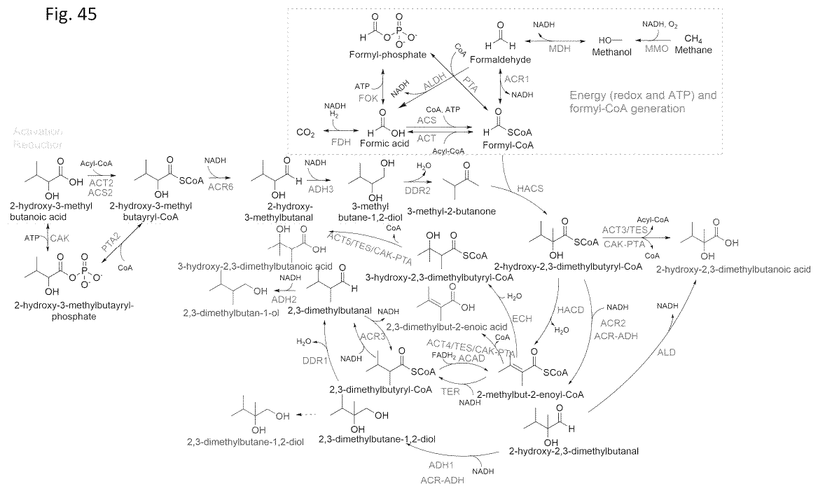

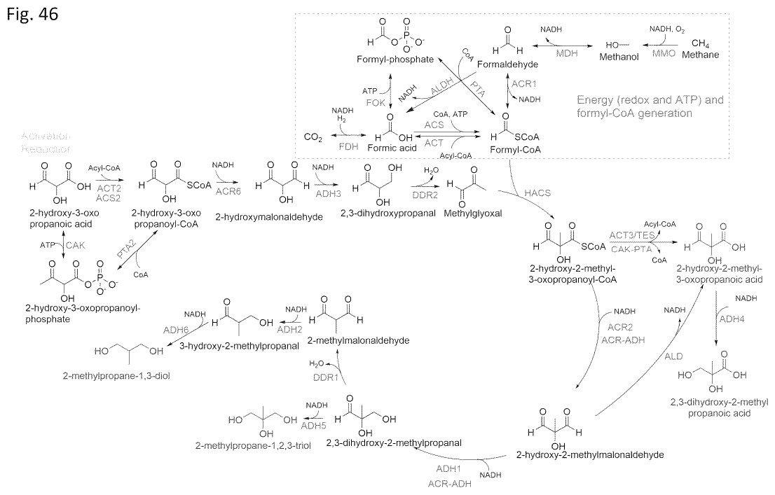

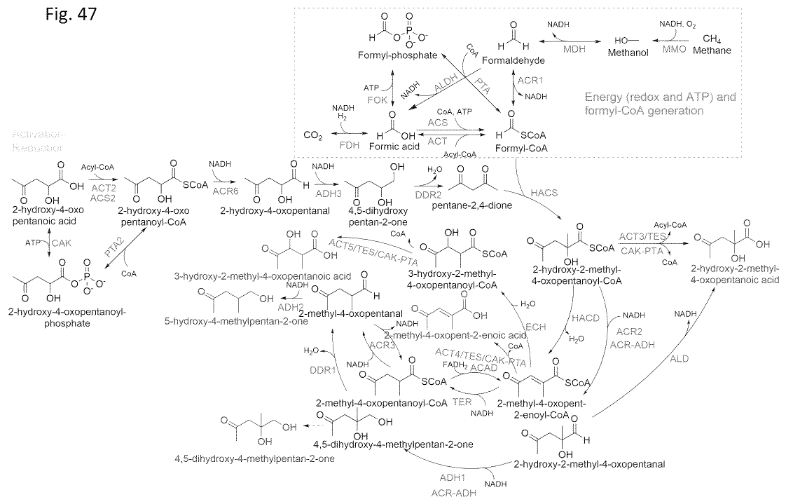

The methyl ketones can be used for condensation including but not limited to acetone, methyl ethyl ketone (Cn-ketone, n>3, butanone, pentanone and heptanone as example), Hydroxylated ketones (hydroxyacetone), and other functional ketones (acetylacetone, branched-chain ketones, methylglyoxal) etc. The JGI15 could catalyze the condensation of tested ketones as shown in FIG. 38B, which indicates the other identified HACS is able to condensation of other ketones with formyl-CoA to produce 2-hydroxy-2 methyl acid and derivatives (FIG. 39). The methyl ketones can be used for condensation including but not limited to acetone, methyl ethyl ketone (Cn-ketone, n>3, butanone, pentanone and heptanone as examples), Hydroxylated ketones (hydroxyacetone), and other functional ketones (acetylacetone, branched-chain ketones, methylglyoxal) (FIG. 40-47).

### Example 16: Identification, Synthesis and Screening of ACR Variants

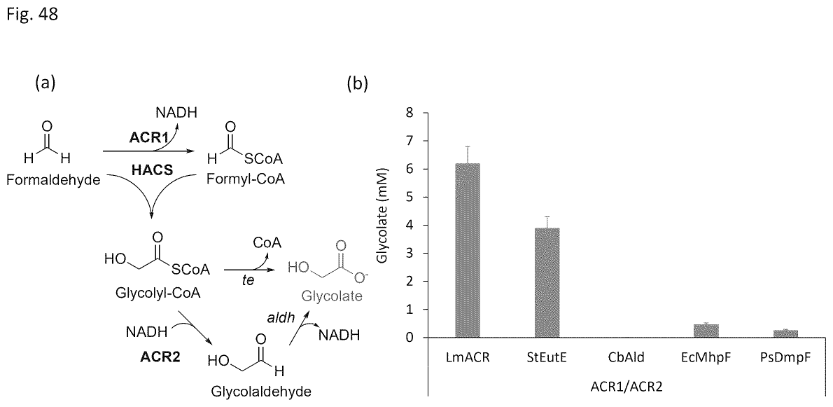

This example demonstrates the identification, synthesis, and screening of the acyl-CoA reductase (ACR) variants specifically for acylating formaldehyde oxidation (formaldehyde to formyl-CoA) reaction. From initial screening of known ACRs measured by glycolate productivity coupled with RuHACL (FIG. 48A), we identified acyl-CoA reductase from Listeria monocytogenes (LmACR) to be the most active (FIG. 48B), which was chosen as the starting reference for identifying new enzymes with sequence similarity.

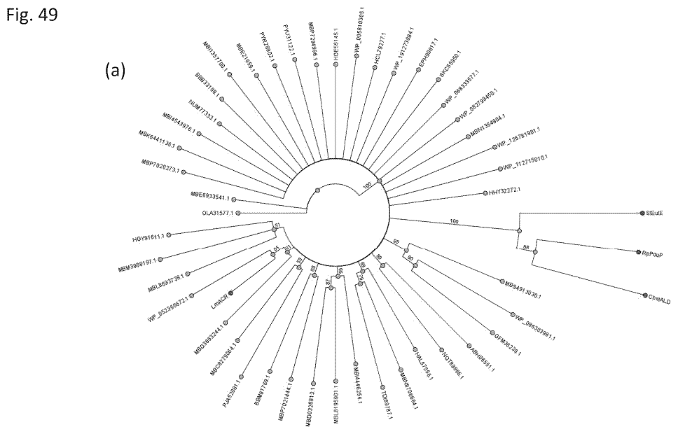

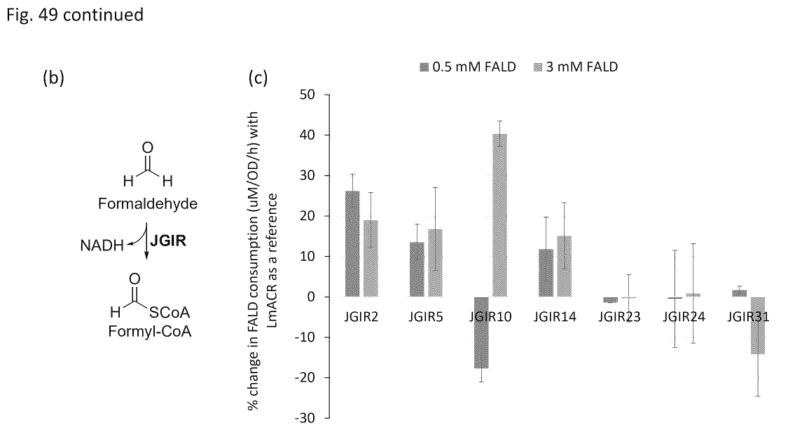

Using the method described in EXAMPLE 1 (FIG. 19), 44 new ACR variants (JGIR1 to 44) are identified, and 40 final constructs are synthesized in collaboration with Joint Genome Institute (FIG. 49A, TABLE 9).

TABLE 9
List of acyl-CoA reductase (ACR) variants (JGIR) identified
by selecting representative genes from gene clusters with
sequence similarity using LmACR as reference enzyme.

| | GenBank |
|---|---|
| JGIR# | Accession Number |
| 1 | ABX41556.1 |
| 2 | WP_185879480.1 |
| 3 | WP_051457024.1 |
| 4 | WP_088269124.1 |
| 5 | WP_087641473.1 |
| 6 | WP_051541705.1 |
| 7 | HHY51863.1 |
| 8 | WP_119112248.1 |
| 9 | WP_114642697.1 |
| 10 | WP_202656015.1 |
| 11 | WP_115130814.1 |
| 12 | WP_052127368.1 |
| 13 | MBN6206692.1 |
| 14 | WP_051217603.1 |
| 15 | WP_129009770.1 |
| 16 | MTK10086.1 |
| 17 | WP_106009325.1 |
| 18 | HBB29922.1 |
| 19 | WP_216437565.1 |
| 20 | WP_090039956.1 |
| 21 | WP_083963349.1 |
| 22 | WP_012101452.1 |
| 23 | WP_122646076.1 |
| 24 | WP_070791575.1 |
| 25 | WP_010715224.1 |
| 26 | WP_094899923.1 |
| 27 | WP_152887945.1 |
| 28 | HAR84250.1 |
| 29 | MBS5083929.1 |
| 30 | WP_027296492.1 |
| 31 | WP_104149274.1 |
| 32 | WP_135035016.1 |
| 33 | EJO19495.1 |
| 34 | WP_051245790.1 |
| 35 | WP_125552613.1 |
| 36 | HCL03003.1 |
| 37 | WP_126792036.1 |
| 38 | NQJ18683.1 |
| 39 | WP_191507870.1 |
| 40 | MBR5981728.1 |
| 41 | WP_215633330.1 |
| 42 | MBP3327663.1 |
| 43 | WP_152889336.1 |
| 44 | WP_052356672.1 |

The ACR variants are tested in the resting cell format identical to what is described in EXAMPLE 2 but without the presence of HACS to reduce complexity in the overall reaction scheme (FIG. 49B). As a result, formaldehyde reduction to formyl-CoA in vivo catalyzed by ACR variants was measured using high throughput NASH colorimetric assay (Nash, Biochem J. 55 (3): 416-421 (1953)). The formaldehyde consumption per cell density (OD600) was calculated and compared with LmACR activity as a reference (FIG. 49C). There are three variants (JGIR2, 5 and 14) that shows 10-20% improved formaldehyde consumption activities under both low formaldehyde (0.5 mM) and high formaldehyde (3 mM). JGIR10 shows significant improvement (40%) in activity under high formaldehyde but reduced activity at low formaldehyde in comparison with LmACR, possibly indicating higher k_{cat} yet higher Kₘ as well. There are several other variants that have comparable activities with LmACR which are worth further exploring in other conditions.

### Example 17: Identification, Synthesis and Screening of Formate Activation Enzyme (Act and Ack-PTA) Variants

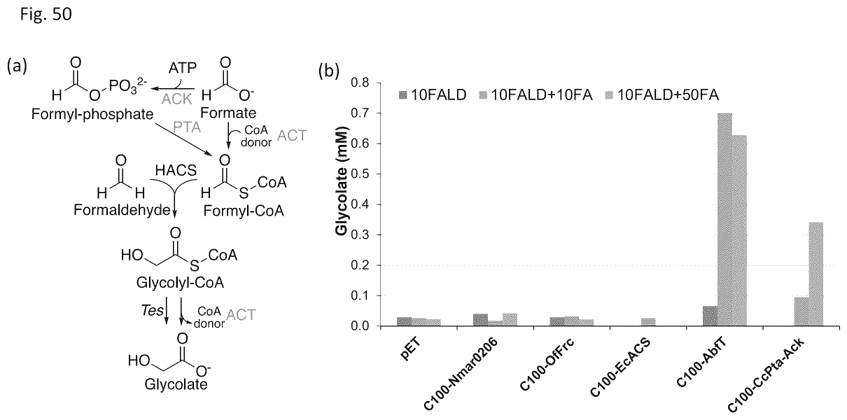

This example demonstrates the identification, synthesis and screening of the acyl-CoA transferase (ACT) variants and acyl-CoA kinase (ACK) and phosphoacyltransferase (PTA) variants specifically for formate activation (formate to formyl-CoA) reaction (FIG. 50A). From initial screening of known ACTs and ACK-PTAs measured by glycolate productivity coupled with JGI15 (FIG. 50B), we identified acyl-CoA transferase from Clostridium aminobutyricum (CaAbfT) to be the most active from ACT variants and acyl-CoA kinase and phosphoacyltransferase combination from Clostridium cylindrosporum (CcAck-Pta) to be the most active from ACK-PTA variants (FIG. 50B), which were chosen as the starting reference for identifying new enzymes with sequence similarity.

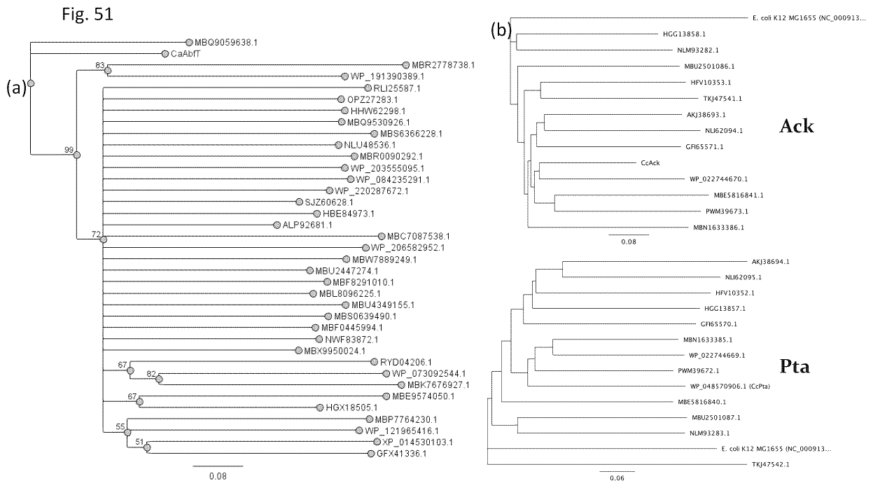

Using the method described in EXAMPLE 1 (FIG. 19), 62 new ACT variants (JGIT1 to 62) and 38 new ACK-PTA variants are identified are synthesized in collaboration with Joint Genome Institute (FIG. 51A, FIG. 51B, TABLE 10).

TABLE 10
List of acyl-CoA transferases (ACT) variants (JGIT) identified
by selecting representative genes from gene clusters with sequence
similarity using CaAbfT and OfFrc as reference enzymes.

| | GenBank Accession |
|---|---|
| JGIT# | Number |
| 1 | NBW24427.1 |
| 2 | HBE84973.1 |
| 3 | WP_132821656.1 |
| 4 | WP_220287672.1 |
| 5 | WP_121965416.1 |
| 6 | XP_014530103.1 |
| 7 | 2OAS_1 |
| 8 | WP_084235291.1 |
| 9 | MBS6366228.1 |
| 10 | MBR5999861.1 |
| 11 | 3D3U_1 |
| 12 | WP_073092544.1 |
| 13 | MBR0090292.1 |
| 14 | MBU4349155.1 |
| 15 | NLU48536.1 |
| 16 | 3EH7_1\|Chain |
| 17 | MBS0639490.1 |
| 18 | WP_194298948.1 |
| 19 | 3UBM_A |
| 20 | WP_203555095.1 |
| 21 | MBC7087538.1 |
| 22 | WP_191390389.1 |
| 23 | MBR0127170.1 |
| 24 | WP_206582952.1 |
| 25 | MBK5252043.1 |
| 26 | MBR2778738.1 |
| 27 | MBF8291010.1 |
| 28 | RLI25587.1 |
| 29 | HHW62298.1 |
| 30 | NWF83872.1 |
| 31 | NUN70050.1 |
| 32 | MBU2447274.1 |
| 33 | MBW7889249.1 |
| 34 | MBK7676927.1 |
| 35 | MBV8105822.1 |
| 36 | MBP7764230.1 |
| 37 | MSO92582.1 |
| 38 | MBE9574050.1 |
| 39 | MBX9950024.1 |
| 40 | AEK61848.1 |
| 41 | MBL8096225.1 |
| 42 | OPZ27283.1 |
| 43 | RYD04206.1 |
| 44 | MBQ9059638.1 |
| 45 | 2G39_1 |
| 46 | 2NVV_1 |
| 47 | MBQ9530926.1 |
| 48 | 4EU3_1 |
| 49 | KAF4531260.1 |
| 50 | 2HJ0_1 |
| 51 | SJZ60628.1 |
| 52 | ALP92681.1 |
| 53 | GFX41336.1 |
| 54 | 5VIT_1 |
| 55 | SDR52074.1 |
| 56 | MBF0160244.1 |
| 57 | MBF0445994.1 |
| 58 | HGX18505.1 |
| 59 | MBK9387745.1 |
| 60 | MBF0310027.1 |
| 61 | RKY20198.1 |
| 62 | NYH33089.1 |

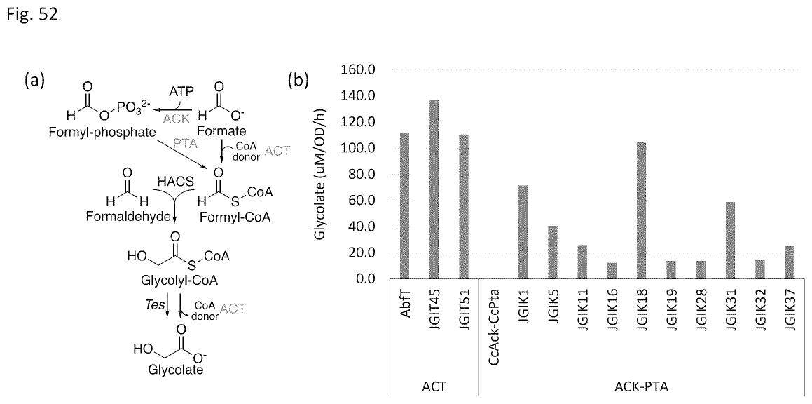

The ACT and ACK-PTA variants are tested in the resting cell format identical to what is described in EXAMPLE 3 (pathway 2) with JGI20 as HACS and different formate activation enzyme variants in the place of CaAbfT (FIG. 52A). 2.5 mM formaldehyde and 20 mM formate were added as carbon sources to measure glycolate activities of the variants. The result shows that two ACT variants (JGIRT45 and 51) show equal or improved CoA transferase activity and multiple ACK-PTA variants show notable formyl-CoA generation activity which was not observed from CcAck-Pta (FIG. 52B). It is consistent with our previous observation that CcAck-Pta requires high formate concentration (50 mM) to show notable glycolate productivity indicating high Km of this enzyme (FIG. 52B). Some ACK-PTA variants such as JGIK1, 18 and 31 show comparable activity with high performing ACT variants which could provide more diverse route for formyl-CoA generation even under relative low formate concentrations (FIG. 52B).

### Example 18: Strategies to Engineer and Screen Enzymes with Improved Catalytic Efficiency

This example demonstrates potential strategies to further engineer HACS, ACR, ACT and ACK-PTA enzymes for improved activity and selectivity toward desired substrate(s). Approaches described in EXAMPLE 5 and 6 can be applied in other variants not just for 2^{nd} round HACS variants but also for other ACR, ACT and ACK-PTA variants. Structure modeled by AlphaFold followed by homology guided alignment will allow identification of active site residues as demonstrated in EXAMPLE 5. These key residues can then be targeted for directed evolution via saturation mutagenesis. Both simultaneous and iterative mutagenesis can be considered for the directed evolution. Alternatively, DNA shuffling of multiple variants with high expression, activity or substrate specificity can be shuffled for identifying candidates with higher catalytic efficiency. Random mutagenesis of candidate genes via error prone PCR is an option as well.

To increase throughput of screening method, selection-based screening method can be used. For screening of ACR on formaldehyde oxidation activity, we can leverage the toxicity of formaldehyde. E. coli with formaldehyde detoxification pathway (frmA) deleted cannot survive under submilimolar concentration of formaldehyde. Cells harboring ACR with high catalytic efficiency (k_{cat}/Kₘ) can rapidly convert formaldehyde to substantially less toxic formyl-CoA, allowing survival in the presence of other nutrients for cell maintenance and growth.

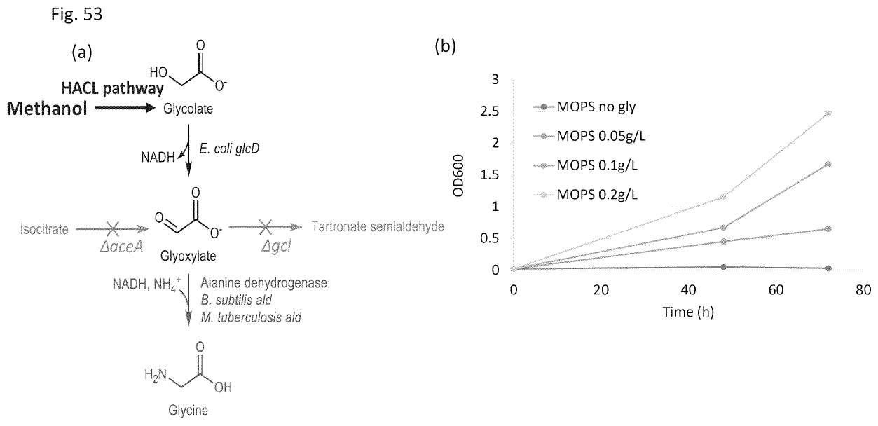

We have also developed a selection-based screening platform for glycolate production via glycine auxotroph strain. As a host for the selection platform, we engineered a glycine auxotroph strain of E. coli based on MG1655(DE3) with knockouts for glycine production and utilization (ΔaceA Δkbl ΔltaE ΔglyA), which forced the strain to grow only with the glycine supplementation (FIG. 53A). Glycolate can first be oxidized to glyoxylate catalyzed by E. coli glcD, followed by promiscuous activity of heterologous alanine dehydrogenases catalyzing glyoxylate reduction to glycine (FIG. 53A). Alanine dehydrogenases from Mycobacterium tuberculos and Bacillus subtilis are shown to have activity with glyoxylate to yield glycine (J. Bacteriol. 194:1045-1054, 2012; Biochemistry 20:5650-5655, 1981).

For gene deletions, CRISPR is used based on the method developed in Appl. Environ. Microbiol. 81:2506-2514, 2015). First, the host strain is transformed with plasmid pCas, the vector for expression of Cas9 and λ-red recombinase. The resulting strain is grown under 30° C. with L-arabinose for induction of λ-red recombinase expression, and when OD reaches ˜0.6, competent cells are prepared and transformed with pTargetF (AddGene 62226) expressing sgRNA and N20 spacer targeting the locus and template of insertion of target gene. The template is the deleted gene with ˜500 bp sequences homologous with upstream and downstream of the insertion locus, constructed through overlap PCR with usage of Phusion polymerase or synthesized by GenScript (Piscataway, NJ). The way to switch N20 spacer of pTargetF plasmid is inverse PCR with the modified N20 sequence hanging at the 5′ end of primers with usage of Phusion polymerase and followed by self-ligation with usage of T4 DNA ligase and T4 polynucleotide kinase (New England Biolabs, Ipswich, MA, USA). Transformants that grow under 30° C. on solid media (LB+Agar) supplemented with spectinomycin and kanamycin (or other suitable antibiotic) are isolated and screened for the chromosomal gene insert by PCR. The sequence of the gene insert, which is amplified from genomic DNA through PCR using Phusion polymerase, is further confirmed by DNA sequencing. The pTargetF can then be cured through IPTG induction, and pCas can be cured through growth under higher temperature like 37-42° C.

The resulting glycine auxotroph strain was transformed with a vector constitutively expressing alanine dehydrogenase from Mycobacterium tuberculosis (MtAld) or Bacillus subtilis (BsAld). When the strains are inoculated in minimal media (M9) with 5 g/L glucose, they failed to grow without glycine supplementation exhibiting glycine auxotrophy. Out of the two candidates, a strain harboring BsAld started to grow with glycolate instead of glycine supplementation (FIG. 28B) indicating glycolate is successfully being converted to glycine via native glcD and heterologously expressed BsAld genes. It also shows that only minimal supplementation of 50 mg/L glycolate was sufficient to see the growth, indicating suitable platform for selecting-based screening of HACS, ACR, ACT and ACK-PTA variants for glycolate production from C1 compounds.

Sequence information for certain of the examples and embodiments described herein are as follows:

>JGI15 HAK63664.1:
MAKSEGKVNGATLMARALQQQGVQYMFGIVGFPVIPIAIAAQREGITYIGMRNEQSASY
AAQAASYLTGRPQACLVVSGPGVVHALAGLANAQVNCWPMLLIGGASAIEQNGMGAF
QEERQVLLASPLCKYAHQVERPERIPYYVEQAVRSALFGRPGAAYLDMPDDVILGEVEE
AAVRPAATVGEPPRSLAPQENIEAALDALQSAKRPLVIVGKGMAWSRAENEVRQFIERT
RLPFLATPMGKGVMPDDHPLSVGGARSHALQEADLVFLLGARFNWILHFGLPPRYSKD
VRVIQLDLSAEEIGNNRQAEVALVGDGKAIVGQLNQALSSRQWFYPAETPWREAIAAKI
AGNQAAVAPMIADNTSPMNYYRVYRDIAARLPRNAIIVGEGANTMDIGRTQMPNFEPR
SRLDAGSYGTMGIGLGFAVAAAAVHPGRPVIAVQGDSAFGFSGMEFETAARYGMPIKVI
ILNNGGIGMGSPAPRDGQPGMPHALSHDARYERIAEAFGGAGFYVTDSAELGPALDAA
MAFKGPAIVNIKIAATADRKPQQFNWHG
>JGI19 OGA51379.1:
MAEINGAALIAKCLKQQGVKELFGVVGIPVTGIANAAQKEGIRYIGTRHEQAAGFAAQA
VSYLRGHVGVALTVSGPGMTNAITALGNAWANCWPMLLLGGSTDLAFAHRGGFQVAP
QMEAARPFCKWVAQPARVEDIPHLIEMGVRTAWYGRPGPVYIDLPADIIEAMVDEASLT
YPGPVSPPIRMAAPPELVAEAVQTLRSARKPLLIVGKGAAWSDAATEVRRIVDSTNIPVL
PTPMGKGVVPDDHPSIVSAARSYALKNADLIVLAGARLNWILHFGMPPRFNPETRVIQID
LAQEEIGNNLPATVGLTGDLKAILAQMVAQLEETPWKCDDRGAWKAGLAAEVAKKKT
ELKPALVSDEVPMGYFRPLQEIQKVLPRDAIIVSEGASTMDISRSVLENYQPRNRLDAGS
WGTMGGATGFALASAVVHPERRVIALMGDASFGFSGMEVEVAARHRLPITWIVFTNGG
IVSGVANLPKDGPLPVNVFQPGARYEKIMEAFGGKGFYCETPDQLARALRTAFDSGETA
LINVAIAPTAKKAPQTYSHWSSR
>JGI20 PWB41796.1:
MGQITGAQIVARALKQQGVEYMFGIVGIPVIPIAMFAQREGIKFYGFRNEQSASYAAAA
VGYLTGRPGVCLGVSGPGMIHGVAGMANAWSNCWPMILIGGANDSYQNGQGAFQEAP
QIEAARPFAKYCARPDSLARLPFYVEQAVRTSIYGRPGAVYLDLPGDIITGAMEEEDVHF
PPRCPDAPRMMAPQESIDAAMAALKSAERPLVIVGKGAAYSRAENEVREFLETTQLPYL
ASPMGKGVMPDDHPLSIAPARSAALLGADVILLMGARLNWMMHFGHPPRFDPKVRVIQ
MDISAEEIGTNVPTEVALVGDAKAITTQLNASLKQQPWQYPSETTWWTGLRKKIDENG
ATVAEMMADESVPMSYYRVYREIRDLIPNDAIIQNEGASTMDIGRTLMPNFLPRHRLDA
GSFGTMGVGLGQAIAAAAVHPDKHVFCIEGDSAFGFSGMEVETAARYGMKNITFIIINN
NGIGGGPDTLDPTRVPPSAYTPNAHYEKMAEIYGGKGYFVTEPSQLRPALEEAIKADKP
AIVNIMISATSQRKPQQFAWLTR
>JGI23 OWB57166.1:
MTTIDGSEVIAESLARLGVKTVFGIVGIPVVEVADALINKGIKFIGFRNEQAASYAASVYG
YLTQQPGVLLVVGGPGLVHALAGIYNSQSNKWPLLVLAGSSSSSEIYRGGFQELDQVSL
MTSTFAKFSAKPPSISRVPELITKAFRLSISGKPGPTYIDLPADIIQSKIDSTDGVKYLQSVIP
YTIEDIPKSVAPVNKLRQAVELIKSAQYPLLVVGKGASNCPRAVRNFVAEHMIPFLPTPM
GKGVVPDSSEFNVSSARSDALRHADVIILAGARLNWMLHHGDFPKFKKNVKFIQIDLDS
DEFGDNSNDSLKYGLYGDIGLTIESLNIALGKEHLVNSMLPVIETAKLKNIKKLELKGSV
TPEQSESLMNHNQALTIITDSLGLKYDDTVFVSEGANTMDISRVVIPINYPKQRLDAGTN
ATMGVGLGYAIAAKAASPEKLVIAIEGDSAFGFSAMEIETAIRSDLPLFIIVLNNSGIYRGV
SDVEKYAPFTNKPLPSTALSYKTRYDELGNSLGAVGMLVNNANELKLKMKECLDLYFN
ENKTIVLNVLIQSGAGTKLEFGWQNKPKSKL
>JGI24 KXN72624.1:
MSQEQLTGSSILAKSLKSLGVDVIFGIVGVPVVEVAEACIAEGIRFIGCRNEQSASFAAGA
WGYLNKRPGVCLTVSGPGVVNAISGLYNAQANCWPMILIGGSCETNQIGMGAFQELDQ
VDACRNYTKFSGKCADLETIPFIVNKAYQVSKAGRPGPTYVDLPADLIQATTSKLPKLPE
PFETPYCLPHTKDLSAAIEILKNSKRPLLVVGKGATYSRCENELKALVEEFNVPFLPTPM
AKGILPDNHSLNAGSARSLALRKADVIVLLGARLNWMMQFGNRLNPQTKIIHVDISPEE
FNINKKIDIGLFGNIPETIELIHQGLKKSGKSYSWIHFKNELQPNIEKNQEKLQKFLTAPLS
PLMNHQQALNTVEEVLSKQFNGDYFLVSEGARTMDVTRMLVSSHLPRRRLDAGTLGV
MGIGLGYALAGQLTHPDKKVVAIMGDSAFGFSAMEIETAARCKLPLIIIIINNNGIYHGLD
DIKSVPSDKLPSFTLMPETRYDLLANSVYGQGFLVKDSTQLQSALQKCFNFDGVSIVNV
MIDHRPASEGLYWLTREFSPAGQSKL
>JGIH65 PKN81274.1:
MPEGPVAEIDGQTIIARALKQQGVEAMFGVVGIPVTGIAAAAQREGIKYVGMRHEMPAT
YAAQAVSYLGGRLGTALAVSGPGVLNAVAAFANAWSNRWPMILIGGSYEQTGHLMGF
FQEADQLSALKPYAKYAERVERLERIPIYVAEAVKKALHGVPGPAYLELPGDIITAKIDE
SKVEWAPRVPDPKRTLSDPADVEAAIAALKTAQQPLIIVGKGVAASRAEVEIRAFVEKT
GIPYLAMPMAKGLIPDDHDQSAAAARSFVLQNADLIFLVGARLNWMLHFGLPPRFRPD
VRVVQLDFNPEEIGINVPTEVGMIGDAKATLSQLLDVLDRDGWRFPDDSEWVTAVSAE
ARQNAEAVQAMMQEDTQPLGYYRALRSIDERLPKDAIFVAEGASTMDISRTVINQYLPR
TRLDAGSFGSMGLGHGFAIGAATQFPGKRVICLQGDGAFGFAGTECEVAVRYNLPITWI
VFNNGGIGGHRAELFERDQKPVGGMSLGARYDILMQGLGGAAFNATNSDELDAAIEAA
LKIDGPSLINVPLDPDAKRKPQKFGWLTRTNE
>JGIH25 PFG74273.1:
MAELTGAQIVAKALKQQGVEYMFGVVGIPVVPIAVHAQREGIKFFGFRNEQAASYAAA
AIGYLTGRPGVCLAVSGPGMVHGIAGMANAWANCWPMILIGGANDSYQNGQGAFQEA
PQIETARPYAKYAARPDSTRRIPFFVEQAVRATIYGRPGAAYLDLPGDLITGSVDESEVHF
PPRCPDPPRTLAPWENIERALEALKSAERPLVIVGKGAAYARAEEEVRKFIDATQLPFLPT
PMGKGVVPDDHPLAISPARSFALQNADVVLLLGARLNWILHFGLPPRFDPKVRVIQVDI
AAEEIGNNVPAEVALVGDAKAIVGQMNEALTRAPWQYPAETTWWTGLRKKIEENAAT
VAEMMADESVPMGYYRVYRDIREYIPRDAIIVNEGANTMDIGRTLMPNFYPRHRLDAG
SFGTMGVGVGQAIAAAAVHPDKRVFCIEGDSAFGFSGMEVETAARYGLNNIVFIIINNN
GIGGGPDELDPTRVPPSAYTPNAHYEKMAEIYGGKGFFVTQPSELRPALEAALACDKPAI
VNIMISARSQRKPQQFAWLTR
>JGIH26 WP_158065972.1:
MAELTGAQIVAKALKQQGVEYMFGVVGIPVVPIAVHAQREGIKFFGFRNEQAASYAAA
AIGYLTGRPGVCLAVSGPGMVHGIAGMANAWANCWPMILIGGANDSYQNGQGAFQEA
PQIETARPYAKYAARPDSTRRIPFFVEQAVRATIYGRPGAAYLDLPGDLITGTVDESEVH
FPPRCPDPPRTLAPWENIERALDALKSAERPLVIVGKGAAYARAEEEVRTFIDMTQLPFL
PTPMGKGVVPDDHPLAISPARSFALQNADVVLLLGARLNWILHFGLPPRFDPKVRVIQV
DIAAEEIGNNVPAEVALVGDAKAIVEQMNEALSRAPWQYPAETTWWTGLRKKIEENAA
TVAEMMADESVPMGYYRVYRDIREYIPRDAIIVNEGASTMDIGRTLMPNFFPRHRLDAG
SFGTMGVGLGQAIAAAAVHPDKRVFCIEGDSAFGFSGMEVETAARYGLNNIVFIIINNNG
IGGGPDELDPTRVPPSAYTPNAHYEKMAEIYGGKGFFVTQPSELRPALEAALACDKPAIV
NIMISARSQRKPQQFAWLTR
>JGIH30 HEM18354.1:
MTTLDGATLIARSLRQQGVDYMFGIVGIPVVPVAIAFQREGGKFFGMRNEQAASYAAG
AVGYLTGRPGACLAVSGPGMVHAIAGLANAWANGWPMILLGGANDSYQNGQGAFQE
APQIEAARPFAKYCARPDSTRRIPFFIEQAVRYSIYGRPGPVYVDLPGDIITGTAEESEVRF
PPRCPDPPRALAPEENVRAALELLKQAERPLVIVGKGMAYARAEDEVREFIDRTRLPYLP
TPMGKGVIPDDHPFSVAPARSFALQNADVVFLMGARLNWILHFGLPPRFAPTVKTIQLDI
EPEEIGNNVPCTVPLVGDGKAIVGQLNAVLRGEPWEYPSETTWWTALRQKAAENEEMV
RQMEQDDSVPMGYYRVLREVRELLPKDAIVASEGANTMDISRTVIPNYFPRHRLDAGTF
GTMGVGLAQAIAAQVVHPDKKVVAIEGDSAFGFSGMEVEVMARYRLPITVIIVNNNGIS
GGPTQLDPNRVPPNAYLPNAHYEKIAEAFGGKGWFVTTPQELRPALEAALNSDTFSIVNI
MIDTRAGRKPQQFAWLTR
>JGIH41 MBI2761137.1:
MATINGATLLARSLKQQGVEYMFGIVGFPVQPIAGAAQREGITFIGMRNEQAASYAAHA
AGYLTGRPQACLVVSGPGVVHALAGLANAQSNCWPMILIGGASPTYQNGMGAFQEAP
QVKLAEPYCKYAHAVEQVDRIPYYVEQAVRSSIYGRPGATYLDMPDDIIRAEIEEEKVE
AKNTVPPPPRTQALDEDVESAVAALKSAERPLVIVGKGMAWSRAENEVREFIERSQLPF
LATPMGKGVMPDDHPLSVGAARSFVLQNADVVFLLGARLNWILHYGLPPRYSPNVRV
VQLDIAPEEIGANVPAEVGLVGDGKAVMRQVNRVLESSPWQYPSETTWRSGIANKIAEN
RVSTEAMMADDSSPMNYYHVLSTIRDMIPRDTIIASEGANTMDIGRTILNNYEPRTRLDA
GTFGTMGVGLGFAIAASVTNPTKRIIDVEGDSAFGFSGMEVETACRHKMPITFIIINNNGI
GGGPTEFDTSKPLPPNAYTPSAHYEKMMDAFGGKGYFVTESSELKPALEAALNTDGPSL
VNIMISNRATRKPQEFRWLTT
>JGIH61 MBT5774752.1:
MTDTTPATADTTNGAAAGETILGGVLLVRSLKQQGVDYMFGVVGFPVSELAGYAQDE
GIKYIGMRNEQAASYAAQAASYILGRPQACIVVSGPGVIHGLAGLANAKSNCWPMILIG
GASAVSQNGMGAFQEENQVEIARLVSKYAHSLDRVDRIPYYVEQAVRTSLYGRPGPAY
LDAPDDILTAEIPLSQIKTVPTVPDPPRPGVPERDIKAAVAALKSAERPLVIVGKGMAWS
RAENEVLEFIEKTQIPFLPTPMGKGVVDDDHPLAISPARTLALREADVVLLLGARLNWIL
HFGKPPRWAEDVRIIQVDIAAEEIGANVPAEVGLVGDGAAIVAQLNQALDEDGWQYPG
ETTWRSALKAKVDENVAVSAQLMADDSVPMNYYHPLQAIRDTLPEDTIIVSEGAGTMD
IGRTVLPNHGPRTRLDAGTYGTMGIGLGFAIGAAIAKPGTRIVDVEGDAAFGFSGMEYET
MVRHNLPITIVVINNNGIGGGVAELPEDRDPPPGVYLPSARYERIADMFGGRSYYVTQPE
ELEPALREANTGEGPAIVHIRIDPSAGRKPQQFGWHTPTN
>JGIR2 WP_185879480.1:
MDKDLLSVQQVRDLVKACKAAQKKYVEFSQEKMDKIVHEMSMEVRQYDEKLAKLAV
EETGFGKWEDKVIKNRFASTYIYDFIKNMKTVGILREENEVMEVGVPVGVIAGLIPSTNP
TSTTIYKILISLKAGNGIVISPHPNAKNCIIETANILKRAAIKAGAPEGLIGVIEIPTIQATDA
LMKHDDVSLILATGGEAMVRAAYSSGTPAIGVGPGNGPAFIERSANVKMAVKRIMQSK
TFDNGTICASEQSIIAEACNRTEIMKEVENQGGYFMPREDADKLARFILRPNGTMNPAIV
GKSAEVIANLAGIKIPLGTRVLLSEETTVSNSNPYSSEKLAPILAFYVEDNWEKACERSIEI
LNHEGRGHTMIIHSEDREVIREFALKKPVSRLLVNTPGSLGGIGATTNLAPALTLGCGAV
GGSSTSDNITPMNLINIRRVAWGVRELDYFRTENVEQTNVDSKDMEELIKKVLNEILNR
>JGIR5 WP_087641473.1:
MTTLDKDLASIQEVRNLLTEAKAAQESLAKMSQEQIDRICEAIAASAYEAREKLAKMAH
QETGFGIWQDKVVKNSFASKFVWDSIKEMKTVGILNEDKEQKVIDVAVPVGVVAGLIPS
TNPTSTVIYKALIAIKAGNAIVFSPHPNALQAILATVEIISKAAEKAGCPKGAIGCMLKPT
MQGTAELMKHQYTSLILATGGSAMVKAAYSSGTPAIGVGPGNGPAYIEKSADIPLAVKR
IMDSKTFDNGTICASEQSIIAETSNKAEVIAELKKQGAYFLSPEESAQLERYIMRPNGSMN
PQIVGKSVQAIAELTHLSVPKEAR VLIAEETKVGHKVPYSREKLAPILAFYTVGNWEEAC
ELAMDILYHEGAGHTMMIHSQNDEVIRQFGLKKPVSRVLVNTPGALGGIGATTNLAPAL
TLGCGAVGGSSTSDNISPANLFNVRRIAYGIRELEDLREQPVSSSGFNEEQLVDTLVERIL
AKLQ
>JGIR10 WP_202656015.1:
MTLLDKDLRSIQEARELIGKAKAAQSQLALLSQEQIDRIVKAIAEAGYDNREELAKIAAV
ETGFGKWEDKVLKNAFASQAVYESLKDLKTIGILKEDMQQKVMEIGVPLGVIAALIPST
NPTSTTIYKAMISLKAGNAIIFSPHPNAINCILETVRVIKEAAVKAGCPSDAISCMSIPAIEG
TETLMKHKDVSLILATGGSAMVKAAYSSGTPAIGVGPGNGPAFIERTANIPLAVKRIFDS
KTFDNGVICASEQSIVVEECIREEVIEECSKQGGYFLSERERKQLEKFIMRSNGTMNPAIV
GKSVEQIAKLAELNIPDGTRVLIAKESRVGRDVPYSREKLAPILAFYTEKDWQAACERCI
QLLLNEGAGHTLIIHSENEEVIKQFALKKPVSRLLVNTPGALGGIGATTNLVPALTLGCG
AVGGTSTSDNIGPLNLINIRRVAYGVKELEDLRENTPTCEPSFGVCDQKELIESIVKQVLA
QLH
>JGIR13 MBN6206692.1:
MMEMDKDLQSIHEARTLIGQAKEAQRQLAKLGQEDIDHIVKAMAEAAYEHRERLAKL
AVEDTGFGIVKDKVLKNLFASYGVYRAIKDMKTVGIINEDEQEKIVEVAVPVGVIAALV
PSTNPTSTVMNKALIAIKAGNAVVFSPHPSALNCILETTRILAEAAEAAGCPKGAITSMTK
PTMQGTDTLMKHRDVSLILATGGSAMVKAAYSSGTPAIGVGPGNGPAFIERSANVKQA
VKRIIDSKTFDNGVICASEQSVIVEADHKEVVVEEFKRQHAYFLSKEEAAKLEKFIMRPN
GTMNPQIVGKSALFLADLAGISVPSNTRVLIAEEDKVGKDVPFSREKLSPILAFYIEKDW
RAALDRSIEILLNEGAGHTMTVHSEKEEIIRAFTLEVPVFRLLVNTSATLGAIGATTNLLP
AYTLGCGALGNGSTSDNVGPMNLLNIKRVAIGIKDLAEIESESNNTKLSSAELNEDMVER
VVEQVLRQLYVMS
>JGIR14 WP_051217603.1:
MEMLDKDLRSIQEVRDLIKKAKEAQAKLAVMTQAQIDAIVKAIADAGYAHREKLAKM
ANEETGFGRWEDKIVKNAFASKHVYESIKDMKTVGIINDDKAHKVMDVAVPVGVVAG
LIPSTNPTSTVIYKALISLKAGNSIVFSPHPNALKSILETVKVINDAAVQAGCPEGAIASMT
VPTIQGTDQLMKHKDTSVILATGGEAMVKAAYSSGTPAIGVGPGNGPAFIEKSANFELA
VKRILDSKTFDNGTICASEQSVIVEACSKEAVMAEFKKQGAYFLTAEEAVQLGKFIMRA
NGTMNPQIVGRSVDHIAKLANLNVPAGTRVLIAEETSVGRNVPYSREKLAPILAFYTEDN
WEAACARSIEILNGEGAGHTMMIHSENEEIIRQFALKQPVSRLLVNTPGALGGIGATTAIA
PALTLGCGAVGGSSTSDNVSPMNLLNIRKLTYGLRELEDLVEQPTTQAAPAAATISQDD
KEQLISMIVARILEKM
>JGIRT45 2G39_1:
MYRDRVRLPSLLDKVMSAAEAADLIQDGMTVGMSGFTRAGEAKAVPQALAMRAKER
PLRISLMTGASLGNDLDKQLTEAGVLARRMPFQVDSTLRKAINAGEVMFIDQHLSETVE
QLRNHQLKLPDIAVIEAAAITEQGHIVPTTSVGNSASFAIFAKQVIVEINLAHSTNLEGLH
DIYIPTYRPTRTPIPLTRVDDRIGSTAIPIPPEKIVAIVINDQPDSPSTVLPPDGETQAIANHLI
DFFKREVDAGRMSNSLGPLQAGIGSIANAVMCGLIESPFENLTMYSEVLQDSTFDLIDAG
KLRFASGSSITLSPRRNADVFGNLERYKDKLVLRPQEISNHPEVVRRLGIIGINTALEFDIY
GNVNSTHVGGTKMMNGIGGSGDFARNAHLAIFVTKSIAKGGNISSVVPMVSHVDHTEH
DVDILVTEQGLADLRGLAPRERARVIIENCVHPSYQAPLLDYFEAACAKGGHTPHLLRE
ALAWHLNLEERGHMLAG
>JGIRT51 SJZ60628.1:
MSTSDVLNPEEVALVLREKVSPILRGHGGDLVLSHIRGKSIYIRFTGACRGCPAALETAE
RTVQAVLREHFGDEDIDAVLDNGVSEDLINQAKQILQKSKKIMNEILAQYKSKIVSADD
AVKVIKNGERVSLSHAAGVPQVCVDALVRNAEHFQGVEIYHMLCLGEGKYMLPEMAP
HFRHVTNFVGGNSRQAVAENRADFIPAFFYEVPTLFRKGILPIDVAIVQLSMPDAEGYCS
FGVSSDYTKPSTEVARVVIGEINAQTPYVHGDNKIHISKLDYIVLADYPLYTIPKAPIGPV
EEAIGRNCAELVEDGSTLQLGIGAIPDAALLFLKDKKDLGIHTEMFADGVIELVRAGVIT
GKKKSLHPGKMVATFLMGTEEVYKFAHNNPDVELYPVDYVNDPRTVAMNDNMVSINS
CIEVDLMGQVVSETIGPKQFSGTGGQVDYVRGATWSKNGKSIMAMPSTARKGAASRIV
PMIAEGASVTTLRNDVDYVVTEYGIARLKGRSLRQRAEALISIAHPDFREELMKVYRERF
E
JGIK1
>JGIK1-ACK AKJ38693.1:
MNWGLNMKVLVINAGSSSLKYQLIDMINESPLAVGLCERVGIDNSIITQKRFDGKKLEK
QVDLPTHRVALEEVVKALTDPEFGVITDMGEINAVGHRVVHGGEKFTTSALFDAGVEE
AIRDCFDLAPLHNPPNMMGITACAEIMPGTPMVIVEDTAFHQTMPAYAYMYALPYDLY
EKYGVRKYGFHGTSHKYVAGRAALMLGKPIEDTKIITCHLGNGSSIAAVKGGKSIDTSM
GFTPLEGVAMGTRCGSIDPAVVPFIMDKEGLSSREIDTLMNKKSGVLGVSGISNDFRDLD
EAASHGNERAELALEIFAYSVKRVIGEYLAVLNGADAIVFTAGIGENSASIRKRILAGLD
GLGIKIDEEKNKIRGQEIDISTPDSSVRVFVIPTNEELAIARETKEIVETEAKLRSSVPV
>JGIK-PTA AKJ38694.1:
MVTFLEKISERAKKLNKTIALPETDDIRTLQAAAKAIERGVANIVLIGDEAKIKELAGDLD
LSKAKIVNPETYEKKDEYIQAFYELRKHKGITLESAAEVMKDYVYFAVMAAKLDEVDG
VVSGAVHSSSDTLRPAVQIVKTAPGAALASAFFIIAVPDCEYGSDGTFLFADSGMVEMPS
VEDVANIAVISAKTFELLVQDDPYVAMLSYSTKGSAHSKLTEATIASTKLAQELAPDIPID
GELQVDAAIVPKVAASKAPGSPVAGKANVFIFPDLNAGNIAYKIAQRLAKAEAYGPITQ
GLAKPINDLSRGCSDEDIVGAIAITCVQAAAQDK
JGIK8
>JGIK8-ACK HIX51076.1:
MKILVVNAGSSSLKYQLFDMDTESVIVKGGVERIGIRGSVLHHKWAQGEKVIEQDMPN
HKVAMQAVLDALVHPEYGAIHSMSEIDAVGHRVLHSGGDFDGSVLLDDEVLKICKKNA
ELGPLHMPANILGIEACREVMPHTPMALVFDTAFHATMPPHAYMYAVDYDDYKNYKV
RKYGFHGTSHKYVSQEAIKYLGRGAAGTKIITAHLGGGSSLSAVMDGKCVDTSMGFTP
LAGVPMGTRSGDIDPAVLEFLAAKKGYTVLDCINYLNKQCGVAGISGISSDFRDLTKAA
AEGNERAQLALDMFAYAVKKYIGSYIAAMDGLDCLVFTAGIGENTWQVREMICDKMD
CFGIALDAEKNRLKNDGAIHDITGEGSKVKVLVIPTNEELVIARETKELVEA
>JGIK8-PTA HIX51075.1:
MADFFNKVKDKMSAVKDKLGEMIEKEEDTFLYRIKKRASELNKRIVLCEGEDSRVVKA
ASVAAKQGVAKIVLLGNAEQIAKDNPDIDLSAVEIVDPAASEKRAEYAALLYQLRQAKG
MTQEEAEKLSYDNTYFGVLMVKAGDADGLVSGACHSTANTLRPGLQIVKAAPGVPLVS
SCFFMVAPPAGNQYCEDGVFIYSDCGLNENPNSEQLAEIAIISAKTAEKIAGLEPRVAML
SFSTKGSAKHADIDKVTAAYRIAKEKAPDLALDGELQLDSAIVPAVAKSKAPGSKVAGH
ANVLIFPDLDAGNIGYKLTERLGGFMAVGPVCQGFAKPINDLSRGCKWEDIVATIAITAL
QTQM
JGIK31
>JGIK31-ACK WP_022744670.1:
MKILVINCGSSSLKYQLINMEDKGVLAQGLVERIGISGSILTQKVDGRDKYVIESPLKDH
QEAIDLVLRTLVDDNQGVIKSMEEISAVGHRVVHGGEKYATSVVVTEEVIKNLEDFIKL
APLHNPPNIIGIRACQALMPNTPMVAVFDTAFHQTMPEKAFMYPLPYELYKEDHIRRYG
FHGTSHKYVAGEVAKWMKKDIKDIKTITCHLGNGVSVTAVNGGQSIDTTMGFTPLDGII
MGSRSGSIDPAIVTYLVKEKGYSIDEVNEILNKKSGVLGISGLGTDFRDIRAAVEERNDK
RALLTMDIYGYQIKKQIGAYAAAMAGVDAIVFTAGIGEHAPEIRVRALTDMEFLGIELD
VDKNDNQNIGDGMEISKPSSKVKVFVIPTNEELMIAEETLELIQK
>JGIK31-PTA WP_022744669.1:
MNLMQKIWDAAKSDKKKIVLPEGNEERTIVAAEKINRLGLAHPILIGNKEEIINKGHALD
VDLSQVEIIDPAESENLEKYITAFYELRKNKGITLEKAEKIVKDPLYFATMMVKLDDADG
MVSGAVHTTGDLLRPGLQIIKTAPGVSVVSSFFIMEVPNSSYGEDGLLLFADCAVNPMP
NEDQLAAIAIATAETAKRLCNMDPKVAMLSFSTKGSADHEVVDKVRNATKKANELRPD
LDIDGELQLDASIVEKVANQKAPGSKVAGKANVLVFPDLQAGNIGYKLVQRFANAKAI
GPVCQGFAKPINDLSRGCSSDDIIDVVALTAVQAQNIK

1. A recombinant microorganism expressing a 2-hydroxyacyl-CoA synthase, wherein the 2-hydroxacyl-CoA synthase is enzymatically capable of least 2-fold, alternatively 3-fold greater rate of formation of a 2-hydroxyacyl-CoA from a carbonyl-containing compound and formyl-CoA compared to the Rhodospiralles bacterium URHD0017 2-hydroxyacyl-CoA synthase.

2. The recombinant microorganism of claim 1, wherein the carbonyl-containing compound is selected from the group consisting of an aldehyde and a ketone.

3. (canceled)

4. (canceled)

5. (canceled)

6. The recombinant microorganism of claim 1, further comprising an enzyme catalyst that converts a substrate to the carbonyl-containing compound.

7. The recombinant microorganism of claim 1, further comprising an enzyme catalyst that converts the 2-hydroxyacyl-CoA to an organic chemical product.

8. (canceled)

9. (canceled)

10. (canceled)

11. (canceled)

12. (canceled)

13. (canceled)

14. (canceled)

15. (canceled)

16. (canceled)

17. (canceled)

18. The recombinant microorganism of claim 39, wherein the one carbon substrate is formaldehyde and the enzyme catalyst that produces formyl-CoA is:
a. an acyl-CoA reductase (acylating aldehyde dehydrogenase) that catalyzes the conversion of formaldehyde to formyl-CoA; or
wherein the one carbon substrate is methanol and the enzyme catalysts that produce formyl-CoA are:
a. a methanol dehydrogenase catalyzing the conversion of methanol to formaldehyde; and
b. an acyl-CoA reductase (acylating aldehyde dehydrogenase) catalyzing the conversion of formaldehyde to formyl-CoA; or
wherein the one carbon substrate is methane and the enzyme catalysts that produce formyl-CoA are:
a. a methane monooxygenase catalyzing the conversion of methane to methanol;
b. a methanol dehydrogenase catalyzing the conversion of methanol to formaldehyde; and
c. an acyl-CoA reductase (acylating aldehyde dehydrogenase) catalyzing the conversion of formaldehyde to formyl-CoA; or
wherein the one carbon substrate is formate and the enzyme catalysts that produce formyl-CoA are:
a. an acyl-CoA synthase catalyzing the conversion of formate to formyl-CoA; or
b. a formate kinase catalyzing the conversion of formate to formyl-phosphate and a phosphate formyl-transferase catalyzing the conversion of formyl-phosphate to formyl-CoA; or
wherein the one carbon substrate is carbon dioxide and the enzyme catalysts that produce formyl-CoA are:
a. a carbon dioxide reductase catalyzing the conversion of carbon dioxide to formate; and
b. an acyl-CoA synthase catalyzing the conversion of formate to formyl-CoA; or
c. a formate kinase catalyzing the conversion of formate to formyl-phosphate and a phosphate formyl-transferase catalyzing the conversion of formyl-phosphate to formyl-CoA.

19. (canceled)

20. (canceled)

21. (canceled)

22. (canceled)

23. The recombinant microorganism of claim 7, wherein the product is an aldehyde and wherein the enzyme catalysts converting the 2-hydroxyacyl-CoA to said product is:
a. an acyl-CoA reductase catalyzing the conversion of the 2-hydroxyacyl-CoA to the aldehyde; or
wherein the product is an alcohol and wherein the enzyme catalysts converting the 2-hydroxyacyl-CoA to said product are:
a. an acyl-CoA reductase catalyzing the conversion of the 2-hydroxyacyl-CoA to the aldehyde; and
b. an alcohol dehydrogenase (aldehyde reductase) catalyzing the conversion of the aldehyde to the alcohol; or,
wherein the product is a carboxylic acid and wherein the enzyme catalysts converting 2-hydroxyacyl-CoA to said product is:
a. a thioesterase catalyzing the conversion of the 2-hydroxyacyl-CoA to the carboxylic acid.

24. (canceled)

25. (canceled)

26. (canceled)

27. (canceled)

28. (canceled)

29. (canceled)

30. (canceled)

31. (canceled)

32. The recombinant microorganism of claim 1, wherein the microorganism is a bacteria.

33. The recombinant microorganism of claim 32, wherein the bacteria is E. coli.

34. (canceled)

35. The recombinant microorganism of claim 1, wherein the 2-hydroxyacyl-CoA synthase has at least 90% or greater identity to SEQ ID NO: 1 (JGI15) or to SEQ ID NO: 3 (JGI20).

36. The recombinant microorganism of claim 35, wherein the 2-hydroxyacyl-CoA synthase has the sequence of SEQ ID NO: 1.

37. The recombinant microorganism of claim 35, wherein the 2-hydroxyacyl-CoA synthase has the sequence of SEQ ID NO: 3.

38. The recombinant microorganism of claim 35, wherein the 2-hydroxyacyl-CoA synthase comprises one or more mutations relative to SEQ ID NO: 3, optionally wherein the mutations are N461del and R480ins relative to SEQ ID NO: 3, A253G and P254G relative to SEQ ID NO: 3, and/or at positions L549H, T550G, and R551del relative to SEQ ID NO: 3.

39. The recombinant microorganism of claim 1, wherein the microorganism further expresses an enzyme catalyst that produces the formyl-CoA from a one carbon substrate.

40. A method for the formation of a 2-hydroxyacyl-CoA from a carbonyl-containing compound and a formyl-CoA, wherein the formation of the 2-hydroxyacyl-CoA is catalyzed by a 2-hydroxyacyl-CoA synthase, wherein the 2-hydroxyacyl-CoA synthase is enzymatically capable of least 2-fold, alternatively 3-fold greater, rate of formation of a 2-hydroxyacyl-CoA from a carbonyl-containing compound and formyl-CoA compared to the Rhodospiralles bacterium URHD0017 2-hydroxyacyl-CoA synthase.

41. The method of claim 40, wherein the 2-hydroxyacyl-CoA synthase has at least 90% or greater identity to SEQ ID NO: 1 (JGI15) or to SEQ ID NO: 3 (JGI20).

42. The method of claim 40, further comprising the formation of the formyl-CoA from a one carbon substrate, wherein the formation of the formyl-CoA is catalyzed by an enzyme catalyst.

43. The method of claim 40, further comprising:
i) the conversion of a substrate to the carbonyl-containing compound, wherein the conversion to the carbonyl-containing compound is catalyzed by an enzyme catalyst; or
ii) the conversion of the 2-hydroxyacyl-CoA to an organic chemical product, wherein the conversion of the 2-hydroxyacyl-CoA to the organic chemical product is catalyzed by an enzyme catalyst.

44. The method of claim 42, wherein:
i) the one carbon substrate is formaldehyde and the enzyme catalyst that catalyzes the formation of the formyl-CoA is:
a. an acyl-CoA reductase (acylating aldehyde dehydrogenase) that catalyzes the conversion of the formaldehyde to the formyl-CoA;
ii) the one carbon substrate is methanol and the enzyme catalysts that catalyze the formation of the formyl-CoA are:
a. a methanol dehydrogenase catalyzing the conversion of the methanol to formaldehyde; and
b. an acyl-CoA reductase (acylating aldehyde dehydrogenase) catalyzing the conversion of the formaldehyde to formyl-CoA;
iii) the one carbon substrate is methane and the enzyme catalysts that catalyze the formation of the formyl-CoA are:
a. methane monooxygenase catalyzing the conversion of the methane to methanol;
b. a methanol dehydrogenase catalyzing the conversion of the methanol to formaldehyde; and
c. an acyl-CoA reductase (acylating aldehyde dehydrogenase) catalyzing the conversion of the formaldehyde to the formyl-CoA;
iv) the one carbon substrate is formate and the enzyme catalysts that catalyze the formation of the formyl-CoA are:
a. An acyl-CoA synthase catalyzing the conversion of the formate to the formyl-CoA; or
b. A formate kinase catalyzing the conversion of the formate to formyl-phosphate and a phosphate formyl-transferase catalyzing the conversion of the formyl-phosphate to the formyl-CoA; or
v) the one carbon substrate is carbon dioxide and the enzyme catalysts that catalyze the formation of the formyl-CoA are:
a. a carbon dioxide reductase catalyzing the conversion of the carbon dioxide to formate; and
b. an acyl-CoA synthase catalyzing the conversion of the formate to the formyl-CoA; or
c. a formate kinase catalyzing the conversion of the formate to formyl-phosphate and a phosphate formyl-transferase catalyzing the conversion of the formyl-phosphate to the formyl-CoA.

45. The method of claim 40, wherein the carbonyl-containing compound is selected from the group consisting of an aldehyde and a ketone.

46. The method of claim 45, wherein the aldehyde has at least one substituent group wherein the substituent group is a hydroxyl, a carbonyl, a carboxyl, an alkyl, an alkenyl, an alkynyl, an amine.

47. The method of claim 40, wherein the enzymes are contained in a recombinant microorganism harboring genes for expressing each enzyme and optionally, wherein the substrates are contacted with the recombinant microorganisms containing the enzyme catalysts in an aqueous media optionally containing buffers, salts, vitamins, or minerals.

48. A recombinant 2-hydroxyacyl-CoA synthase, wherein the 2-hydroxyacyl-CoA synthase comprises one or more mutations relative to SEQ ID NO: 3 (JGI20), wherein 2-hydroxyacyl-CoA synthase is enzymatically capable of least 2-fold, alternatively 3-fold greater rate of formation of a 2-hydroxyacyl-CoA from a carbonyl containing compound and formyl-CoA compared to the Rhodospirillales bacterium URHD00172-hydroxyacyl-CoA synthase.

49. The recombinant 2-hydroxyacyl-CoA synthase of claim 48, wherein the mutations are N461del and R480ins relative to SEQ ID NO: 3, A253G and P254G relative to SEQ ID NO: 3 and/or at positions L549H, T550G, and R551del relative to SEQ ID NO: 3.
